(19) United States
(12) Patent Application Publication
(10) Pub. No.: US 2012/0202202 A1
(43) Pub. Date: Aug. 9, 2012

(54) METHODS FOR DETECTING RARE CIRCULATING CANCER CELLS USING DNA METHYLATION BIOMARKERS

(76) Inventors: Michael Xia WANG, Columbia, MO (US); Charles W. Caldwell, Columbia, MO (US); Kristen H. Taylor, Columbia, MO (US); Srilatha Nalluri, Augusta, GA (US); Dali Zheng, St. Louis, MO (US)

(21) Appl. No.: 13/360,649

(22) Filed: Jan. 27, 2012

Related U.S. Application Data
(60) Provisional application No. 61/462,127, filed on Jan. 28, 2011.

(51) Int. Cl. C12Q 1/68

(52) U.S. Cl. 435/6.11

(57) ABSTRACT

Provided are new and improved methods for detecting circulating tumor cells and tumor cell DNA in patient blood or other biofluid samples. Particular aspects comprise three steps: DNA extraction from patient samples, DNA digestion with multiple selected methylation-sensitive enzymes, and target amplification by a conventional or a real-time PCR with specific probe and/or primers. Also provided are a total of 40 tumor-specific DNA methylation loci as biomarkers having substantial utility and specificity in major types of human malignancies including hematopoietic and solid tumors.

### CROSS-REFERENCE TO RELATED APPLICATIONS

This application claims the benefit of priority to U.S. Provisional Patent Application No. 61/462,127, filed 28 Jan. 2011 and entitled “DNA METHYLATION BIOMARKERS FOR RARE CIRCULATING CANCER CELL DETECTION,” which is incorporated herein by reference in its entirety.

### FIELD OF INVENTION

The present invention relates to a sensitive quantitative real-time PCR method using specific DNA hypermethylation as biomarker for cancer detection, more specifically, for early detection, diagnosis, and monitoring the circulating tumor cells and tumor cell DNA in a patient blood sample.

### SEQUENCE LISTING

A Sequence Listing, comprising 139 SEQ ID NOS, is submitted herewith in both .txt and .pdf formats, is part of the present application, and is incorporated herein by reference in its entirety.

### BACKGROUND OF INVENTION

Approximately 90% of cancer deaths are caused by the hematogenous spread and subsequent growth of tumors at distant organs; this process is termed “metastasis.” Emerging evidence indicates that the disseminating tumor cells present in the peripheral blood and bone marrow represent an early, rather than a late event in cancer development. These circulating tumor cells (CTCs) like “malignant seeds” are relevant to overt metastases and death [1, 2]. Clinically, the major obstacle to the cure of cancer is metastasis. If the tumors are detected before metastasis, the cure rate is near to 100%. Once metastasized, the tumor is usually incurable. Therefore, early detection and diagnosis of cancer before an overt metastasis has become a central issue for cure of cancer. On the other hand, most hematopoietic tumors are derived from bone marrow or lymphoid tissues and the leukemia and lymphoma cells naturally circulate in blood [3]. Early detection of CTC and leukemic and lymphoma cells and characterization of molecular signature of these tumor cells provide vital insight information for early diagnosis, early medical intervention, and thus save lives. An important molecular signature in cancer cells is aberrant DNA hypermethylation in functional genes. This epigenetic alteration is not only an early event in tumorigenesis, but a useful biomarker for cancer detection [4, 5].

Furthermore, during tumor progression, a small fraction of tumor cells constantly die by necrosis and/or apoptosis. Tumor cell DNA is released into blood or biofluids after lysis. These DNAs not only carry tumor genetic information (mutations), but also epigenetic alterations (DNA methylation). Aberrant DNA hypermethylation is the most common, often tumor-specific and detectable markers [6]. However, the levels of cell-free tumor DNA in blood are usually very low and the detection requires extremely sensitive and specific methods.

While morphology assessment was the golden-standard for the diagnosis of cancer, an integrated system of clinical features, imaging, endoscopy, biopsy, morphology, immunophenotype, genetic analysis has become the new standard of care in modern diagnostics of cancer. In recent years, additional cancer biomarkers such as proteins, DNA, mRNA, microRNA, either in a specific or a profiling assay, play important role in clinical diagnosis and patient management. This is especially important in early diagnosis, monitoring disease course and detecting minimal residual disease.

In the case of diagnosis of a hematopoietic malignancy, delineating cell lineage using various modalities is a starting point to categorize, classify and define a hematologic tumor [3]. Immunophenotyping by either flow cytometry or immunohistochemistry is used in routine diagnosis in the vast majority of hematopoietic malignancies [7].

Genetic abnormalities such as point mutations, copy number, amplification, expression levels, and chromosomal translocations detected by either molecular analysis or molecular cytogenetics [such as fluorescent in situ hybridization (FISH)] are increasingly utilized to define hematopoietic and other cancer cells [3, 7-9]. However, genetic analysis may not be a perfect method to detect malignancy. For instance, the chromosomal translocation t(14;18)(q32;q21), a hallmark for follicular lymphoma (FL), was detected in 75% of FL cases [10]. However, this translocation could be detected in up to 66% of healthy adults' peripheral blood with no evidence of FL when using a sensitive real-time PCR method [11]. Most importantly, not all cancers carry the uniform mutations. In fact, specific genetic mutations are detectable only in a small fraction of cancer patients that makes genetic detection difficulty and impractical [12].

Therefore, there is a need to provide a new and improved method/system for cancer detection.

### SUMMARY OF INVENTION

In one aspect of the invention, a new and improved method for detecting cancer cells and monitoring circulating tumor cells (CTCs) and tumor cell DNA in a patient's blood (or other biofluids) sample is described. The method utilizes specific cancer DNA methylation as biomarker combined with a sensitive and quantitative real-time PCR detection. The inventive method comprises three steps: DNA extraction from patient specimens, DNA digestion with multiple selected methylation sensitive enzymes, and a TaqMan probe or SYBR Green florescence-based real-time PCR amplification with specific probe and/or primers. The patient samples may be whole blood, buffy coat, isolated mononuclear cells, plasma or serum, and other biofluids.

In another aspect of the invention, a total of 40 DNA methylation biomarkers identified by the present method are described. These markers are typically located in the CG rich promoter or the first exon region (CpG island or CGI) of a gene. These genes include HOXD10, COX2, KLF4, SLC26A4, DLC-1, PCDHGA12A, RPIB9, SOX2, CXCR4, HIN1, SFRP2, DAPK1, CD44, CDH1, PGRB, OLIG2, NOR1, SOCS1, RECK, MAFB, p15, HOXD11, HOXA11, HOXA6, HOXA7, HOXD9, HOXA9, HOXC4, PCDHA13, HIC1, CDH13, HOXA4, PCDHA6, PCDHB15, PTPN6, APC, GSTP1, ADAM12, p16, and GABRBA. The newly described DNA methylation loci may be employed as biomarkers to detect major types of human malignancies including hematopoietic tumors, solid tumors, and cutaneous tumor.

Particular aspects provide methods for the diagnosis, prognosis or detection of circulating cancer cells in a subject, comprising: contacting genomic DNA, obtained from a biological sample of a human subject and having at least one genomic DNA target sequence selected from the CpG island group consisting of HOXD10, COX2, KLF4, SLC26A4, DLC-1, PCDHGA12A, RPIB9, SOX2, CXCR4, HIN1, SFRP2, DAPK1, CD44, CDH1, PGRB, OLIG2, NOR1, SOCS1, RECK, MAFB, p15, HOXD11, HOXA11, HOXA6, HOXA7, HOXD9, HOXA9, HOXC4, PCDHA13, HIC1, CDH13, HOXA4, PCDHA6, PCDHB15, PTPN6, APC, GSTP1, ADAM12, p16, GABRBA, and portions thereof, with a plurality of different methylation-sensitive restriction enzymes each having at least one CpG methylation-sensitive cleavage site within the at least one genomic DNA target sequence, wherein the at least one target sequence is either cleaved or not cleaved by each of said plurality of different methylation-sensitive restriction enzymes; amplifying the contacted genomic DNA with at least one primer set defining at least one amplicon comprising the at least one target sequence, or the portion thereof, having the at least one CpG methylation-sensitive cleavage site for each of the plurality of different methylation-sensitive restriction enzymes to provide an amplificate; and determining, based on a presence or absence of, or on a pattern or property of the amplificate relative to that of a normal control, a methylation state of at least one CpG dinucleotide sequence of the at least one target nucleic acid sequence, wherein a method for the diagnosis, prognosis or detection of circulating cancer cells in the human subject is afforded.

In certain embodiment, amplification comprises at least one of standard, multiplex, nested and real-time formats.

In particular embodiments, the at least one target sequence comprises the RPIB9 gene CpG island, or a portion thereof. In certain aspects, the at least one target sequence additionally comprises at least one of the PCDHGA 12 gene CpG island, and portions thereof. In certain aspects, the at least one target sequence additionally comprises at least one of the DLC-1 gene CpG island, and portions thereof. Particular aspects comprise amplification of a plurality of target sequences within the DLC-1 gene CpG island. In certain embodiments, the at least one target sequence additionally comprises (e.g., in addition to RPIB9) the PCDHGA 12 and DLC-1 CpG islands, or portions thereof.

In certain aspects, said methylation sensitive enzyme comprises at least two selected from the group consisting of Acil, HpaII, HinP1I, BstUI, Hha I, and Tai I. Particular embodiments comprise digestion with Acil, HpaII, HinP1I, and BstUI.

In certain aspects, the at least one genomic DNA target sequence comprises at least 3, at least 4, at least 5, or at least 6 methylation-sensitive restriction sites.

In particular embodiments, the at least one genomic DNA target sequence comprises at least four different methylation-sensitive restriction sites, and contacting comprises contacting the at least one genomic DNA target sequence with a respective four different methylation-sensitive restriction enzymes.

In certain embodiments, the biological sample comprises at least one of whole blood, buffy coat, isolated mononuclear cells, isolated blood cells, plasma, serum, bone marrow, and other body fluids (e.g., stool, colonic effluent, urine, saliva, etc.).

In certain aspects, the cancer comprises at least one of hematopoietic tumors, solid tumors, and cutaneous tumors, acute lymphoblastic leukemia (ALL), minimal residual disease (MRD) in acute lymphoblastic leukemia (ALL), acute myeloid leukemia (AML), lung cancer, breast cancer, ovarian cancer, prostate cancer, colon cancer, and melanoma.

Particular aspects comprise diagnosis or detection of at least one of acute lymphoblastic leukemia (ALL), minimal residual disease (MRD) in acute lymphoblastic leukemia (ALL), and acute myeloid leukemia (AML) in biofluids or tissue samples of either hematopoietic or solid tumors.

Particular aspects comprise diagnosis or detection of at least one of lung cancer, breast cancer, ovarian cancer, prostate cancer, colon cancer, and melanoma in biofluids or tissue samples comprising cancer cells.

In certain embodiments, the relative sensitivity in detecting cancer is one malignant cell or allele in one million normal cells or alleles (10⁻⁶).

In certain aspects, the relative sensitivity in detecting at least one of acute lymphoblastic leukemia (ALL), minimal residual disease (MRD), and acute myeloid leukemia (AML) is one malignant cell or allele in one million normal cells or alleles (10⁻⁶).

In certain aspects, the relative sensitivity in detecting at least one of lung cancer, breast cancer, ovarian cancer, prostate cancer, colon cancer, and melanoma is one malignant cell or allele in one million normal cells or alleles (10⁻⁶).

In particular embodiments, the biological sample is from a post-chemotherapy subject.

In particular embodiments, the cancer comprises acute lymphoblastic leukemia, and the at least on marker is selected from the group consisting of DLC-1, PCDHGA12A, CDH1, HOXD10, RPIB9, CD44, COX2, SOX2, KLF4, SLC26A, RECK, HOXA9, HOXD11, HOXA6, ADAM12, and HOXC4.

In particular embodiments, the cancer comprises chronic lymphocytic leukemia, and the at least on marker is selected from the group consisting of DLC-1, PCDHGA12A, HOXD10, CD44, COX2, HOXA9, HOXA4, HOXD11, and HOXA6.

In particular embodiments, the cancer comprises follicular lymphoma, and the at least on marker is selected from the group consisting of DLC-1, PCDHGA12A, CDH1, HOXD10, RPIB9, COX2, KLF4, HOXA9, HOXA6, HOXC4, and SLC26A4.

In particular embodiments, the cancer comprises mantle cell lymphoma, and the at least on marker is selected from the group consisting of DLC-1, PCDHGA12A, HOXD10, HOXA9, HOXD11, and HOXA6.

In particular embodiments, the cancer comprises Burkett lymphoma, and the at least on marker is selected from the group consisting of DLC-1, PCDHGA12A, CDH1, HOXD10, RPIB9, CD44, COX2, KLF4, HOXA9, HOXD11, HOXA6, HOXC4, and SLC26A4.

In particular embodiments, the cancer comprises diffuse large B-cell lymphoma, and the at least on marker is selected from the group consisting of DLC-1, PCDHGA12A, CDH1, HOXD10, RPIB9, COX2, KLF4, HOXA6, and SLC26A4.

In particular embodiments, the cancer comprises multiple myeloma, and the at least on marker is selected from the group consisting of DLC-1, PCDHGA12A, CDH1, COX2, KLF4, HOXA9, HOXD11, HOXA6, HOXC4, HOXD10, and SLC26A.

In particular embodiments, the cancer comprises acute myeloid leukemia, and the at least on marker is selected from the group consisting of PCDHGA12A, CDH1, HOXD10, CD44, CXCR1, KLF4, SLC26A, CDH13, HOXA9, HOXD11, HOXA6, HOXC4, ADAM12, and SLC26A4.

In particular embodiments, the cancer comprises myelodysplastic syndrome, and the at least on marker is selected from the group consisting of PCDHGA12A, SOCS-1, and HIN1.

In particular embodiments, the cancer comprises breast cancer, and the at least on marker is selected from the group consisting of DLC-1, PCDHGA12A, HOXD10, RPIB9, COX2, RECK, HOXA11, HOXA7, HOXA9, HOXD9, HOXD11, PCDHB15, PCDHA6, PCDHA13, PTPN6, HIC1, CDH13, GSTP1, ADAM12, p16, GABRBA, and APC.

In particular embodiments, the cancer comprises lung cancer, and the at least on marker is selected from the group consisting of PCDHGA12A, HOXD10, HOXA7, HOXA6, HOXA9, PCDHB15, PCDHA6, PCDHA13, PTPN6, GSTP1, and HIC1.

In particular embodiments, the cancer comprises colon cancer, and the at least on marker is selected from the group consisting of DLC-1, PCDHGA12A, HOXD10, RPIB9, CD44, COX2, SOX2, CXCR1, SLC26A, RECK, HOXA7, HOXA6, HOXA9, PCDHB15, PCDHA6, PCDHA13, PTPN6, ADAM12, p16, and HIC1.

In particular embodiments, the cancer comprises ovarian cancer, and the at least on marker is selected from the group consisting of PCDHGA12A, HOXD10, SLC26A, CDH13, and RECK.

In particular embodiments, the cancer comprises prostate cancer, and the at least on marker is selected from the group consisting of PCDHGA12A, HOXD10, COX2, HOXA7, HOXA6, HOXA9, HOXD11, HOXD9, PCDHB15, PCDHA6, PTPN6, HIC1, APC, CDH13, CDH5, HOXA11, GSTP1, p16, GABRBA, and HOXA7.

In particular embodiments, the cancer comprises melanoma, and the at least on marker is selected from the group consisting of PCDHGA12A, HOXD10, KLF4, and COX2.

### DESCRIPTION OF DRAWINGS

FIG. 1 is a schematic illustration of the inventive multiple methylation sensitive enzyme restriction PCR (MSR-PCR) method including a quantitative real-time platform (qMSR-PCR).

FIG. 2 illustrates the development of a conventional gel-based MSR-PCR method using DLC-1 gene in leukemia cell lines. (A) Different DNA methylome (genome-wide methylation pattern) between normal blood and leukemic cells. Genomic DNA from normal (lanes 1-4) and ALL cell lines (lanes 5-9) give rise to different methylation patters when digested with 4 methylation sensitive enzymes with AciI, HpaII, HinP1I, and BstUI except lanes 1 and 3, in which no enzymes were added. Lane 1-2: normal male; Lanes 3-4: normal female; Lanes 5-8: four ALL cell lines (lane 5, NALM-6; lane 6, MN-60; lane 7, SD-1; and lane 8, Jurkat). 100 ng of digested DNA was separated by electrophoresis at 120 V for 60 min in 1% agarose gel and visualized with the florescent dye SYBR Green 1. The 100 bp (lane M1) and 1 kb (lane M2) DNA ladders were included. (B) DLC-1 CpG island and the restriction map of PCR target regions. The island consists of an 824 bp at chromosome 8p21.3-22 (chr 8:13034462-13035285). Central regions A (160 bp) and B (238 bp) (black bar below the CpG island, restriction sites are indicated with arrows on the expanded line) with dense CG dinucleotides and multiple restriction sites were selected for PCR amplification. (C) Efficiency of DNA digestion by methylation sensitive enzymes. 250 ng of normal DNA from human blood (lanes 3, 5, 7, 9, 11) and B-ALL cell line NALM-6 (lanes 4, 6, 8, 10, 12) were digested with either a single enzyme or a combination (labeled above the lines). Lanes 1 and 2 are controls from normal male and female DNA digestion with no enzymes. W-PCR water control, M-100 bp DNA ladder. (D) Analytic sensitivity of MSR-PCR. Upper panel shows absolute sensitivity. After digestion with 4 enzymes, 80 ng of DNA from NALM-6 cell line was diluted in a 5× series starting from lane 4 and the targets of DLC-1A and β-actin-A were amplified with MSR-PCR. Lanes 1-2 were normal DNA without and with enzymes, respectively; Lane 3-water control. Middle panel shows relative sensitivity. A 10× serial dilution of DNA from NALM-6 was mixed with normal DNA from human blood to make a total of 250 ng DNA (lanes 7-11). Lanes 1-4 were DNA from normal male (lanes 1-2) and female (lanes 3-4) without enzymes (lanes 1 and 3) and with enzymes (lanes 2 and 4), respectively. Lane 5 contained 250 ng of normal DNA only. Lane 6 contained 250 ng of NALM-6 DNA only. The lower panel shows results from nested PCR. After amplification of a 10× dilution series of NALM-6 DNA with FF and BR primer pair in the 1^{st} PCR, aliquots of PCR products (383 bp) were re-amplified with an internal AF and AR primer pair in the 2^{nd} PCR. Lanes 1-5, W and M were as same as described in middle panel. All experiments in FIG. 2 were performed at least three times with the same results; a representative gel image is shown.

FIG. 3 is the validation of MSR-PCR method using 3 DNA methylation biomarkers in B-cell tumor cell lines and B-ALL patient samples. (A) Cell lines. Genomic DNAs from normal blood (lane 1), 15 B-cell lymphoid tumor (lanes 2-16) and 3 AML (lanes 17-19) cell lines were subjected to MSR-PCR. The B-cell lymphoid cell lines are derived from B-ALL (lanes 2-4), CLL (lanes 5-7), MCL (lane 8), FL (lane 9), DLBCL (lane 10), BL (lanes 11-12), and PCM (lanes 13-16) (Table 1). The AML cell lines (lane 17-19) were used as controls. DLC-1A methylation (160 bp) and internal control β-actin-A (257 bp) are shown in upper panel. Methylation of PCDHGA12 (310 bp) and RPIB9 (204 bp) are shown in middle and lower panels, respectively. (B) Triple markers of DNA methylation were assessed with a multiplex MSR-PCR in 29 B-ALL diagnostic bone marrow aspirates. Lane M: 100 bp DNA ladder; Lanes C1-C4: normal male (lanes 1 and 2) and female (lanes 3 and 4) blood DNA without (lanes 1 and 3) and with digestion (lanes 2 and 4); Lanes C5 and C6, positive controls using DNA from NALM-6 and M. Sss I-treated DNA; lane W: water; lanes 1-29: B-ALL patient samples; lanes N1-N4: normal individual bone marrow samples. Corresponding DNA methylation bands of 3 markers and internal control β-actin-A are denoted with arrows on the left side of the gel. (C) Peripheral blood samples from a cohort of 28 B-ALL patients at initial diagnosis (lanes B1-B28) and 4 normal individuals (lanes NB1-NB4) were subjected to MSR-PCR. Lane C1 and C2: normal human DNA without and with enzymes; lane C3 and C4: digested NALM-6 DNA and M. Sss I-treated DNA as positive controls; lane C5: water control.

FIG. 4 shows the validation of MSR-PCR method for the correlation of DLC-1 methylation with clinical follow-up in 4 B-ALL patients up to 10 years. (A) DNA from bone marrow and/or blood samples collected at multiple time points from the same patient are subjected to MSR-PCR. Controls (lanes 1-4) were normal male blood cell DNA without and with digestion, NALM-6 cell line and M.SssI-treated DNA, respectively. Lane 5 was PCR water control. In patient samples, M denotes bone marrow; Ms, bone marrow slide; B, blood; Underlined M and B indicate that the bone marrow and blood samples were collected from the same patient at the same time. (B) Correlation of DLC-1 methylation and clinical status during the period of patient follow-up (Y axis, patients; X axis, time course). Rectangles above the lines denote DLC-1 DNA methylation status; Ovals below the linen denote clinical status. Solid color indicates DNA methylation positive or patient was at diagnosis or relapsed; Empty shape indicates DNA methylation negative or patient was in remission. The positions of rectangle/oval indicate the time points of sample collection at diagnosis (the first one) and during follow-up visits.

FIG. 5 illustrates the development of a TaqMan probe-based real-time MSR-PCR (qtMSR-PCR) method. (A) The standard curve of DLC-1 CpG island assay using DLC-1Q1 primers and TaqMan probe (Table 3), the linearity ranged from 10 to 10⁸ copies per reaction with a R² value of 0.994 was obtained. (B) The distribution of the copy number of methylated DLC-1 CpG island DNA in 40 B-ALL bone marrow samples by qtMSR-PCR method. Positive controls (circled) included digested M Sss I-treated normal male human DNA and NALM-6 cell line DNA, and non-digested normal male DNA; Negative controls (circled) included digested normal male and female human DNA. The copy number was calculated with the average of triplicate samples against the standard curve in (A).

FIG. 6 illustrates the development of a SYBR Green fluorescence-based real-time MSR-PCR (qsMSR-PCR) method. Melting curves of the DLC-1Q1 primer set in control samples to confirm the specificity of amplification. Positive controls circled in red include digested SssI methylase-treated normal male and female blood genomic DNA, non-digested normal male and female blood genomic DNA. Negative controls circled in blue include digested normal male and female blood genomic DNA. This result indicates that only methylated DNA, but not normal human blood DNA, is specifically amplified by qsMSR-PCR after digestion.

FIG. 7 illustrates the development of a SYBR Green fluorescence-based real-time MSR-PCR (qsMSR-PCR) method: Standard curve. To generate the standard curve, nearly whole CpG island of DLC-1 gene was amplified using DLC-1W primers (Table 3) in GoTaq Polymerase 2× green master mix (Promega, Madison, Wis.). The PCR fragment was then purified with DNA Clean and Concentrator-5 (Zymo Research, Orange, Calif.), quantified with NanoDrop 1000 spectrophotometer, converted to copy number and used as template. The template was diluted from 10⁹ copies to 1 copy per reaction at a dilution factor of 10 and then amplified with DLC-1Q1 primers by qsMSR-PCR. Duplicate samples were used. The amplification chart is shown and a standard curve was constructed with linear regression by build-in software of iQ5 in FIG. 8.

FIG. 8 illustrates the development of a SYBR Green fluorescence-based real-time MSR-PCR (qsMSR-PCR) method: Standard curve. A broad linear range from 10 to 10⁹ copies per reaction with a R² of 0.997 was obtained. Thus the lower detection limit (sensitivity) of this method is 10 copies per reaction. This method, therefore, can be used to quantify specific DNA methylation in tumor cells.

FIG. 9 illustrates a validation of qsMSR-PCR method using DLC-1Q1 primers in detection of circulating tumor cells using DLC-1 methylation as a biomarker in a total of 94 random blood samples of cancer patients. The blood samples were obtained from a cancer center with a proved IRB protocol. Ten out of 94 samples were positive in that all 10 patients have been confirmed to have active hematopoietic or metastatic solid tumors clinically. This result indicates that the developed qsMSR-PCR method can detect CTCs and circulating tumor cell DNA.

FIG. 10 illustrates the melting curve of DLC-1 amplification in FIG. 9. Only a single peak was observed at 93° C. in the positive sample indicating the specific amplification.

### DETAILED DESCRIPTION OF INVENTION

According to certain embodiments, disclosed herein are methods useful for detection of the circulating tumor cells (CTCs) and tumor cell DNA utilizing the tumor-specific hypermethylation loci as biomarkers with either a TaqMan probe or SYBR Green flourescence-based real-time PCR technology. The present disclosure is developed upon the Applicants' detection methodology described in United States Patent Application Publication Number 2010/0248228, which is incorporated by reference in its entirety. According to the Applicants' prior application, the cancer cell detection method based on abnormal CpG hypermethylation may contain three sequential steps: 1) DNA isolation and extraction, 2) DNA digestion with pre-selected methylation sensitive enzymes, and 3) PCR process with specific primers. The present disclosure describes a method utilizing the real-time PCR process and identifies additional tumor-specific methylatation biomarkers. The prior detection method detects DNA methylation without the conventional bisulfite treatment using multiple pre-selected methylation sensitive restriction enzymes in clinical setting, Multiple Methylation Sensitive Enzyme Restriction PCR (MSR-PCR), whereas the present invention employing real-time PCR technology with expanded biomarkers is Taqman probe-based real-time PCR (qtMSR-PCR) and SYBR Green flourescence-based real-time PCR (qsMSR-PCR). Since the platform is a real-time PCR, the method is quantitative in nature.

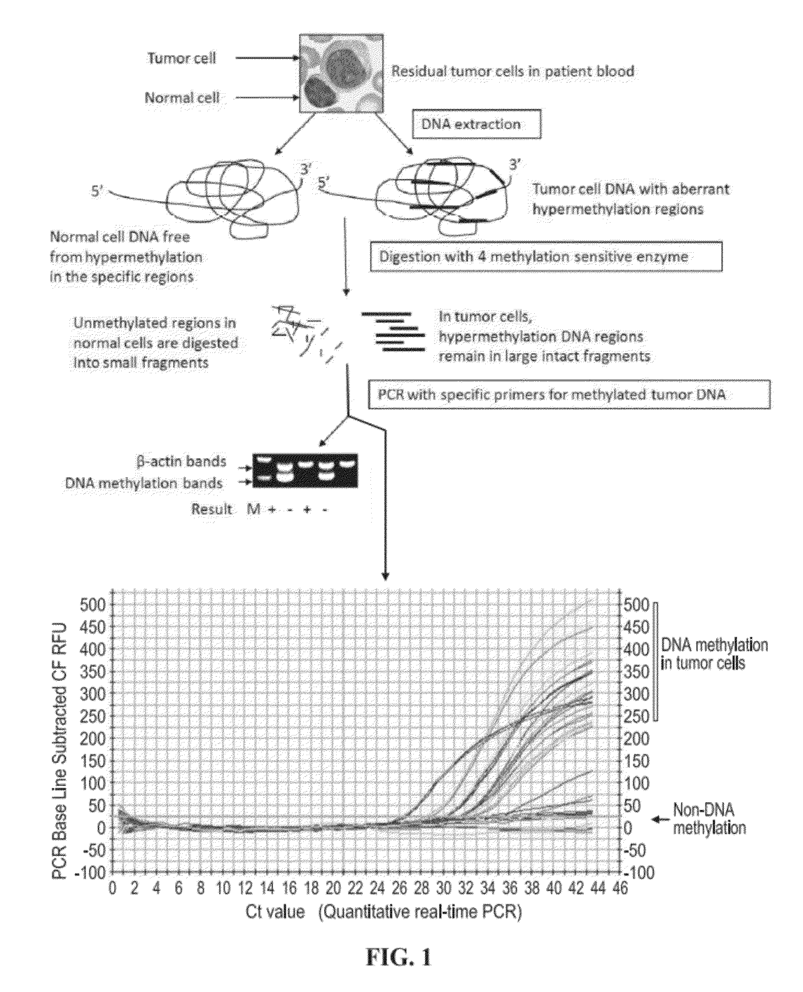

FIG. 1 illustrates the general detection method, MSR-PCR, upon which the present invention has been developed. As shown in FIG. 1, genomic DNA extracted from patients' peripheral blood is digested with four methylation sensitive enzymes. To ensure a complete digestion, multiple methylation-sensitive enzymes with four base restriction sites are selected to increase the frequency of cut sites. Specific hypermethylated regions in tumor cells are resistant to digestion, and are subsequently amplified by PCR. The same regions in normal blood or bone marrow cells are digested into small fragments and cannot be amplified. Thus, the PCR products (bands on the gel or amplification curves) represent the tumor cell, but not normal cell, population in the specimens. A restriction site-free region of the house-keeping gene β-actin is co-amplified as a PCR internal control. Multiple methylation sensitive enzymes and PCR target regions with maximal restriction sites are carefully selected within each target region to ensure a complete digestion to prevent false positive result. Lane 1 labeled as M on the gel of the right bottom indicates molecular marker; lane 2, positive control with M SssI methylase-treated normal human blood cell DNA; lane 3, negative control with pooled normal human blood DNA; lanes 4 and 5, patient samples with and without tumor cells. The amplification chart at the left bottom illustrates an example of qtMSR-PCR.

A total of 118 human genomic loci have been examined. Forty cancer specific DNA hypermethylation loci have been identified by the present disclosed method, either in MSR-PCR or qMSR-PCR or both formats. These markers include the genes of HOXD10, COX2, KLF4, SLC26A4, DLC-1, PCDHGA12A, RPIB9, SOX2, CXCR4, HIN1, SFRP2, DAPK1, CD44, CDH1, PGRB, OLIG2, NOR1, SOCS1, RECK, MAFB, p15, HOXD11, HOXA11, HOXA6, HOXA7, HOXD9, HOXA9, HOXC4, PCDHA13, HIC1, CDH13, HOXA4, PCDHA6, PCDHB15, PTPN6, APC, GSTP1, ADAM12, p16, and GABRBA. Each DNA methylation locus is found to be positive in at least one or more cancer types of cell lines and/or patient samples. The cancer cell lines used in this study include B-cell acute lymphoblastic leukemia (NALM-6, MN-60, SD1, CALL3), T-cell acute lymphoblastic leukemia (Jurkat); chronic lymphocytic leukemia (Mec 1, Mec 2, Wac-3), follicular lymphoma (RL and SC-1); mantle cell lymphoma (Granta); Burkitt lymphoma (Daudi and Raji), diffuse large B-cell lymphoma (DB); acute myeloid leukemia (KG-1, KG-1a, and Kasumi-1), breast cancer (MCF7, T-47D, HTB-26D), lung cancer (NC1-H69, NCI-H1395), colon cancer (HT-29), ovarian cancer (OVCA433 and DOV13), prostate cancer (PC-3, LNCaP), and melanoma (SK-MEL-1). Some of these cell lines are listed in Table 1.

Biomarker HOXD10 can be used in detection of several hematopoietic tumors, such as B-cell acute lymphoblastic leukemia, T-cell acute lymphoblastic leukemia, chronic lymphocytic leukemia, follicular lymphoma; mantle cell lymphoma; Burkitt lymphoma, diffuse large B-cell lymphoma, acute myeloid leukemia. It can also be used in detection of several carcinoma, such as breast cancer, lung cancer, colon cancer, ovarian cancer, prostate cancer. In addition, it can be used in detection of melanoma.

Biomarker COX 2 can be used in detection of several hematopoietic tumors, such as B-cell acute lymphoblastic leukemia, T-cell acute lymphoblastic leukemia, chronic lymphocytic leukemia, follicular lymphoma, Burkitt lymphoma, diffuse large B-cell lymphoma, and multiple myeloma. It can also be used in detection of several carcinoma, such as breast cancer and prostate cancer. In addition, it can be used in detection of melanoma.

Biomarker KLF4 can be used in detection of several hematopoietic tumors, such as B-cell acute lymphoblastic leukemia, T-cell acute lymphoblastic leukemia, multiple myeloma, acute myeloid leukemia, Diffuse large B-cell lymphoma, and Burkitt lymphoma. It can also be used in detection of carcinoma, such as ovarian cancer.

Biomarker SLC26A4 can be used in detection of several hematopoietic tumors, such as B-cell acute lymphoblastic leukemia, T-cell acute lymphoblastic leukemia, chronic lymphocytic leukemia, follicular lymphoma, mantle cell lymphoma, Burkitt lymphoma, diffuse large B-cell lymphoma, multiple myeloma, and acute myeloid leukemia. It can also be used in detection of several carcinoma, such as colon cancer and ovarian cancer.

Biomarker DLC-1 can be used in detection of several hematopoietic tumors, such as B-cell acute lymphoblastic leukemia, T-cell acute lymphoblastic leukemia, chronic lymphocytic leukemia, follicular lymphoma, mantle cell lymphoma, Burkett lymphoma, diffuse large B-cell lymphoma, and multiple myeloma. It can also be used in detection of carcinoma, such as colon cancer.

Biomarker PCDHGA12A can be used in detection of several hematopoietic tumors, such as B-cell acute lymphoblastic leukemia, T-cell acute lymphoblastic leukemia, chronic lymphocytic leukemia, follicular lymphoma; mantle cell lymphoma, Burkitt lymphoma, diffuse large B-cell lymphoma, multiple myeloma, acute myeloid leukemia, and myelodysplastic syndrome. It can also be used in detection of carcinoma, such as breast cancer, lung cancer, colon cancer, ovarian cancer, and prostate cancer. In addition, it can be used in detection of melanoma.

Biomarker RPIB9 can be used in detection of several hematopoietic tumors, such as B-cell acute lymphoblastic leukemia, T-cell acute lymphoblastic leukemia, follicular lymphoma, Burkitt lymphoma, diffuse large B-cell lymphoma, and multiple myeloma. It can also be used in detection of carcinoma, such as colon cancer.

Biomarker SOX2 can be used in detection of several hematopoietic tumors, such as B-cell acute lymphoblastic leukemia, T-cell acute lymphoblastic leukemia, diffuse large B-cell lymphoma, and Burkitt lymphoma. It can also be used in detection of carcinoma, such as colon cancer.

Biomarker CXCR4 can be used in detection of acute myeloid leukemia and colon cancer.

Biomaker HIN1 can be used in detection of B-cell acute lymphoblastic leukemia, T-cell acute lymphoblastic leukemia, multiple myeloma, acute myeloid leukemia, diffuse large B-cell lymphoma, Burkitt lymphoma, and multiple myeloma.

Biomarker SFRP2 can be used in detection of B-cell acute lymphoblastic leukemia, acute myeloid leukemia, and multiple myeloma.

Biomarker DAPK1 can be used in detection of B-cell acute lymphoblastic leukemia, acute myeloid leukemia, and multiple myeloma.

Biomarker CD44 can be used in detection of B-cell acute lymphoblastic leukemia, chronic lymphocytic leukemia, Burkitt lymphoma, and diffuse large B-cell lymphoma.

Biomarker CDH1 can be used in detection of B-cell acute lymphoblastic leukemia, acute myeloid leukemia, and Burkitt lymphoma.

Biomarker PGRB can be used in detection of B-cell acute lymphoblastic leukemia, T-cell acute lymphoblastic leukemia, acute myeloid leukemia, and multiple myeloma.

Biomarker OLIG2 can be used in detection of B-cell acute lymphoblastic leukemia and acute myeloid leukemia.

Biomarker NOR1 can be used in detection of B-cell acute lymphoblastic leukemia and acute myeloid leukemia.

Biomarker SOCS1 can be used in detection of B-cell acute lymphoblastic leukemia, acute myeloid leukemia and myelodysplastic syndrome.

Biomarker RECK can be used in detection of colon cancer.

Biomarker MAFB can be used in detection of B-cell acute lymphoblastic leukemia.

Biomaker p15 can be used in detection of acute myeloid leukemia.

Biomarker HOXD11 can be used in detection of acute lymphoblastic leukemia, chronic lymphocytic leukemia, mantle cell lymphoma, Burkett lymphoma, multiple myeloma, acute myeloid leukemia. It can also be used in detection of carcinoma, such as breast cancer, and prostate cancer.

Biomarker HOXA11 can be used in detection of carsinoma such as breast cancer and prostate cancer.

Biomarker HOXA6 can be used in detection of acute lymphoblastic leukemia, chronic lymphocytic leukemia, follicular lymphoma, mantle cell lymphoma, Burkett lymphoma, diffuse large B-cell lymphoma, multiple myeloma, and acute myeloid leukemia. It can also be used in detection of carcinoma, such as lung cancer, colon cancer, and prostate cancer.

Biomarker HOXA7 can be used in detection of carcinoma, such as breast cancer, lung cancer, colon cancer, and prostate cancer.

Biomarker HOXD9 can also be used in detection of carcinoma, such as breast cancer and prostate cancer.

Biomarker HOXA9 can be used in detection of acute lymphoblastic leukemia, chronic lymphocytic leukemia, follicular lymphoma, Burkett lymphoma, and multiple myeloma. It can also be used in detection of carcinoma, such as breast cancer, and lung cancer.

Biomarker HOXC4 can be used in detection of acute lymphoblastic leukemia, follicular lymphoma, Burkett lymphoma, multiple myeloma, and acute myeloid leukemia.

Biomarker PCDHA13 can be used in detection of carcinoma, such as breast cancer, lung cancer, and colon cancer.

Biomarker HIC1 can be used in detection of carcinoma, such as breast cancer, lung cancer, colon cancer, and prostate cancer.

Biomarker CDH13 can be used in detection of acute myeloid leukemia as well as carcinoma, such as breast cancer, ovarian cancer, and prostate cancer.

Biomarker HOXA4 can be used in detection of chronic lymphocytic leukemia.

Biomarker PCDHA6 can be used in detection of carcinoma, such as breast cancer, lung cancer, colon cancer, and prostate cancer.

Biomarker PCDHB15 can be used in detection of carcinoma, such as breast cancer, lung cancer, colon cancer, and prostate cancer.

Biomarker PTPN6 can be used in detection of carcinoma, such as breast cancer, lung cancer, colon cancer, and prostate cancer.

Biomarker APC can be used in detection of carcinoma, such as breast cancer and prostate cancer.

Biomarker GSTP1 can be used in detection of carcinoma, such as breast cancer, lung cancer, and prostate cancer.

Biomarker ADAM12 can be used in detection of breast cancer, colon cancer, acute lymphoblastic leukemia, and acute myeloid leukemia.

Biomarker p16 can be used in detection of prostate cancer, breast cancer, and colon cancer.

Biomarker GABRBA can be used in detection of prostate cancer and breast cancer.

The above mentioned and additional DNA methylation biomarkers can also be categorized by the types of tumors. For example, biomarkers to detect hematopoietic tumors can include: For acute lymphoblastic leukemia, DLC-1, PCDHGA12A, CDH1, HOXD10, RPIB9, CD44, COX2, SOX2, KLF4, SLC26A, RECK, HOXA9, HOXD11, HOXA6, ADAM12, and HOXC4; for chronic lymphocytic leukemia, DLC-1, PCDHGA12A, HOXD10, CD44, COX2, HOXA9, HOXA4, HOXD11, and HOXA6; for follicular lymphoma, DLC-1, PCDHGA12A, CDH1, HOXD10, RPIB9, COX2, KLF4, HOXA9, HOXA6, HOXC4, and SLC26A4; for mantle cell lymphoma, DLC-1, PCDHGA12A, HOXD10, HOXA9, HOXD11, and HOXA6; for Burkett lymphoma, DLC-1, PCDHGA12A, CDH1, HOXD10, RPIB9, CD44, COX2, KLF4, HOXA9, HOXD11, HOXA6, HOXC4, and SLC26A4; for diffuse large B-cell lymphoma, DLC-1, PCDHGA12A, CDH1, HOXD10, RPIB9, COX2, KLF4, HOXA6, and SLC26A4; for multiple myeloma, DLC-1, PCDHGA12A, CDH1, COX2, KLF4, HOXA9, HOXD11, HOXA6, HOXC4, HOXD10, and SLC26A; for acute myeloid leukemia, PCDHGA12A, CDH1, HOXD10, CD44, CXCR1, KLF4, SLC26A, CDH13, HOXA9, HOXD11, HOXA6, HOXC4, ADAM12, and SLC26A4; and for myelodysplastic syndrome, PCDHGA12A, SOCS-1, and HIN1.

The biomarkers for detection of carcinoma can include: For breast cancer, DLC-1, PCDHGA12A, HOXD10, RPIB9, COX2, RECK, HOXA11, HOXA7, HOXA9, HOXD9, HOXD11, PCDHB15, PCDHA6, PCDHA13, PTPN6, HIC1, CDH13, GSTP1, ADAM12, p16, GABRBA, and APC; for lung cancer, PCDHGA12A, HOXD10, HOXA7, HOXA6, HOXA9, PCDHB15, PCDHA6, PCDHA13, PTPN6, GSTP1, and HIC1; for colon cancer, DLC-1, PCDHGA12A, HOXD10, RPIB9, CD44, COX2, SOX2, CXCR1, SLC26A, RECK, HOXA7, HOXA6, HOXA9, PCDHB15, PCDHA6, PCDHA13, PTPN6, ADAM12, p16, and HIC1; for ovarian cancer, PCDHGA12A, HOXD10, SLC26A, CDH13, and RECK; and for prostate cancer, PCDHGA12A, HOXD10, COX2, HOXA7, HOXA6, HOXA9, HOXD11, HOXD9, PCDHB15, PCDHA6, PTPN6, HIC1, APC, CDH13, CDH5, HOXA11, GSTP1, p16, GABRBA, and HOXA7.

The biomarkers for detection of melanoma can include PCDHGA12A, HOXD10, KLF4, and COX2.

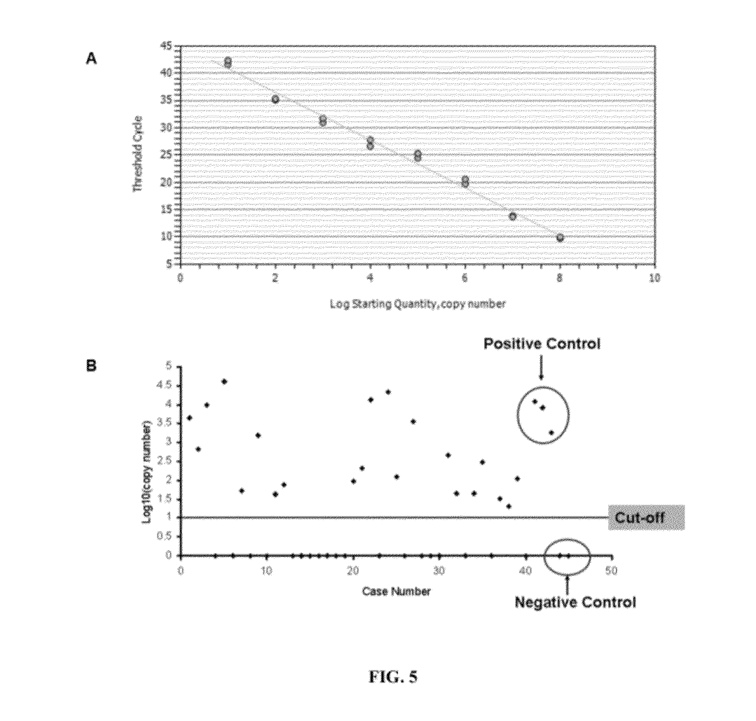

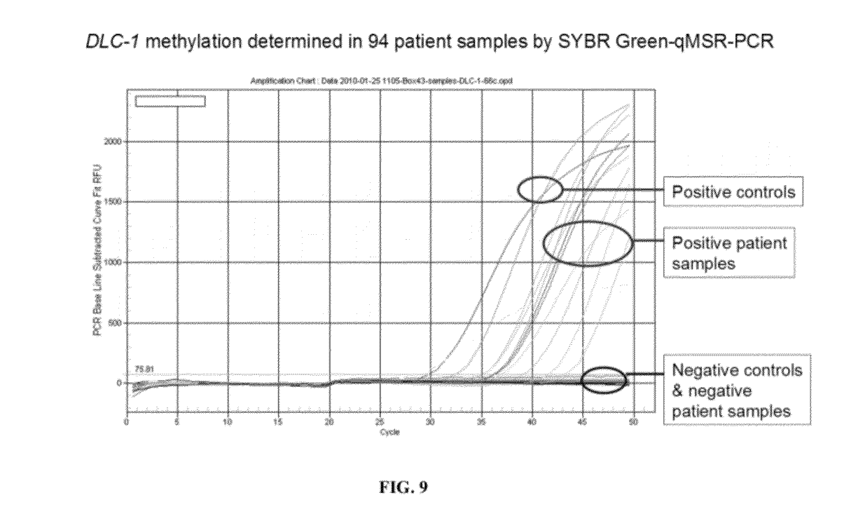

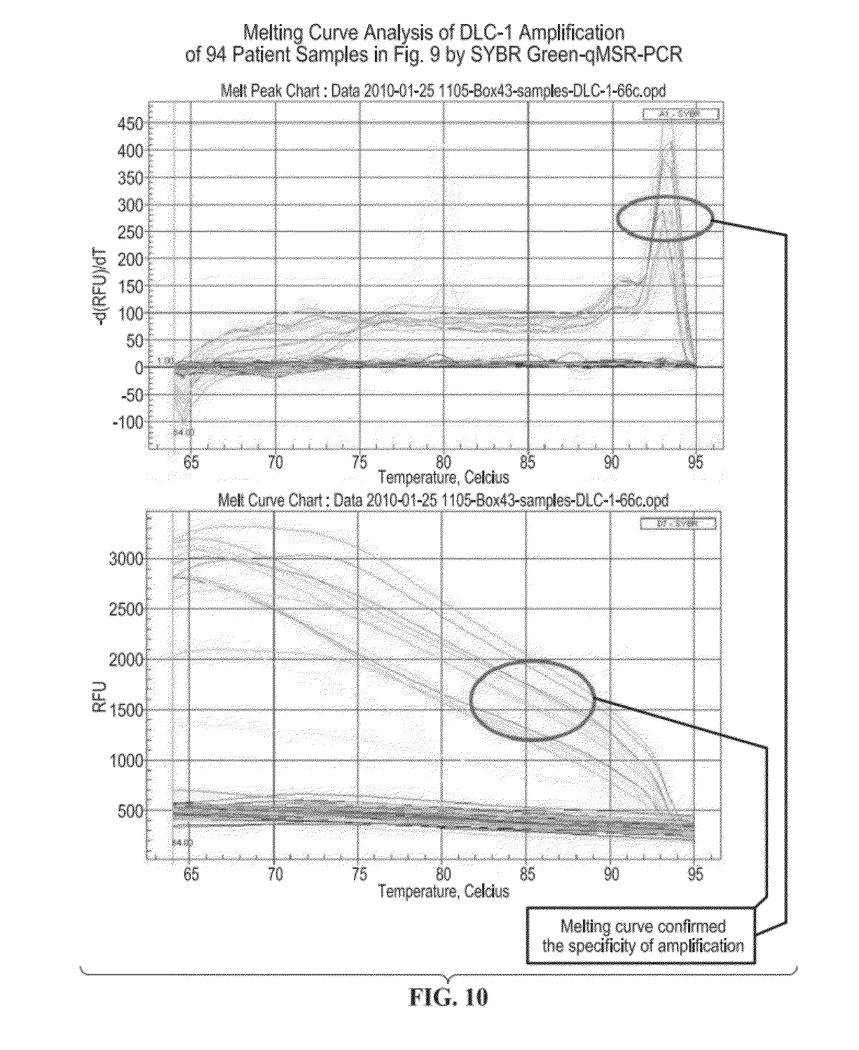

The invention further provides several exemplary procedures employing the inventive method in either conventional PCR, TaqMan probe-based real-time PCR, or SYBR Green flourescence-based real-time PCR with 3 biomarkers, DLC-1, PCDHGA12, and RPIB9 selected from the tumor-specific CGI methylation loci to detect B-cell neoplasms in a variety of B-cell lines and B lymphoblastic leukemia (B-ALL) patient blood or bone marrow specimens (FIG. 5), or cancer patient whole blood specimens (FIG. 9 and FIG. 10).

Materials and Methods

Tumor Cell Lines and Cell Line DNAs. Table 1 lists the hematopoietic tumor cell lines used in the present study. These cell lines represent a spectrum of major types of B-cell neoplasms including acute lymphoblastic leukemia, mature B-cell neoplasms, and plasma cell myeloma. All cell lines were maintained in RPMI 1640 medium supplemented with 10% FCS and 100 μg/ml of penicillin/streptomycin at standard cell culture conditions. Cells in the exponential growth phase were harvested for DNA extraction or kept at −80° C. until DNA extraction. Solid tumor cell line DNAs, including breast cancer (MCF-7, T-47D, HTB-26D), lung cancer (NC1-H69, NC1-H1395), prostate cancer (PC-3, LNCaP), colon cancer (HT-29), and melanoma (SK-MEL-1), were purchased from ATCC (Manassas, Va., USA). Ovarian cancer (OVCA433, DOV13) cell line pellets were the gift from Dr. Sharon Stack, Department of Pathology and Anatomical Sciences, the University of Missouri School of Medicine, Columbia, Mo.

TABLE 1
Summary of Cell Lines Used

| Name of | Disease entity | |
|---|---|---|
| cell line | and cell line derived | Vendors |
| NALM-6 | B lymphoblastic leukemia | DSMZ |
| MN-60 | (B-ALL) | (Braunschweig, |
| SD-1 | | Germany) |
| Jurkat | T lymphoblastic leukemia | DSMZ |
| | (T-ALL) | |
| Mec-1 | Chronic lymphocytic | DSMZ |
| Mec-2 | leukemia (CLL) | |
| Wac-3 | | |
| RL | Follicular lymphoma (FL) | ATCC |
| | with t(14; 18) | (Manassas, VA, USA) |
| Granta | Mantle cell lymphoma (MCL) | ATCC |
| | with t(11; 14) | |
| Daudi and Raji | Burkitt lymphoma (BL) | ATCC |
| DB | Diffuse large B-cell lymphoma | DSMZ |
| | (DLBCL) | |
| RPMI 8226 | Plasma cell myeloma (PCM) | ATCC |
| NCI-H929 | | |
| U266B1 | | |
| KG-1 | Acute myeloid leukemia (AML) | ATCC |
| KG-1a | | |
| Kasumi | | |
| KAS 6/1 | PCM | Dr. Jelinek, Mayo |
| | | Clinic, MN, USA |

Patient Samples and DNA Extraction. Bone marrow aspirates and peripheral blood samples were obtained from leukemia or other cancer patients at initial diagnosis as well as at follow-up visits at the Children's Hospital and Ellis Fischel Cancer Center of University of Missouri Health Care (Columbia, Mo.), the University of California at Irvine Medical Center (Irvine, Calif.) and the University of Texas Southwestern Medical Center (Dallas, Tex.) in compliance with the local Institutional Review Board (IRB) requirements. The mononuclear cell fraction from bone marrow aspirates was isolated with Ficoll-Paque Plus medium (GE Healthcare Bio-Sciences Co., Piscataway, N.J.) by gradient centrifugation and stored in liquid nitrogen until use. Peripheral blood samples in EDTA additive tubes were stored at −20° C. until use. Additionally, some bone marrow and blood smears from archived unstained slides were scraped to retrieve cells. Genomic DNA was extracted from 20 cell lines and a total of 209 clinical specimens (60 bone marrows and 149 peripheral blood samples) from 60 B-ALL patients, 105 other cancer patients and 25 healthy volunteers or non-cancer patients. Table 2 summarizes a series of 31 B-ALL clinical cases of bone marrow aspirates at initial diagnosis. Genomic DNA was isolated using the QIAamp DNA Blood mini kit (Qiagen, Valencia, Calif.). DNA concentration and purity were determined by a NanoDrop 1000 spectrophotometer (Thermo Scientific, Wilmington, Del.). Normal male and female genomic DNAs from pooled human peripheral blood were purchased from Promega (Madison, Wis.).

TABLE 2
Clinical Profile and DLC-1 Methylation Status in 31 B-ALL Patients

| | | Blast % in bone | | |
|---|---|---|---|---|
| Patients | Gender/Age | marrow | Karyotype | DLC-1 |
| 1 | M/7 | 61 | Complex | Pos |
| 2 | M/2 | 90 | Complex | Pos |
| 3 | F/10 | 79 | Complex | Pos |
| 4 | F/13 | 98 | Complex | Pos |
| 5 | M/6 | 96 | 47, XY, +21 | Pos |
| 6 | F/22 | 89 | t(9; 22)(q34; q11.2) | Neg |
| 7 | F/20 | 91 | t(4; 11), del(21) | Neg |
| 8 | M/3 | 96 | Normal | Neg |
| 9 | M/7 | 50 | N/A | Neg |
| 10 | F/4 | 77 | del(X) | Pos |
| 11 | M/3 | 86 | Normal | Neg |
| 12 | M/51 | 74 | t(9; 22)(q34; q11.2) | Neg |
| 13 | M/3 | 92 | Hyperdiploidy | Pos |
| 14 | F/84 | 95 | Normal | Pos |
| 15 | M/24 | 90 | t(2; 3), del (6) | Pos |
| 16 | M/23 | 70 | N/A | Neg |
| 17 | M/43 | 70 | Normal | Pos |
| 18 | M/49 | 90 | Normal | Neg |
| 19 | M/42 | 90 | Normal | Pos |
| 20 | M/2 | 60 | N/A | Pos |
| 21 | F/23 | 84 | N/A | Pos |
| 22 | F/11 | 90 | Hyperdiploidy | Pos |
| 23 | M/33 | 80 | N/A | Neg |
| 24 | M/20 | 50 | N/A | Pos |
| 25 | M/26 | 90 | del(Y) | Neg |
| 26 | F/15 | 64 | Normal | Pos |
| 27 | M/62 | 70 | Normal | Neg |
| 28 | M/8 | 87 | Complex | Pos |
| 29 | F/3 | 95 | Normal | Pos |
| 30 | M/6 | 94 | Normal | Pos |
| 31 | F/6 | 94 | Normal | Neg |

Note:
M: male;
F: female;
Pos: positive;
Neg: negative.
DNA methylation status of DLC-1 gene was determined by MSR-PCR in CGI region A.

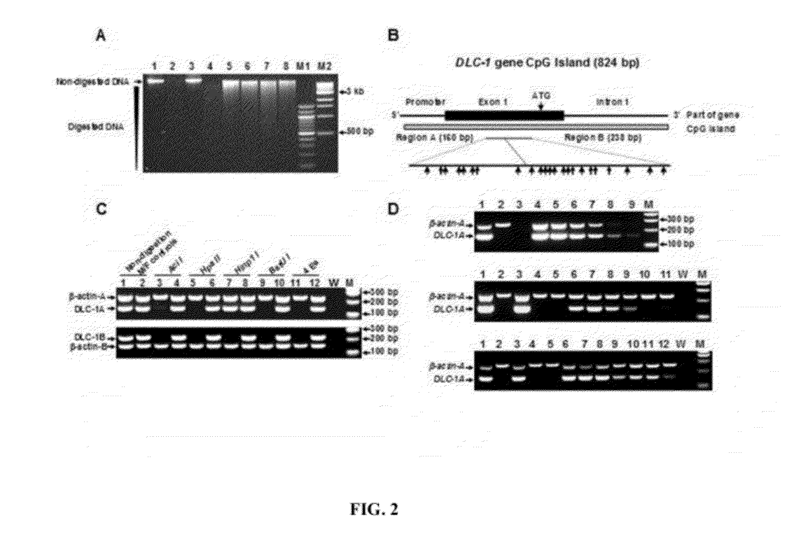

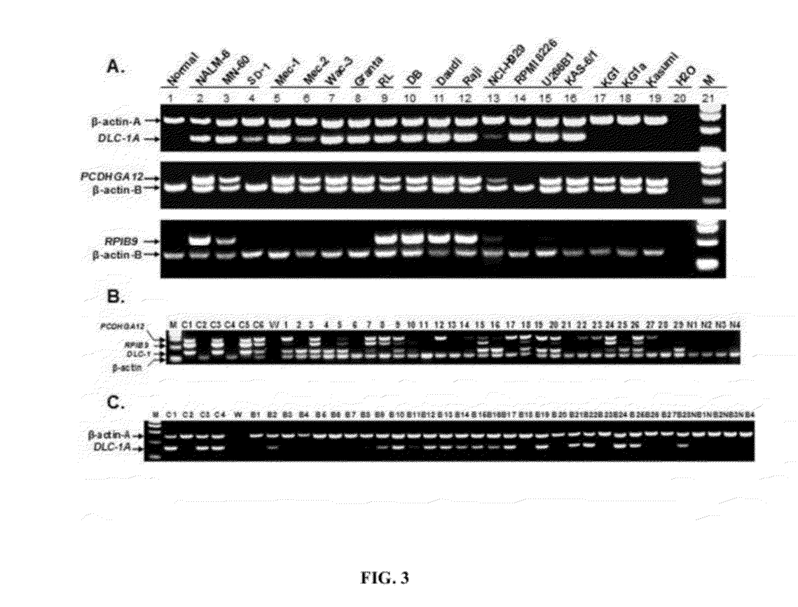

Multiple Methylation Sensitive Enzyme Restriction PCR (MSR-PCR), Quantitative Real-time Methylation Specific PCR (qMSP), Quantitative TaqMan Probe-based Real-time MSR-PCR (qtMSR-PCR), and Quantitative SYBR Green fluorescence-based Real-time MSR-PCR (qsMSR-PCR). MSR-PCR comprises three sequential steps: DNA extraction, DNA digestion and PCR (FIG. 1). To prepare methylation-positive control DNA, genomic DNA from pooled normal human blood was treated with M SssI DNA methyltransferase (New England Biolabs, Ipswich, Mass.), which methylates cytosine residues in all CG dinucleotides. In a typical digestion, the sample genomic DNA and M Sss I-treated control DNA (250 ng) were incubated with 5 U of methylation sensitive enzymes Acil, HpaII, and HinP11 (New England Biolabs, Ipswich, Mass.) in NEBuffer 4 in a final volume of 25 μl at 37° C. for 16 hours. Then 10 U of BstUI was added and digestion was continued for an additional 4 hours at 60° C. The enzymes were then inactivated at 65° C. for 20 minutes and the digested DNA was stored at −20° C. until use. In each digestion, normal human genomic DNA with and without enzymes were included as digestion controls. In a typical gel-based MSR-PCR, 40 ng of digested DNA, DLC-1 (or PCDHGA12 or RPIB9) primers (0.5 μM) and β-actin primers (0.25 μM) were mixed with GoTaq Polymerase 2× green master mix (Promega, Madison, Wis.) in a final volume of 25 μl. The PCR was carried out in a PTC100 thermal cycler (MJ Research, Ramsey, Mich.) with a program of denaturing at 95° C. for 30 seconds, annealing at 60° C. for 60 seconds, and extension at 72° C. for 60 seconds for 30 cycles with 2 minutes at 95° C. for initial denaturation and 7 minutes at 72° C. for final extension. Two sets of β-actin primers (either A or B) which amplify regions with no enzyme restriction sites in β-actin gene, were used as an internal control for the PCR. The PCR products were visualized on a 3% agarose gel containing SYBR Green 1 fluorescent dye after electrophoresis at 120 V for 30 minutes (FIG. 2C, FIG. 3).

In the nested PCR, the digested DNA was first amplified with DLC-1 primers FF/BR yielding a 383 base pair (bp) product. Then, an internal DLC-1 primer set AF/AR (160 bp) was used to amplify an aliquot of the first PCR product in the second round of PCR (FIG. 2D). Some PCR primer sequences, corresponding locations, and annealing temperatures are listed in Table 3.

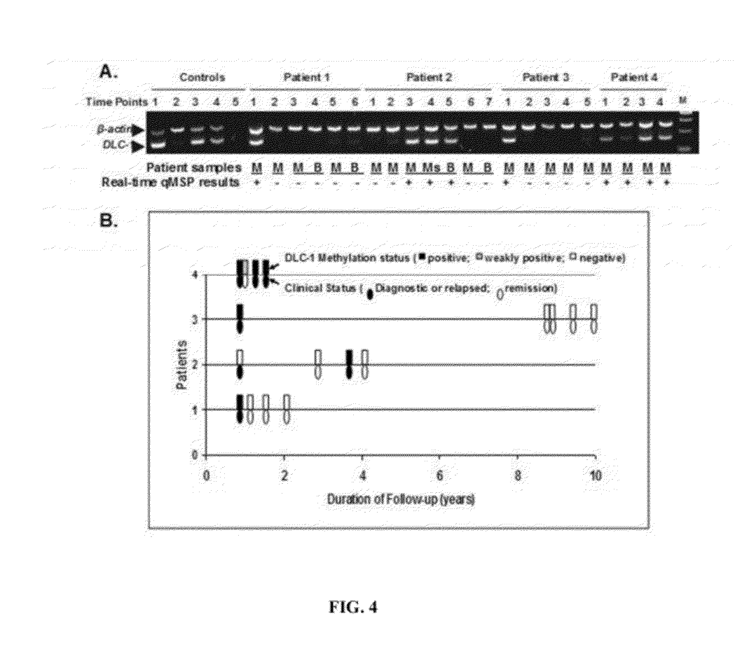

For qMSP, genomic DNA was treated with sodium bisulfite (EZ DNA methylation kit; Zymo Research, Orange, Calif.) and the real-time PCR was carried out in ABsolute QPCR mix (ABgene, Rochester, N.Y.) in a SmartCycler System (Cepheid, Sunnyvale, Calif.) as previously described [13, 14]. The sequences of primers (DLC-1Q) and probe (DLC-1Q Probe) are listed in Table 3. A positive result was defined when the ratio of DLC-1 to fl-actin signal is greater than 400. The results from MSR-PCR and qMSP were later compared on the same DNA samples in FIG. 4A.

For TaqMan probe-based qtMSR-PCR, the digested and undigested normal (digestion control) and B-ALL patient DNA samples were amplified at an iQ5 Real-time PCR detection system (BIO-RAD, Hercules, Calif.). In a typical qMSR-PCR, 20 ng of digested DNA, DLC-1Q1 primers (0.25 μM), DLC-1 TaqMan probe (0.5 μM) (IDT, Coralville, Iowa) were mixed with 2×iQ Supermix (BIO-RAD, Hercules, Calif.) in a final volume of 20 μl. The PCR program includes 3 min of denaturation at 95° C. followed by 50 cycles at 95° C. for 15 s and 60° C. for 60 s. To generate the standard curve, nearly whole CpG island of DLC-1 gene was amplified using DLC-1w primers in GoTaq Polymerase 2× green master mix (Promega, Madison, Wis.). The PCR fragment was then purified with DNA Clean and Concentrator −5 (Zymo Research, Orange, Calif.), quantified with NanoDrop 1000 spectrophotometer and used as template. The template was diluted from 10⁸ copies to 1 copy per reaction at a dilution factor of 10. The standard curve was constructed with linear regression by build-in software of iQ5 (FIG. 5A). For B-ALL patient bone marrow samples, 20 ng of digested DNA were amplified in triplicate under the same condition as negative and positive controls. The average copy number of each sample was calculated against the standard curve (FIG. 5B). Primer and probe sequences are listed in Table 3.

TABLE 3
Primer and Probe Sequences

| ID | Sequence | Orientation | Tm | SEQ ID NO |
|---|---|---|---|---|
| DLC1-AF | 5′-TAAAGAGCACAGAACAGGCACCGA-3′ | Forward | 60.4 | SEQ ID NO: 1 |
| DLC1-AR | 5′-TGCTTGATGTGCAGAAAGAAGCCG-3′ | Reverse | 60.2 | SEQ ID NO: 2 |
| DLC1-BF | 5′-TGTTAGGATCATGGTGTCCGGCTT-3′ | Forward | 60.2 | SEQ ID NO: 3 |
| DLC1-BR | 5′-AGCGCTCCCTCGTTTCGATCTTTA-3′ | Reverse | 60.2 | SEQ ID NO: 4 |
| DLC1-FF | 5′-AAATCCGGAGACTCTGCAGAAAGCG-3′ | Forward | 57.4 | SEQ ID NO: 5 |
| DLC1-WF | 5′-GAAAGTGAACCAGGGCTTCC-3′ | Forward | 61.1 | SEQ ID NO: 6 |
| DLC1-WR | 5′-TAAGGCCTGCGACCCAGA-3 | Reverse | 62.9 | SEQ ID NO: 7 |
| PCDHGA12-AF | 5′-ACTCACTTCTCCCTCATCGTGCAA-3′ | Forward | 60.1 | SEQ ID NO: 8 |
| PCDHGA12-AR | 5′-ACCTCACTTCCGCATTGACTCCTT-3′ | Reverse | 60.3 | SEQ ID NO: 9 |
| RPIB9-F | 5′-TCCAGGCTCCTTTCCTACATCCTT-3′ | Forward | 59.5 | SEQ ID NO: 10 |
| RPIB9-R | 5′-GGAGGAACCTGATC.ACCGTGT-3′ | Reverse | 61.4 | SEQ ID NO: 11 |
| b-actin-AF | 5′-GGCCGAGGACTTTGATTGCACATT-3′ | Forward | 60.2 | SEQ ID NO: 12 |
| b-actin-AR | 5′-GGGCACGAAGGCTCATCATTCAAA-3′ | Reverse | 59.9 | SEQ ID NO: 13 |
| b-actin-BF | 5′-GAGCTGGTGTCCAGGAAAAG-3′ | Forward | 59.8 | SEQ ID NO: 14 |
| b-actin-BR | 5′-GCTGGAGGATTTAAGGCAGA-3′ | Reverse | 59.4 | SEQ ID NO: 15 |
| DLC1QF | 5′-CCCAACGAAAAAACCCGACTAACG-3′ | Forward | 60.4 | SEQ ID NO: 16 |
| DLC1QR | 5′-TTTAAAGATCGAAACGAGGGAGCG-3′ | Reverse | 60.2 | SEQ ID NO: 17 |
| DLC1Q Probe | FAM/AAGTTCGTGAGTCGGCGTTTTTGA/ | | 60.8 | SEQ ID NO: 18 |
| | BHQ1 | | | |
| TaqMan Probe | FAM/CCCTCGCGGTCCTCAACGCATCCTT/ | | 73.9 | SEQ ID NO: 19 |
| | BHQ1 | | | |

Note:
ID, identification of sequences; Tm, annealing temperature of the primers and probes.

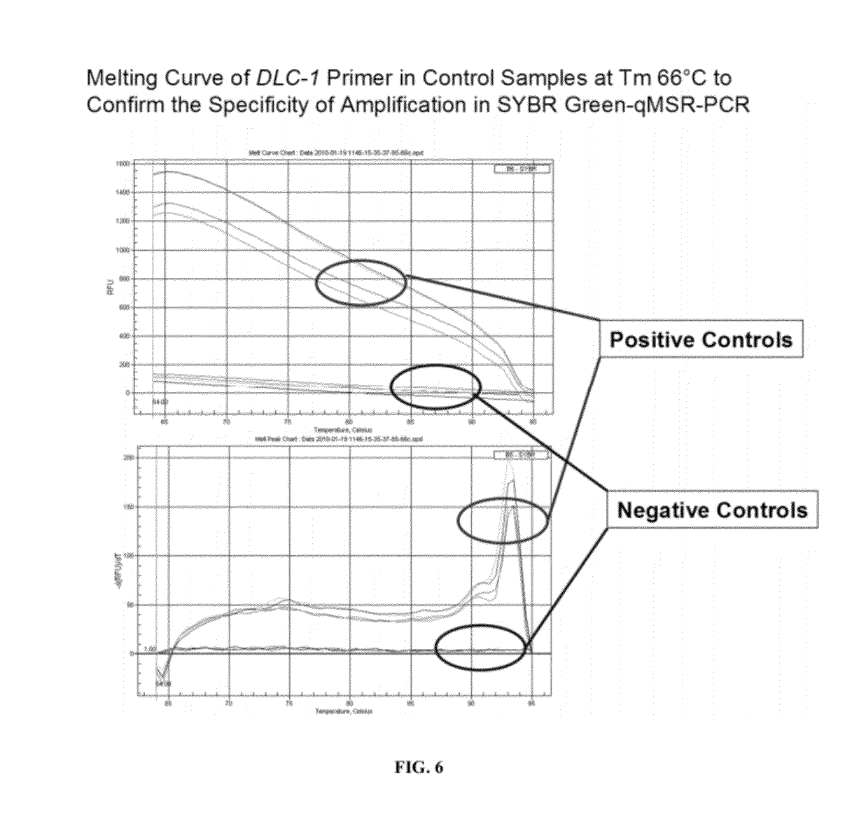

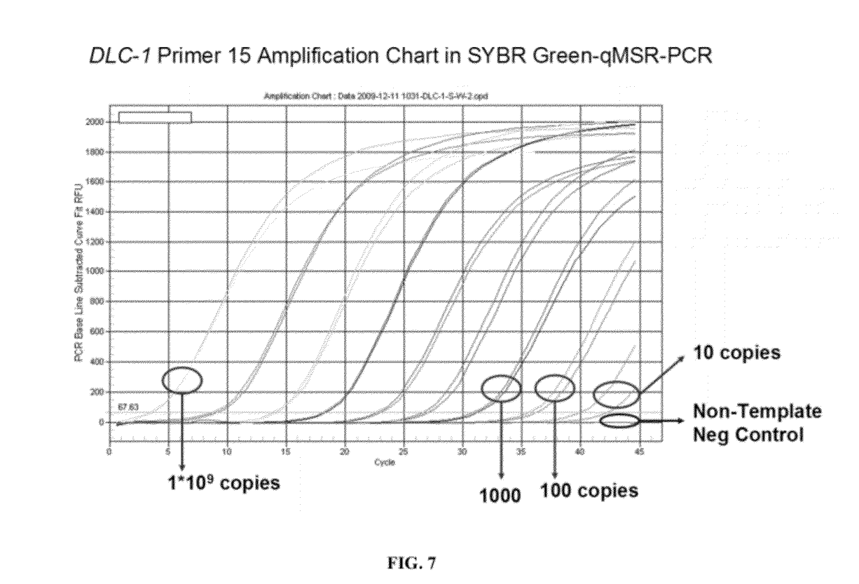

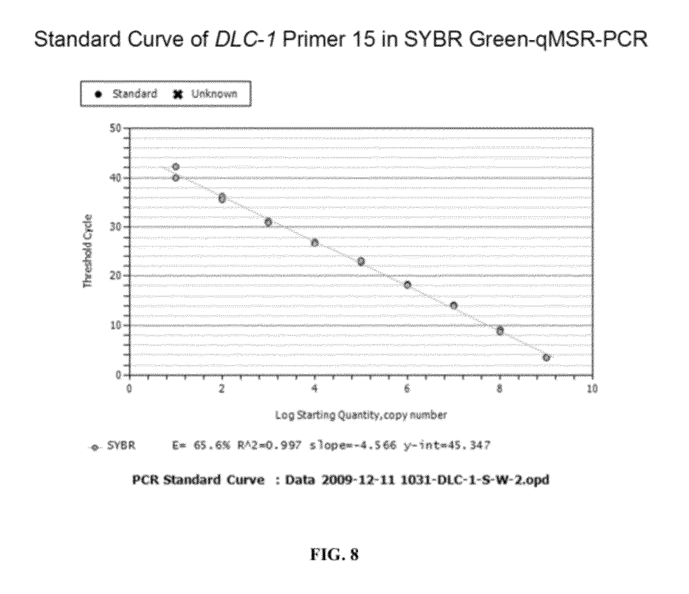

Similarly, for SYBR-green-based qsMSR-PCR, the digested DNA samples were amplified at an iQ5 Real-time PCR detection system (BIO-RAD, Hercules, Calif.). In a typical qMSR-PCR, 10 ng of digested DNA, DLC-1Q1 primers (0.25 μM each), were mixed with 10 ul of 2×SYBR Green/Fluorescein qPCR Master Mix (SABioscience, Frederick, Md.) in a final volume of 20 μl. A 2 step PCR program includes 10 min of denaturation at 95° C. (HotStart) followed by 50 cycles at 95° C. for 15 s and 64° C. for 60 s. After completion of PCR amplification, a melting curve program including 95° C. for 1 min, 64° C. for 2 min, and 64° C. to 95° C. at 2° C./min to generate melting curve (FIG. 6). To generate the standard curve, nearly whole CpG island of DLC-1 gene was amplified using DLC1W primers (Table. 3) in GoTaq Polymerase 2× green master mix (Promega, Madison, Wis.). The PCR fragment was then purified with DNA Clean and Concentrator-5 (Zymo Research, Orange, Calif.), quantified with NanoDrop 1000 spectrophotometer and converted into copy number and used as template. The template was diluted from 10⁹ copies to 1 copy per reaction at a dilution factor of 10. The standard curve was constructed with linear regression by build-in software of iQ5 (FIG. 7 and FIG. 8). For cancer patient whole blood DNA samples, 10 ng of digested DNA were amplified in duplicate under the same condition as negative and positive controls. The average copy number of each sample was calculated against the standard curve (FIG. 9). The melting curve was generated to confirm the specificity of amplification (FIG. 10).
The relative methylation level of each sample can be calculated by the delta (delta Ct) method. The same amount of M. Sss I-treated normal male human DNA was amplified as positive control and the promoter of β-actin (ACTB), without the cut site of these four enzymes in the amplified region, serve as endogenous control. After PCR reaction, the mean Ct value for the ACTB gene was subtracted from the mean Ct value of DLC-1 for each sample, using the following formula:

DLC-1ΔCt=(mean DLC-1 Ct−mean ACTB Ct)

DLC-1ΔΔCt=DLC-1ΔCt_sample—DLC-1ΔCt_Positive control

The DLC-1 relative methylation level (2^{-DLC-1ΔΔCt}×100%) was calculated for each detected sample besides the negative controls.

Results

1. Distinct DNA Methylation Patterns between Leukemic Cells and Normal Blood Cells. First, the patterns of genomic DNA methylation of acute lymphoblastic leukemia cell lines with those of normal blood samples after digestion with methylation sensitive enzymes were compared. As shown in FIG. 2A, the overall DNA methylation pattern differs between leukemia cell lines and normal blood cells. Comparing with a diffuse smear indicating much less methylation seen in normal male and female blood cell DNA (lanes 2 and 4), dense methylation in high molecular weight DNA fragments was clearly seen in all 4 leukemic cell lines (lanes 5-8). These densely methylated regions in leukemia cells might then serve as candidate biomarkers for further evaluation.

2. DCL-1, a Candidate Gene for Methylation Analysis. The genomic structure of the DLC-1 CGI, an 824 bp DNA segment encompassing the promoter region, exon 1, and part of the first intron of the gene is shown in FIG. 2B. As noted, regions A and B within the CGI were found to have many CG dinucleotides as well as multiple restriction enzyme recognition sites (10 sites in region A and 19 sites in region B), and therefore, were selected as candidate PCR targets for methylation analysis. The DNA digestion efficiency of these methylation sensitive enzymes was then examined in both regions. DLC-1 methylation in regions A (upper panel) and region B (lower panel) of the CGI were shown in FIG. 2C. Genomic DNA from normal blood samples (lanes 1, 2, 3, 5, 7, 9, 11) and B-ALL cell line NALM-6 (lanes 4, 6, 8, 10, 12) were digested with either a single enzyme or a combination, and then amplified with MSR-PCR. Methylation sensitive enzymes HpaII (lane 5) and BstUI (lane 9) gave complete digestion in both regions (no band seen) of normal blood cell DNA; Acil (lane 3) showed partial digestion (a faint band seen) in region A since only 50% digestion rate can be reached in NEBuffer 4 for this enzyme, but complete digestion was achieved in region B since more Acil restriction sites exist in that region. Hinp1I showed no digestion in region A (lane 7 of upper panel), since there is no restriction site for Hinp1I in this region. The combination of four enzymes gave complete digestion in both regions (lanes 11 in both panels) of normal blood cell DNA samples. Except lanes 3 and 7 of the upper panel of region A, in no case did normal blood DNA show cleavable amplification, but NALM-6 DNA, cut by either a single enzyme or the combined enzymes (lanes 4, 6, 8, 10, 12), was amplified. The result of differential amplification in leukemia cells, but not in normal blood cells, was encouraging, which then led us to examine the potential sensitivity of this assay.

3. Sensitivity of MSR-PCR. Analytic sensitivity can be divided into absolute and relative sensitivity [15]. Absolute sensitivity refers to the capability of detecting a minimal quantity of methylated target DNA in tumor cells. Relative sensitivity refers to the capability of detecting the smallest fraction of methylated tumor cell DNA in the presence of an excess amount of unmethylated normal cell DNA. The analytic sensitivity of MSR-PCR is shown in FIG. 2D. The upper panel demonstrates the absolute sensitivity using 80 ng of NALM-6 DNA that was digested with the combination of 4 enzymes and subsequently diluted 5-fold in a series starting from lane 4. The density of the DLC-1 methylation bands (160 bp) and β-actin-A (257 bp) bands decreased proportionately with each dilution. A weak DLC-1 methylation band was observed at 0.0256 ng of genomic DNA, equivalent to ˜5 leukemic cells (lane 9), and stronger bands at higher concentrations (lanes 4-8). Lanes 1 and 2 contain normal blood DNA with and without enzymes as digestion controls, and lane 3 contains water, instead of the DNA template, as PCR contamination control. The middle panel illustrates the relative sensitivity to detect tumor DNA at various levels mixed with normal DNA. A 10-fold serial dilution of NALM-6 DNA starting from lane 6 (250 ng NALM-6 DNA only) was mixed with normal blood DNA to make a total of 250 ng DNA (lanes 7-11). After digestion, 40 ng of the DNA mixture was amplified with MSR-PCR. A faint DLC-1 methylation band was seen with 0.25 ng of NALM-6 in 250 ng of normal DNA (lane 9) giving a relative sensitivity of 10⁻³ or 1 tumor allele in 1,000 normal cell alleles. The internal control β-actin-A band showed similar density in all lanes as expected since this gene is present in both tumor and normal cells. While this result was promising, even higher sensitivity for an effective assay to identify residual leukemic cells in clinical samples is desired. The relative sensitivity using a nested PCR was improved to 10⁻⁶, or 1 tumor cell allele in 1,000,000 normal cell alleles (lane 12 of lower panel). The density of DLC-1 bands was slightly decreased while that of β-actin bands was increased with dilution indicating a competitive effect in multiplex PCR.

4. Validation of MSR-PCR on B-cell Neoplastic Cell Lines and B-ALL Patients. After having established a sensitive detection method using a B-ALL cell line, a total of 18 leukemia cell lines (Table 1) and B-ALL patient samples is tested with two additional markers, PCDHGA12 and RPIB9 (FIG. 3). DLC-1 methylation bands were visible in all 15 B-cell tumor cell lines (lanes 2-16), although there were weaker bands (lanes 4, 6 and 13) seen in SD-1 (B-ALL), Mec-2 (CLL) and NCI-H929 (PCM) cell lines. Methylation was not seen in the normal blood cell control (lane 1) and all 3 AML cell lines KG1, KG1a and Kasumi (lanes 17-19) (FIG. 3A, upper panel). There was a similar methylation pattern for PCDHGA12 in B-cell tumor cell lines, except for SD-1 (B-ALL, lane 4) and RPMI 8226 (PCM, lane 14) (FIG. 3A, middle panel). In addition, PCDHGA12 methylation was visible in all three AML cell lines (lanes 17-19). The CGI methylation pattern of RPIB9 was very different from the other 2 genes (FIG. 3A, lower panel). Methylation was seen only in 2 B-ALL (lanes 2 and 3) and 4 mature B-cell lymphoma cell lines that are all germinal center-derived tumors (FL, DLBCL, and BL, lanes 9-12). A very weak band was also seen in a PCM cell line (lane 13).

Subsequently, clinical bone marrow aspirates from 31 B-ALL patients at initial diagnosis were examined with MSR-PCR for DLC-1 methylation. The methylation was detected in 61% (19/31) of B-ALL patients (Table 2, data not shown). CGI methylation of DLC-1, PCDHGA12 and RPIB9 was then examined in an additional 29 B-ALL bone marrow aspirates with a multiplex MSR-PCR showing a positive rate of 55% (16/29), 62% (18/29), and 31% (9/29), respectively. Taking three genes together, methylation was detected at least in one gene in 83% (24/29) of this series (FIG. 3B, lanes 1-29), demonstrating this method is capable of detecting tumor cells in the vast majority of the B-ALL cases. Methylation was not detected in either 4 normal bone marrow controls (lanes N1-N4) or pooled normal male and female blood DNA (lanes C2 and C4). The digestion controls (C1-C4), positive controls (C5-C6) and water PCR control (W) showed expected patterns.

Next, it was further examined as to whether the method may detect leukemia cells in peripheral blood samples of B-ALL patients. DLC-1 methylation was detected in 54% (15/28) of the cases (lanes B1-B28), but neither in 4 normal blood samples (lanes NB1-NB4) nor in pooled normal blood DNA (lane C2) (FIG. 3C). DLC-1 methylation was not detected in additional normal or non-cancer patient bone marrow (n=8) and blood (n=5) samples. Due to samples being collected from different locations at different times, most bone marrow aspirates and blood samples were not from the same patients. However, same DLC-1 DNA methylation pattern was seen when both bone marrow and blood samples were collected from the same patients at the same time (n=12, also in FIG. 4).

In order to develop a more sensitive and quantitative real-time PCR method (qMSR-PCR), a 763 bp fragment encompassing nearly whole region of CpG island of DLC-1 gene was amplified by PCR using DLC-1w primers. The standard curve showed an adequate linearity from 10 to 10⁸ copies per reaction (FIG. 5A). Non-template control (water) or the dilution of 1 copy per reaction was not amplified at even 45^{th} cycles. DLC-1 DNA methylation in 40 digested DNA samples of B-ALL patient bone marrows was then determined under the same conditions. When the cut-off value was set in 10 copies per reaction, 21 of 40 (52.5%) samples were positive (FIG. 5B) which is consistent with gel-based MSR-PCR method (Table 2 and FIG. 3B). The copy numbers in methylation positive patient samples calculated according to the standard curve were ranged from 20 to 39,849 copies with average of 4,592 copies per reaction.

5. Potential Use of MSR-PCR as a Tool in Monitoring B-ALL Patients. Next, it is to decide whether this method may be used to monitor the clinical course of B-ALL patients in both bone marrow and blood samples from the same patients. Bone marrow aspirates and peripheral blood samples including scraped cells from archived unstained slides (Ms) collected at different time points from 4 B-ALL patients were used. The MSR-PCR gel image along with the corresponding qMSP results is shown (FIG. 4A). A chronologic clinical course of these 4 B-ALL patients is also shown (FIG. 4B). In all cases, clinical remission or relapse was determined by a combination of bone marrow pathological examination, flow cytometry and clinical information. DLC-1 methylation as detected by qMSR-PCR and by qMSP [13, 14] on the same samples was completely concordant (FIG. 4A). The correlation between DLC-1 methylation (rectangle, above lines) and clinical status (oval, below lines) of all 4 patients was observed (FIG. 4B). As a general trend, DLC-1 methylation was positive in diagnostic and relapsed specimens, but clearly negative in specimens when patients were in remission. Interestingly, in patient 2, DLC-1 methylation was negative at initial diagnosis, but became positive at relapse after 3.2 years, and then became negative in remission after chemotherapy. In patient 4, a weak methylation band (lane 2 of FIG. 4A) was visible even though the patient had been declared a morphologic and immunophenotypic remission. Subsequently, this patient relapsed in 6 months (lanes 3 and 4). The longest follow-up time period was 10 years (patient 3). In all cases, DNA methylation status in both bone marrow and blood samples was concordant at the same time point, indicating the possible utility of using blood samples, a less invasive procedure to monitor ALL patients rather than obtaining bone marrow aspirate or biopsy.

6. Use of MSR-PCR as a Tool to Determine Hypermethylation State of Certain Marker Loci in Specific Cell Lines. Shown in Tables 4 and 5 are the results from Applicants' examination of the use of MSR-PCR to determine the hypermethylation state of marker loci in cancer cell lines. For Table 4, DNA was obtained from lung cancer cell lines (H69 and H1395), breast cancer cell lines (MCF7, MB231, and T47D), prostate cancer cell lines (LnCaP and PC3), a colon cancer cell line (HT29), and a Sss I positive cell line (positive control) and subjected to the restriction digestion and PCR analysis as described herein. The marker loci used to determine hypermethylation state for lung cancer are 213-PCDHA13, 278-PCDHGA12, 206-HOXA9, 220-PTPN6, and 277-HOXD10; for breast cancer 277-HOXD10, 278-PCDHGA12, 213-PCDHA13, 273-HOXA11, 274-HOXA7, 280-HOXA9, 202-HOXD9, and 209-PCDHB15; for prostate cancer 232-APC, 93-COX2, 220-PTPN6, 277-HOXD10, and 278-PCDHGA12; and for colon cancer 99-RECK, 213-PCDHA13, 229-CDH13, and 278-PCDHGA12. In Table 4, plus (“+”) symbols are used to designate the presence of a characteristic marker amplicon (amplified after digestions with methylation-sensitive restriction enzymes according to the real-time PCR and gel-based methods described herein). Single (“+”), double (“++”), and triple (“+++”) designations indicate the relative quantitative amount of the respective characteristic marker amplicons, respectively based on the real-time PCR and/or gel-based methods described herein.

TABLE 4
DNA hypermethylation loci in solid tumors

| | | Sss I | | | | | | | | |
|---|---|---|---|---|---|---|---|---|---|---|
| Gene | Normal | pos | H69 | H1395 | MCF7 | MB231 | T47D | LnCaP | PC3 | HT29 |
| DLC-1 | − | +++ | − | − | − | − | − | + | − | ++ |
| RPIB9 | − | + | − | − | − | − | − | − | − | + |
| SOX2 | − | ++ | − | − | − | − | +++ | − | − | ++ |
| COX2 | − | +++ | − | − | +++ | − | − | ++ | +++ | − |
| RECK | − | +++ | − | − | − | − | − | − | − | +++ |
| HOXD9 | − | ++ | + | − | +++ | +++ | +++ | + | +++ | − |
| HOXD11 | − | ++ | − | ++ | + | +++ | − | + | +++ | + |
| HOXA9 | − | ++ | +++ | ++ | +++ | ++ | +++ | − | − | − |
| PCDHB15 | + | ++++ | +++ | + | ++++ | +++ | ++++ | + | ++++ | ++ |
| PCDHA6 | + | +++ | +++ | + | +++ | ++ | +++ | ++ | +++ | ++ |
| PCDHA13 | + | ++++ | ++++ | ++++ | ++++ | +++ | ++++ | − | − | ++++ |
| PTPN6 | − | +++ | +++ | ++ | +++ | ++ | ++ | ++ | +++ | ++ |
| HIC1 | + | +++ | ++ | +++ | +++ | ++ | ++ | ++ | ++ | ++ |
| GSTP1 | − | ++ | − | + | +++ | − | − | ++ | − | − |
| GABRBA | ++ | ++++ | + | + | +++ | + | + | + | +++ | + |
| CDKN2A | − | +++ | − | − | − | − | + | − | ++ | + |
| CDH13 | − | +++ | − | − | +++ | +++ | − | − | +++ | +++ |
| APC | − | +++ | − | − | +++ | − | − | +++ | +++ | − |
| HOXA11 | − | +++ | − | − | ++++ | +++ | +++ | ++ | − | − |
| HOXA7 | − | +++ | +++ | + | ++++ | +++ | +++ | ++ | − | ++ |
| HOXA6 | − | +++ | +++ | + | +++ | + | ++ | ++ | + | + |
| HOXD10 | − | ++++ | ++++ | ++ | ++++ | ++++ | +++ | ++ | +++ | ++ |
| PCDHGA12 | + | ++++ | ++++ | ++++ | ++++ | ++++ | ++++ | ++ | ++++ | ++++ |
| HOXA9 | − | +++ | +++ | − | +++ | +++ | +++ | +++ | − | ++ |

For Table 5, DNA was obtained from ALL, AML, and MM cell lines and subjected to the restriction digestion and PCR analysis as described herein. The marker loci used to determine hypermethylation state for ALL, AML, and MM are HOXD10, COX2, KLF4, SLC26A4, DLC-1, PCDHGA12A, RPIB9, SOX2, HIN1, SFRP2, DAPK1, CDH1, PGRB, OLIG2, NOR1, SOCS1, MAFB, p15, HOXD11, HOXD10, HOXA9, HIC1, CDH13, GSTP1, and GABRBA. In Table 5, the presence or absence of a characteristic marker amplicon (amplified after digestions with methylation-sensitive restriction enzymes according to gel-based methods described herein) is designated as “−” or “+”, respectively.

TABLE 5
DNA Hypermethylation Loci in Hematopoetic cell lines by MSR-PCR

| | Normal | | | | | | | | | | |
|---|---|---|---|---|---|---|---|---|---|---|---|
| | control | | | | | | | | | | |
| | Blood | | | | | | | | | | |
| | cell | ALL | | | AML | | | | MM | | |
| Genes | DNA | NALM-6 | MN-60 | Jurkat | KG1 | KG1a | Kasumi-1 | RPMI8226 | NCI-H929 | U266B1 | KAS |
| DCL-1 | − | + | + | + | − | − | − | + | + | + | + |
| RPIB9 | − | + | + | | − | − | − | − | − | − | − |
| CDH1 | − | + | + | | − | + | + | − | − | − | − |
| PCDHGA12 | − | + | + | | + | + | + | − | + | + | + |
| p15 | − | − | | | − | + | + | − | − | − | − |
| CDH13 | − or | + | | | + | + | + | + | + | + | + |
| | weakly + | | | | | | | | | | |
| DAPK1 | − | + | | | − | + | − | − | − | − | + |
| PGRB | − | + | | | − | + | + | − | − | − | + |
| HOXD10 | − | + | | | + | + | + | − | − | − | + |
| NOR1 | − | + | | | + | − | + | − | − | − | − |
| OLIG2 | − | + | | | + | + | + | − | − | − | − |
| MAFB | − | + | | | − | − | − | | − | − | − |
| HIC1 | − or | + | | | + | + | + | | + | + | + |
| | weakly + | | | | | | | | | | |
| KLF4 | − | + | | + | − | + | + | | − | − | + |
| SOX2 | − | + | | + | − | + | − | | + | + | − |
| GSTP1 | − | − | | − | − | − | − | | − | − | − |
| SOCS1 | − | + | | + | − | + | − | − | − | − | − |
| SFRP2 | − | + | | + | − | + | − | − | − | − | + |
| HIN1 | − | + | | + | + | − | + | − | − | − | + |
| HOXA9 | − or | + | | + | − | + | + | − | − | − | + |
| | weakly + | | | | | | | | | | |
| CDH13 | − or | + | | + | + | − | + | + | + | + | + |
| | weakly + | | | | | | | | | | |
| SLC26A4 | − | + | | + | − | + | − | + | + | + | + |

Note:
ALL: Lymphocytic acute leukemia; AML: acute myeloid leukeima; MM: multiple myeloma.

Sequences of Primers and CpGs for Marker Genes. The sequences can also be found at the website http://genome.ucsc.edu/.

HOXD10
a. Primers
HOXD10F: TAGCCCCAAGGGATCTTTCC
HOXD10R: CACGGACAACAGCGACATCT
Amplicon
b. CpG island (chr2: 176982108-176982402)
CGTGGCGCGGCCAAGCCGCAGCTCTCCGCTGCCCAGCTGCAGATG
GAAAAGAAGATGAACGAGCCCGTGAGCGGCCAGGAGCCCACCAA
AGTCTCCCAGGTGGAGAGCCCCGAGGCCAAAGGCGGCCTTCCCGA
AGAGAGGAGCTGCCTGGCTGAGGTCTCCGTGTCCAGTCCCGAAGT
GCAGGAGAAGGAAAGCAAAGGTCGGTATGAGCAGAGTTGCCACCC
CAGCGGGGCGCGCAGCCCGGGAACCCGGCAGAGAGGGAGTGCCG
GGGTGCCCAGCGCCGAGCCGGAGCCCG
COX2
a. Primers
COX2-F: TTTCTTCTTCGCAGTCTTTGCCCG
COX2-R: ACGTGACTTCCTCGACCCTCTAAA
b. Amplicon
c. CpG island: Position: chr1: 186649311-
186650081; Band: 1q31.1; Genomic Size: 771
CGGAAACTCTGCCCGGGTGCGTGGAACCGGAGTCCCCGGTGCGCG
GCGCCAGGTACTCACCTGTATGGCTGAGCGCCAGGACCGCGCACA
GCAGCAGGGCGCGGGCGAGCATCGCAGCGGCGGGCAGGGCGCGG
CGCGGGGGTAGGCTTTGCTGTCTGAGGGCGTCTGGCTGTGGAGCTG
AAGGAGGCGCTGCTGAGGAGTTCCTGGACGTGCTCCTGACGCTCA
CTGCAAGTCGTATGACAATTGGTCGCTAACCGAGAGAACCTTCCTT
TTTATAAGACTGAAAACCAAGCCCATGTGACGAAATGACTGTTTCT
TTCCGCCTTTTCGTACCCCCCACAAATTTTTCCCTCCTCTCCCCTTA
AAAAAATTGCGTAAGCCCGGTGGGGGCAGGGTTTTTTACCCACGG
AAATGAGAAAATCGGAAACCCAGGAAGCTGCCCCAATTTGGGAGC
AGAGGGGGTAGTCCCCACTCTCCTGTCTGATCCCTCCCTCTCCTCCC
CGAGTTCCACCGCCCCAGGCGCACAGGTTTCCGCCAGATGTCTTTT
CTTCTTCGCAGTCTTTGCCCGAGCGCTTCCGAGAGCCAGTTCTGGA
CTGATCGCCTTGGATGGGATACCGGGGGAGGGCAGAAGGACACTT
GGCTTCCTCTCCAGGAATCTGAGCGGCCCTGAGGTCCGGGGGCGC
AGGGAATCCCCTCTCCCGCCGCCGCCGCCGTGTCTGGTCTGTACGT
CTTTAGAGGGTCGAGGAAGTCACGTCGGGACAGACTGGGGCG
KLF4
a. Primers
KLF4-F: AAAGTCCAGGTCCAGGAGATCGTT
KLF4-RCGCAATACAGACGCATCACCTCTT
b. Amplicon
c. CpG island: Position: chr9: 110249749-
110252660; Band: 9q31.2; Genomic Size: 2912
CGCCCCAGGGGGAAGTCGTGTGCAGCCGGCCGGTGGCCATTGCTG
AGAGGGGGTCCAGCGCCCAAGTGGGTGCACGAAGAGACCGCCTCC
TGCTTGATCTTGGGGCACGTGCGCGGCGGCCCGCCGTTGTAGGGCG
CCACCACCACCGGGTGGCTGCCGTCAGGGCTGCCTTTGCTGACGCT
GATGACCGACGGGCTGCCGTACTCGCTGCCAGGGGCGCTCAGCGA
CGCCTTCAGCACGAACTTGCCCATCAGCCCGCCACCTGGCGGCTGC
GGCTGCTGCGGCGGAATGTACACCGGGTCCAATTCTGGCCGCAGG
AGCTCGGCCACGAAGCCGCCCGAGGGGCTCACGTCGTTGATGTCC
GCCAGGTTGAAGGGAGCCGTCGGAGGGGGAGCGGACTCCCTGCCA
TAGAGGAGGCCTCCGCCCGTGCCGCCCGGCGCCACGCCCGGGTCG
TTCCCGGCCCGGATCGGATAGGTGAAGCTGCAGGTGGAGGGCGCG
CTGGCAGGGCCGCTGCTCGACGGCGACGACGAAGAGGAGGCTGAC
GCTGACGAGGACACGGTGGCGGCCACTGACTCCGGAGGATGGGTC
AGCGAATTGGAGAGAATAAAGTCCAGGTCCAGGAGATCGTTGAAC
TCCTCGGTCTCTCTCCGAGGTAGGGGCGCCAGGTTGCTACCGCCGC
AAGCCGCACCGGCTCCGCCGCTCTCCAGGTCTGTGGCCACGGTCGC
CGCCGCCAGGTCATAGGGGCGGCCGGGAAGCACTGGGGGAAGTCG
CTTCATGTGGGAGAGCTCCTCCCGCCAGCGCTGCGGGGACAGGGC
GGGAGAGACCTGTCAGTGGTGGTCCCCTGTTGCCACCCGACATACT
GACGTGCTGGCGGGCCACGCGCGACTGCACCGCCCAGACATGGGG
ACTGGTCAGGCAGGAAGCACCCGGGAACCCAGGGCGCCAGCGCTG
CAATCTCGGCCCACTCCCGGGTCGAAGAAGAGGTGATGCGTCTGT
ATTGCGGGTGTTATGTCCTGTCTGCCCAATTGCGTGTGAGCGAGCG
CCGCGGCTGGTCCCTCCCCCTCCAGGTCCCGTGGACGTCCCCGGAA
TTGGCACACCGAGGCTCTCTCGGTGCGCTCTCGCCACGGGGCCGCC
TACGCGCTAAACTCACTCTGGCCCAGCCAGTGTCTGGGGACGCGGC
CACCTCCCGCCCGGTGGCCCGAGAGCGCCCGCCCTACCGACAGCG
CGCCCGGGGACTGGTGAAGACCCGGCTTGCGCCCCAGGCGGCTCC
GCAGTGCTCGCACCACGGGCATACACAGCTGAGCCAAGGACACGG
AAGCTATCCCGGGAAGGTTGCGGAGTCCGCGCGGTGGCCGCTCCTT
ACCCTCGTTCAGTGGCTCTTGGTGACCCCAAGGCTCCGCCCGCCCC
CACCACACCCACGAAAACCCACCGGGCGTTCCCGGCGGCCCGGAG
CGATACTCACGTTATTCGGGGCACCTGCTTGACGCAGTGTCTTCTC
CCTTCCCGCCGGGCCAGACGCGAACGTGGAGAAAGATGGGAGCAG
CGCGTCGCTGACAGCCATGTCAGACTCGCCAGGTGGCTGCCTGCGA
GCAAGGCAGGGAGCGGAGACAGGAGAGTCAGGGGCGGCTTTCGG
CCGTCGTTCCGGCGCGTCCCACCGGTCCTCACCCCTCCCTGCTCCC
AGCGCCGCGCGCCTCACCTACCTCATTAATGTGGGGGCCCAGAAG
GTCCTCGGCAGCCCGAAGCAGCTGGGGCACCTGAACCCCAAAGTC
AACGAAGAGAAGAAACGAAGCCAAAACCCAAAACCCCAAATTGG
CCGAGATCCTTCTTCTTTGGATTAAATATAACTTGGAAGCGTCTTTT
TTAAAAAGTTCCTTTGTATACAAAAGTTCTTAGAAAAGTTGTAAAC
GCAAAAATAGACAATCAGCAAGGCGAGTAAGTAGGTCCGGTGGCC
GGGCTGCGCTCTCTTCCACTCAGCAGCGTCCCCCACCACTGTCGCG
GTCGCCTCGAGTGCTGCCGTGGGCGCAGGGGCTGTGGCCGGGGCG
GTGGGCGGGCGGTGCCGCCAGGTGAGACTGGCTGCCGTGGCGCGG
AGCTGCGAACTGGTCGGCGGCGCAAGGCGCGGACTCCGGTGAGTT
GTGTGGAGCGCGCGCGGCCATGGGCGCGGGCCACGGGCGGGTGGG
AGGGTGGGGGGCCAGAGGGGCGGGGGAGGGTCACTCGGCGGCTC
CCGGTGCCGCCGCCGCCCGCCACCGCCTCTGCTCCCCGCGCGCCCG
CAGACACGTTCGTTCTCTCTGGTCGGGAAACTGCCGGCCGCCGGCG
CGCGTTCCTTACTTATAACTTCCTTCGCTACAGCCTTTTCCTCCGCC
TTCTCCCATGCCCCGCCCCTCCCTTTCTTCTCTCCGCCCCCCCCGAG
GCTCCCTTCCATCGTTGCTATGGCAGCTAAATCAACAAACTCGGCG
CACGTGGGGGCGGGGGAGGGGAAGGAGGGGCGCGGGCGGGGCTG
GGCCGGGCCGTGACGCCAGCCAGGCAGCTGGCGGGCTGGAGCCGA
GCTGACGCCGGCGGCAGTGGTGTCGGCGGCGGCGGCGGCGTCCGC
CCCAGCGCGGGGCGCGAGGAACCGGGCGCAGGTTCGGTCGCTGCG
CGACCAGGGCCGTACTCACCGCCATTGTCGGCTCCCTGGGTTCGAA
GCCCGCGAAGACTGGTGGGGTCAGCGGGCGGCACGGTCACGCGTC
CGCACCCCTGCTAGCATACGCGCTTGCCGCGCTGTCTGCGCGCTGG
AGAAGAGCGCGATTATCCGCGTGACTCATCCAGCCCTCCATCTCCC
CCTCCCTCTCTGCGCTCGCAGGAGTCCGCTCTCGTCGCTCAGCGCC
AGTGCCGGTGGCGGTGCCGGCGCTCGGCCTGACCTCGCACGGTTCC
TCGCG
SLC26A4
a. Primers
SLC26A4-F: AGTAGCCGCCCACCTCTACTCTA
SLC26A4-R: AGTTAGTGGGTCCCAACGGCT
b. Amplicon
c. CpG island: Position: chr7: 107301206-
107302416; Band: 7q22.3; Genomic Size: 1211
CGTAAATAAAACGTCCCACTGCCTTCTGAGAGCGCTATAAAGGCA
GCGGAAGGGTAGTCCGCGGGGCATTCCGGGCGGGGCGCGAGCAGA
GACAGGTGAGTTCGCCCTGAAGATGCCCACACCGCCCGGCCCGGG
CTCCACTCCCGGGGAGGCCTCGAGGGTTGCGGATGGGACTCTTAA
GTGGTCACGGATCAGGTGGGCAGGGGGCAGTACAGCTTTCTTTCTG
AGACGCCGAGAGCGAACAGGCTGCTCGGAAAACAGGACGAGGGG
AGAGACTTGCTCAATAAGCTGAAAGTTCTGCCCCCGAGAGGGCTG
CGACAGCTGCTGGAATGTGCCTGCAGCGTCCGCCTCTTGGGGACCC
GCGGAGCGCGCCCTGACGGTTCCACGCCTGGCCCGGGGGTCTGCA
CCTCTCCTCCAGTGCGCACCTGGAGCTGCGTCCCGGGTCAGGTGCG
GGGAGGGAGGGAATCTCAGTGTCCCCTTCCAGCCTTGCAAGCGCCT
TTGGCCCCTGCCCCAGCCCCTCGGTTTGGGGGAGATTTCAGAACGC
GGACAGCGCCCTGGCTGCGGGCCATAGGGGACTGGGTGGAACTCG
GGAAGCCCCCAGAGCAGGGGCTTACTCGCTTCAAGTTTGGGGAAC
CCCGGGCAGCGGGTGCAGGCCACGAGACCCGAAGGTTCTCAGGTG
CCCCCCTGCAGGCTGGCCGTGCGCGCCGTGGGGCGCTTGTCGCGAG
CGCCGAGGGCTGCAGGACGCGGACCAGACTCGCGGTGCAGGGGGG
CCTGGCTGCAGCTAACAGGTGATCCCGTTCTTTCTGTTCCTCGCTCT
TCCCCTCCGATCGTCCTCGCTTACCGCGTGTCCTCCCTCCTCGCTGT
CCTCTGGCTCGCAGGTCATGGCAGCGCCAGGCGGCAGGTCGGAGC
CGCCGCAGCTCCCCGAGTACAGCTGCAGCTACATGGTGTCGCGGCC
GGTCTACAGCGAGCTCGCTTTCCAGCAACAGCACGAGCGGCGCCT
GCAGGAGCGCAAGACGCTGCGGGAGAGCCTGGCCAAGTGCTGCAG
GTAGCGGCCGCGCGGGCCTGCGTAGAGAGAAGCGGAGCGGGGCGT
CCACGCCTTGGGGAGGGAAGGGCGTCCCCAGCGGGCGAGAGTGGG
GTGCGGGCGGCGGAGCCCCTGGGCGCCAGCTGCTTCTCCCAGAGG
CCCGACTTTCGGTCTCCGGTCCTCCACGCCG
DLC-1
a. Primers
DLC-1AF: TGTTAGGATCATGGTGTCCGGCTT
DLC-1AR: AGCGCACCCTCGTTTCGATCTTTA
b. Amplicon
c. CpG island: Position: chr8: 12990091-
12990914; Band: 8p22; Genomic Size: 824
CGGTGTCGCCGCGCCCCTCGAGCCAGAGCCGCGAGCCCCCGCCCG
GCTCAAGGAGGAAAGTGAACCAGGGCTTCCCTTCACGGGTTGCGA
CCGATCCGGAGCCCGCCTGGTGCGCTGGCCCGCGGTCCCCAGGCA
AAAGGTAATCAAGAGTCACTCCTCCAAAATTCAAACTCCCTCCCCA
AACTGCGAGTCCTGCTATCCCCACACCACCTCCAAGAAAATCCGGA
GACTCTGCAGAAAGCGTTTAAAGAGCACAGAACAGGCACCGACTT
GACAAGGCGGGGTGACACTTTCTCGCGGCGGGTCCCCTCCGCAGC
CCGCTCCCGCGGCCAGCCCGACGGCAAGACGCAAGTCTAGCTTAC
GTGTTAGGATCATGGTGTCCGGCTTCTTTCTGCACATCAAGCACGG
CAGGCGGCGGCGGAAGCGCTGTGGGGAAGTCGAGGCAGGCGGAG
GCGGCTCGGCTTCCGCGTCGGGACCCACGGCGGCACCCGAGACGC
GCGCCCTCGCGGTCCTCAACGCATCCTTGCTCGCCGCTCCCTGCCC
CTCGTCACGGCCCCAGAAAGAAAGCGGGGTTTTCTAAAGATCGAA
ACGAGGGAGCGCTCAGGGAGTTGGGCGAGAAGTCCGTGAGCCGGC
GCTCCTGATGCGGAGAGGTGCGGCCATGTCCTGGCTGGGAGCGAA
GCGCCCTCGCTCGGGCAGTCGGAGCGAACTGTCTCCCGCGCGCTCC
GCCAGCCGGGCCCTCCCGCTGGGCCCACCCCCCGAGGGGCGGGGC
CAGAGCGGGCGGCACCGCCTCCTCCCCGCTGTCTGGGTCGCAGGCC
TTAGCGACG
PCDHA12
a. Primers
PCDHA12-AF3: AGTACCCCGAATTGGTGCTG
PCDHA12-AR3: TGCTTGCACTTCCATCTGGT
Amplicon
b. CpG island: Position: chr5: 140256274-
140257290; Band: 5q31.3; Genomic Size: 1017
CGTTGGTGCTGGACAGCGCCCTGGACCGCGAGAGCGTGTCGGCCT
ATGAGCTGGTGGTGACTGCGCGGGATGGGGGCTCGCCTTCGCTGTG
GGCCACGGCTAGAGTGTCCGTGGAGGTGGCCGACGTGAACGACAA
TGCGCCTGCGTTCGCGCAGCCCGAGTACACAGTGTTCGTGAAGGA
GAACAACCCGCCGGGCTGCCACATCTTCACGGTGTCGGCATGGGA
CGCGGACGCGCAGAAGAACGCGCTGGTGTCCTACTCGCTGGTGGA
GCGGCGGGTGGGCGAGCACGCACTGTCGAGCTACGTGTCGGTGCA
CGCGGAGAGCGGCAAGGTGTACGCGCTGCAGCCGCTAGACCACGA
GGAGCTGGAGCTGCTGCAGTTCCAGGTGAGCGCGCGCGACGCCGG
CGTGCCGCCTCTGGGCAGCAACGTGACGCTGCAGGTGTTCGTGCTG
GACGAGAACGACAACGCGCCGGCACTGCTGGCGACTCCGGCTGGC
AGCGCAGGAGGCGCAGTTAGCGAGTTGGTACCGCGGTCGGTGGGT
GCGGGCCACGTGGTGGCGAAAGTGCGCGCGGTGGACGCTGACTCC
GGCTATAACGCTTGGCTGTCCTACGAGTTGCAACCGGCGGCGGTCG
GCGCGCACATCCCGTTCCACGTGGGGCTGTACACTGGCGAGATCA
GCACGACACGCATCCTGGATGAGGCGGACGCTCCGCGCCACCGCC
TGCTGGTGCTGGTGAAGGACCACGGTGAGCCCGCGCTGACGTCCA
CGGCCACGGTGCTGGTGTCGCTGGTGGAGAACGGCCAGGCCCCAA
AGACGTCGTCGCGGGCCTCAGTGGGCGCTGTGGATCCCGAAGCGG
CTCTGGTGGATATTAACGTGTACCTCATCATCGCCATCTGTGCGGT
GTCCAGCCTGCTGGTGCTCACGCTGCTGCTGTACACTGCGCTGCGT
TGCTCAGCGCCGCCCACCGTGAGCCGGTGCGCGCCGGGCAAGCCC
ACGCTGGTGTGCTCCAGCGCCG
RPIB9
a. Primers
RPIB9-AF: TCCAGGCTCCTTTCCTACATCCTT
RPIB9-AR: ACACGGTGATACGGTTCCTCCTCT
b. Amplicon
c. CpG island: Position: chr7: 87256959-
87258444; Band: 7q21.12; Genomic Size: 1486
CGCTTCCGAACACGCGCGTCGAGGAGGGCGTTCCAGGACTCTGAG
GGAGCAGCCCAGCTGGACCGAGGCCGCGTCGTTCCTGGGCTTACT
ATTCCCAGACCCGGACTCCCGATTCCGGAGTCACGGCCCAGGACG
CGAAAAGACTCTACACTGGCACCACGCTCCTCCTTAGGCGGGCCGT
CAGTCCCGGGTGCGGGCTGCGCTGGAGGCTGAGGTGGGAGCGACA
TGGTGTGGAGGGGCAAGAAATGTCGGCACTAGACGCGCCAAGAAG
GAGATTCTACGAGCAATTCCCCCCTCGGGCCATTGTGTTGCTGTTT
ATTAGCCCCTGGGAGGGCGTCAGGACAAAAGGAACCCTCCTCCCT
TCTTAGTACTTAGGCCCAAGGTCGGGTGTGGGAGCCGGCGCGCTGC
TTTCTAGGCAGGCACTGAAGCTACGGCAGCCACGCAAATAGGTAT
CAGCCGTTAAAGCTTGGCTACAGGCAAGGGGGGGGCAATAGGCCC
CTGGCGCTGTGGGGCCCCGCATCCCACAATCCCCGCGGCTAGCCTG
TGTGGCTACTGGCGGCAGCTAGCGGGCTGCGAAAGCGAGCCCAGC
GTCCTTGACAGCAGCCCACGCGTCGGGGCGGGGCTTGAGCCCGCT
GCTTTAAAAGGTCCGCGCGGCCGGCCCCGCCCCTCTGGTGCCGCGA
TTGGATCCGGCGGGGGTAGCGTTGATTTGATAGGCGCAGAGAGGG
TGGGGCTGCGCACGCGAGGCCGGGGGCCTTGCCGCTGCCTCCCGG
GCTGGGGCACGAGTGGCTGCGGAGTGTGGGTGGTTGGGCGTGAGG
GGCCGACGGGCTCGCGCGCGCGCCGTCTGCTGAGGTCCCTCGGGA
AGGAGGAGAGCGCCTGACGCCGACCCGCAGGCGCAGCCCGGCAGT
CGGCGGCGCGCCGAGGGCGGAGGTGGTGCGTGCGTGCGTGTGTGT
GTGTGTGTGTGTGTGTGTGTGTGTGTGTGTGTGGAGCTCGGGTGCC
AAGGGCGAGCCGTCAGTCCCCGGGTGCGAGTCCCTGCTGTCTTCCA
CACCCTTCCTCCCTCCAGGCTCCTTTCCTACATCCTTCCCGCGCCCC
CACGGTTGCGGACCGAGCGAGAACCCCCTTAAGCAGGTGTGGGGG
GCGTGCGGGGTGGCACGAGACAAAAGGGGCACGGGGGTAAGCCC
GCCATGGCCTCCCGGAGCCTGGGGGGCCTGAGCGGGATCCGCGGC
GGTGGCGGCGGAGGCGGCAAGAAAAGCCTGAGCGCCCGCAATGCT
GCGGTGGAGAGGAGGAACCTGATCACCGTGTGCAGGTACGGCAGC
GCAGGGCGAGGGGAACCAGCCTCCCGCCGGGGCTGAGAGCTCTGG
GCTTCCGCGCGGGTCCTTGGGGGTCCCGGGCATGATGGGCTGCCGC
CCAGTGCCCCCGCCTATGTTGCGCCAGCCAAATCTGTGAGCGCGCA
GCTCCTTGGACAGGGGCCCGGGTCTGGACACCGTCG
SOX2
a. Primers
SOX2-F: ACAACATGATGGAGACGGAGCTGA
SOX2-R: GCCGGTATTTATAATCCGGGTGCT
b. Amplicon
c. CpG island: Position: chr3: 181430142-
181431076; Band: 3q26.33; Genomic Size: 935
CGCCCGCATGTACAACATGATGGAGACGGAGCTGAAGCCGCCGGG
CCCGCAGCAAACTTCGGGGGGCGGCGGCGGCAACTCCACCGCGGC
GGCGGCCGGCGGCAACCAGAAAAACAGCCCGGACCGCGTCAAGC
GGCCCATGAATGCCTTCATGGTGTGGTCCCGCGGGCAGCGGCGCA
AGATGGCCCAGGAGAACCCCAAGATGCACAACTCGGAGATCAGCA
AGCGCCTGGGCGCCGAGTGGAAACTTTTGTCGGAGACGGAGAAGC
GGCCGTTCATCGACGAGGCTAAGCGGCTGCGAGCGCTGCACATGA
AGGAGCACCCGGATTATAAATACCGGCCCCGGCGGAAAACCAAGA
CGCTCATGAAGAAGGATAAGTACACGCTGCCCGGCGGGCTGCTGG
CCCCCGGCGGCAATAGCATGGCGAGCGGGGTCGGGGTGGGCGCCG
GCCTGGGCGCGGGCGTGAACCAGCGCATGGACAGTTACGCGCACA
TGAACGGCTGGAGCAACGGCAGCTACAGCATGATGCAGGACCAGC
TGGGCTACCCGCAGCACCCGGGCCTCAATGCGCACGGCGCAGCGC
AGATGCAGCCCATGCACCGCTACGACGTGAGCGCCCTGCAGTACA
ACTCCATGACCAGCTCGCAGACCTACATGAACGGCTCGCCCACCTA
CAGCATGTCCTACTCGCAGCAGGGCACCCCTGGCATGGCTCTTGGC
TCCATGGGTTCGGTGGTCAAGTCCGAGGCCAGCTCCAGCCCCCCTG
TGGTTACCTCTTCCTCCCACTCCAGGGCGCCCTGCCAGGCCGGGGA
CCTCCGGGACATGATCAGCATGTATCTCCCCGGCGCCGAGGTGCCG
GAACCCGCCGCCCCCAGCAGACTTCACATGTCCCAGCACTACCAG
AGCGGCCCGGTGCCCGGCACGGCCATTAACG
CXCR4
a. Primers
CXCR4-F: AAACTCTCGAACTGCAGGACCCA
CXCR4-R: TAAGCGCCTGGTGACTGTTCTTGA
b. Amplicon
c. CpG island: Position: chr2: 136874087-
136875780; Band: 2q22.1; Genomic Size: 1694
CGGTCTTAAAACGAAGGCCCTTCGGTGCTTGGGGTATATTGGGCGG
GAGTGTCAGAAAATGAACAAACGGCACCTCCTCCCCCAAGCGGGC
GCTCCTCCGGTGTGTGGGTCTCTTGCCATCCTCGTGTTTATCACTTG
GCGCGTTTGGGACGTTAGGGAGCGGGGCATTTTCCTGGGTGGAGA
AGGTAACGGGGTCTGCACCCGTGGTCCTCGCCCCAAGTTTCATTTC
CTCACTCTCCCGGGTGGCTTCCCATTACCCCGCCACTGATCCAGTT
AACCCGGCCGGAGGTGGGCAGCTGGAAGCCTCCAGGCGGTGGGCA
CGCGGGGGGCCGGGTCGTCCAGCCCCGGGCCGCCGCGGCTGCCCA
CTACACCCACGCCAACCGCCCGCAAGCAGCGCTGCAGGGGCTCCG
CTGGGCGACACGCCAGGCTCTGTCCCACAGGGTGCTGGGGAGCGA
CTGGGCGGCTCCGCCGCGAGCGTCTTTGAATTGCGCGCCGCTGCAG
GAAACCAAAAACTCCCTAGCAAGAGGGTTTCAAAAGGTTTCTGGA
AACCACCGACGGTTAAACATCACAACTGGACTCGGAGAGAGCCAA
ACGGTTTCCCCACTTGCACCTGCCAGTCTTCGCGGCGGCGACCTGG
CAGCCCAGGTGCGGTCTTAACCGCCCCCGCCCCTCACCCCGTACCC
GCTCCTATCCCCGGAGCGCAAATCTCAGGGCTGGCAGCTGCGCGGT
GTCAAAGGGGAGGTCAAACCACTCCGCTGACCTCTGCACGACCCC
AAACTCTCGAACTGCAGGACCCACTCGCGGCCGTGGGGAAGAGGC
GCGCTTCGGACGGCGGGAAGGTTTTCCCCCTCAAACCCAAAGCGC
GCGGGCGGATCAACTCCTAGCTGCTGCCACCACTCGATCCCCTCAG
AGGATCGGCGCGGTGGGTCCACCCGCCTCTCCCGCCCTCTGCCTAC
TGTGCTGGGAGACTGGCACAGCTCCGTCGGCCGCACAGAGTTTAA
CAAACACGCACCCAGTGTCAAGAACAGTCACCAGGCGCTTAACCC
CGAAGTTAAAGCGGGCGCAATCTCCTCCTGGGAACTCAGCCCAGG
CACGCCGCCCTCCGCCTCTAAATTCAGACAATGTAACTCGCTCCAA
GACATCCCCGCTTCCCCAAGGAAGAGACCGGTGGTCTGAGTCCCG
AGGCAGCGCGCACGCCTTCTCTGCACTTGTGCACAGAATGTTCTTA
CGTTTGCAAACAGCGTGCAAGCCGCCGCGCGCGGCGGGACTCAAG
GGGGAGACACATGCAGCCACTGGAACGCTCTTTCCAGTCGTTTCTC
CTCGACTCACAGAGAAAAAGATTCCAATCCTGCTCCCCCCCCACCC
ACCCGCACTATATAGGCATGGTCAAGAAAACTCCTTTCGGTGACCC
TTTTTTGGAGTACGGGTACCTCCAATGTCCTGGCCGCTTCTGCCCGC
TCGGAGAGGGGCTGCGCTCTAAGTTCAAACGTTTGTACATTTATGA
CAAAGCAGGTTGAAACTGGACTTACACTGATCCCCTCCATGGTAAC
CGCTGGTTCTCCAGATGCGGTGGCTACTGGAGCACTCAGGCCCTCG
GCGTCACTTTGCTACCTGCTGCCGCAGCCAACAAACTGAAGTTTCT
GGCCGCGGCCGGACTTTTATAAAAACACGCTCCGAGCGCGGCGCA
TGCGCCG
HIN1
a. Primers
HIN1-F: GCAAGGCCACGAGGCTTCTTATAC
HIN1-RTCAGACCGCAAAGCGAAGGT
b. Amplicon
c. CpG island: Position: chr5: 180017100-
180019062; Band: 5q35.3; Genomic Size: 1963
CGAGCTGCTCTTAACCACGTTTATTGAGAGGGGCCGGGGGAAGGG
GATGGACGGTCCTCCCCGCGGCGGGGTTTTCAGCCCTCGCGGGTGG
GCAGCGTCTTGTCCTCAGGTGTAGATGCTCCAGTCTCGGCTCAGCC
AAACACTGTCAGGGCCCCCTGGAAAGCAGAAGCCGAGCTTGAGTG
CCCCCAGCCCTGCCACCAAGAACTCAGGCGGGGGCGCGGCAGCGG
CCGGCTCTGTGGGGAGCGGGAGCGGGGCGGTTCCGCTGGCGTCTC
CGGGGGACGCGCACCCGCGCGGGGCCATCTCCGCCTTCCCCGCCCC
TGCAGCTCGGATGCGCCCCACCCAGTTCCCACCCGGAGACCCGGG
CTTCTCCCAGGGACAGGGCTTGGAGGGGCAGGACGGGAAACAGCC
CTGACGTAGGGCCGGGACACCTCTGGTGCAGTTTTGAGGCTGGCCG
GGAAGGGATGCCCGCGCAGGAAGGGCACCCGGGGTGCCCACTTTA
CCAGCAGGGCCTTCAGGGCCTTCACGGCCCCCACGGCCTGGGGAC
CCAGCTCAGCCACACACTTCTGGGAGCCCTCTATGAGGTGGTTCAC
GGGGATGCCCAGGCTGCTCAGCAGGAGCTTCAGCGGGTTGAGGGT
GCCGAGGGGGTTGGCCAGGGTCCCGGCCCCGGCCTCCGCCGCCGA
CTCCAGCGCAGCGACAGGCTGGGCCACAGGCTTGGCCGAGCCCAC
TAAGAAAGCAGCAGCTGCAAGCGAACAGGGAGGGGTCACCGCCTG
CGCGCCGGGGTCCCCAGAAGGCAGGTCCAGGACGCGCCCCCGCGG
GAGGCGCCCAGGAACCGTCGCGCCCTGCCCGGCTCCCCGACCGCC
CCTCCCTCCTGCGCCGAGGCCTGCCAGGTGCGAGCCCCCGGGACAC
AGGCGGGTCTGGGGAGGCGGCCCCGCCAGGAGACGCTGCAGGGTC
ACCGGAGTGGCCTGAGGGTGGCGGAAGGACCGGTGAACTCTGTGC
AGGGTCCGGGACAGGCCCCCAAGGGAGGGGACACTCGCGCTGCGC
CTTGCAGGATGAGGAGCCGGTCTCCAGACGGGGGGCAGACGGGTG
TCCCCAGGCCAGGGGCGGCCTCCATCCCGGCACGAGGCTGGAGAC
AGCCCTGAGAGGGGGAGGCCGCGGGCTGCAGGCGCGGGGCCCCG
GGGTGGCGGAGCCCTCTGGGCGCCGGGCGAGGCTGGAAGGACCTG
GGATCCACGATCGGCGCAGGCAGCGGCGGGGGCGCAGCGGGCGCC
GAGGCCTCAGGCCCCACCGTGCGCGCCAGGAGCCCGGGGCGCTCA
CCGGAGCTGCAGGACAGGGCCACGCAGAGCCCCAGGAGGGCGGC
GAGCTTCATGGCGCGGGGGCTCGGGGCGCGCGGGGAACCTGCGGC
TGCCCGGGCAAGGCCACGAGGCTTCTTATACCCGGTCCTCGCCCCT
CCAGCGCCGGCCTCGCCCGCGCTCCTGAGAAAGCCCTGCCCGCTCC
GCTCACGGCCGTGCCCTGGCCAACTTCCTGCTGCGGCCGGCGGGCC
CTGGGAAGCCCGTGCCCCCTTCCCTGCCCGGGCCTCGAGGACTTCC
TCTTGGCAGGCGCTGGGGCCCTCTGAGAGCAGGCAGGCCCGGCCT
TTGTCTCCGCGAGGCCCACCCCGGCCCGCACCTTCGCTTTGCGGTC
TGACCCCACGCGCCCCCCTGCAGGGCTGGGCCCGGGTGAGGGGAG
CTTCCCTCGCGCCAGGGCAGGGGCGGGGGCGGCGCAGTTCCTGGC
TCCCTGGTCCCTGCCTCTGATCCCAGACCGTGGCAACGTCGGGCAC
TGGGGGTCCTCGTGGGCGCCTTCTGCGCCTGGGGAGGTGGAGGCG
CCAGGGACGATCAGGCCTCACTCCCGGCCGCCTCCCCGGCCGGGC
CACAGGCAGCCACAGTGCAAACAGAAGTGGGGCGTTTTTCTGTCTT
CGAAACTAGCCTCGACG
SFRP2
a. Primers
SFRP2-F: GCAATTGCTGCGCTTGTAGGAGAA
SFRP2-R: AGTCGCACCCAGCGAAGAGA
b. Amplicon
c. CpG island: Position: chr4: 154709513-
154710827; Band: 4q31.3; Genomic Size: 1315
CGCTGCTAGCGAGGGGGATGCAAAGGTCGTTGTCCTGGGGGAAAC
GGTCGCACTCAAGCATGTCGGGCCAGGGGAAGCCGAAGGCGGACA
TGACCGGGGCGCAGCGGTCCTTCACCTGCACGCAGAGCGAGTGGC
ATGGCTGGATGGTCTCGTCTAGGTCATCGAGGCAGACGGGGGCGA
AGAGCGAGCACAGGAACTTCTTGGTGTCCGGGTGGCACTGCTTCAT
GACCAGCGGGATCCAAGCGCCGGCCTGCTCCAGCACCTCCTTCATG
GTCTCGTGGCCCAGCAGGTTGGGCAGCCGCATGTTCTGGTATTCGA
TGCCGTGGCACAGCTGCAGGTTGGCAGGGATGGGCTTGCAATTGCT
GCGCTTGTAGGAGAAGTCGGGCTGGCCAAAGAGGAAGAGCCCGCG
CGCCGAGCCCAGGCAGCAGTGCGAGGCGAGGAAGAGCAGCAGCA
GCGAGCCAGGGCCCTGCAGCATCGTGGGCGCGCGACCCCGAGGGG
GCAGAGGGAGCGGAGCCGGGGAAGGGCGAGGCGGCCGGAGTTCG
AGCTTGTCCCGGGCCCGCTCTCTTCGCTGGGTGCGACTCGGGGCCC
CGAAAAGCTGGCAGCCGGCGGCTGGGGCGCGGAGAAGCGGGACA
CCGGGAGGACAGCGCGGGCGAGGCGCTGCAAGCCCGCGCGCAGCT
CCGGGGGGCTCCGACCCGGGGGAGCAGAATGAGCCGTTGCTGGGG
CACAGCCAGAGTTTTCTTGGCCTTTTTTATGCAAATCTGGAGGGTG
GGGGGAGCAAGGGAGGAGCCAATGAAGGGTAATCCGAGGAGGGC
TGGTCACTACTTTCTGGGTCTGGTTTTGCGTTGAGAATGCCCCTCAC
GCGCTTGCTGGAAGGGAATTCTGGCTGCGCCCCCTCCCCTAGATGC
CGCCGCTCGCCCGCCCTAGGATTTCTTTAAACAACAAACAGAGAA
GCCTGGCCGCTGCGCCCCCACAGTGAGCGAGCAGGGCGCGGGCTG
CGGGAGTGGGGGGCACGCAGGGCACCCCGCGAGCGGCCTCGCGAC
CAGGTACTGGCGGGAACGCGCCTAGCCCCGCGTGCCGCCGGGGCC
CGGGCTTGTTTTGCCCCAGTCCGAAGTTTCTGCTGGGTTGCCAGGC
ATGAGTGGGAGAGGGTGTGTGTGTGTGTGTGTGTGTGTGTGTGTGT
GTGTGTGTGTGTGTTGGGGGGCTGCGTCCCTGGTAGCCGCGTGTGC
CCTGTGATGGAGCCCGGGACCTGCCCGCCCGAGGCCGCCTCGGCG
AACTTCGTTTTCCCTCGAATCTCCAGCCACCGTTCAGCAGCCTGTC
G
DAPK1
a. Primers
DAPK1-GF: CTTGCAGGGTCCCCATT
DAPK1-GR: GGAACACAGCTAGGGAGTGAGT
b. Amplicon
c. CpG island: Position: chr9: 90112515-
90113817; Band: 9q21.33; Genomic Size: 1303
CGCCCGCGTTCCGGGCGGACGCACTGGCTCCCCGGCCGGCGTGGG
TGTGGGGCGAGTGGGTGTGTGCGGGGTGTGCGCGGTAGAGCGCGC
CAGCGAGCCCGGAGCGCGGAGCTGGGAGGAGCAGCGAGCGCCGC
GCAGAACCCGCAGCGCCGGCCTGGCAGGGCAGCTCGGAGGTGGGT
GGGCCGCGCCGCCAGCCCGCTTGCAGGGTCCCCATTGGCCGCCTGC
CGGCCGCCCTCCGCCCAAAAGGCGGCAAGGAGCCGAGAGGCTGCT
TCGGAGTGTGAGGAGGACAGCCGGACCGAGCCAACGCCGGGGACT
TTGTTCCCTCCGCGGAGGGGACTCGGCAACTCGCAGCGGCAGGGT
CTGGGGCCGGCGCCTGGGAGGGATCTGCGCCCCCCACTCACTCCCT
AGCTGTGTTCCCGCCGCCGCCCCGGCTAGTCTCCGGCGCTGGCGCC
TATGGTCGGCCTCCGACAGCGCTCCGGAGGGACCGGGGGAGCTCC
CAGGCGCCCGGGTGAGTAGCCAGGCGCGGCTCCCCGGTCCCCCCG
ACCCCCGGCGCCAGCTTTTGCTTTCCCAGCCAGGGCGCGGTGGGGT
TTGTCCGGGCAGTGCCTCGAGCAACTGGGAAGGCCAAGGCGGAGG
GAAACTTGGCTTCGGGGAGAAGTGCGATCGCAGCCGGGAGGCTTC
CCCAGCCCCGCGGGCCGGGTGAGAACAGGTGGCGCCGGCCCGACC
AGGCGCTTTGTGTCGGGGCGCGAGGATCTGGAGCGAACTGCTGCG
CCTCGGTGGGCCGCTCCCTTCCCTCCCTTGCTCCCCCGGGCGGCCG
CACGCCGGGTCGGCCGGGTAACGGAGAGGGAGTCGCCAGGAATGT
GGCTCTGGGGACTGCCTCGCTCGGGGAAGGGGAGAGGGTGGCCAC
GGTGTTAGGAGAGGCGCGGGAGCCGAGAGGTGGCGCGGGGGTGCC
ACCGTTGCCGCAGGCTGGAGAGAGATTGCTCCCAGTGAGGCGCGT
ACCGTCTGGGCGAGGGCTTCATTCTTCCGCGGCGTCCCTGGAGGTG
GGAAAGCTGGGTGGGCATGTGTGCAGAGAAAGGGGAGGCGGGGA
GGCCAGTCACTTCCGGAGCCGGTTCTGATCCCAACAGACCGCCCAG
CGTTTGGGGACGCCGACCTCGGGGTGCCGTGGTGCCCGGCCCCAC
GCGCGCGCGGGGCTGAGGGGTCGGGGGCGTCCCTGGCCGCCCAGC
TTTAACAAAGGGTGCTCCTCTCCACCCCGCGAGGAGGGGCAGCTCC
GGAGACCCGGTCTTCAGCGAGCGGGGTCTTAGCGCCG
CD44
a. Primers
CD44-F: GGAGAAGAAAGCCAGTGCGTC
CD44-R: AAACAGTGACCTAAGACGGAGGGA
b. Amplicon
c. CpG island: Position: chr11: 35160376-
35161000; Band: 11p13; Genomic Size: 625
CGGTTCGGTCATCCTCTGTCCTGACGCCGCGGGGCCAGCGGGAGA
AGAAAGCCAGTGCGTCTCTGGGCGCAGGGGCCAGTGGGGCTCGGA
GGCACAGGCACCCCGCGACACTCCAGGTTCCCCGACCCACGTCCCT
GGCAGCCCCGATTATTTACAGCCTCAGCAGAGCACGGGGCGGGGG
CAGAGGGGCCCGCCCGGGAGGGCTGCTACTTCTTAAAACCTCTGC
GGGCTGCTTAGTCACAGCCCCCCTTGCTTGGGTGTGTCCTTCGCTC
GCTCCCTCCCTCCGTCTTAGGTCACTGTTTTCAACCTCGAATAAAA
ACTGCAGCCAACTTCCGAGGCAGCCTCATTGCCCAGCGGACCCCA
GCCTCTGCCAGGTTCGGTCCGCCATCCTCGTCCCGTCCTCCGCCGG
CCCCTGCCCCGCGCCCAGGGATCCTCCAGCTCCTTTCGCCCGCGCC
CTCCGTTCGCTCCGGACACCATGGACAAGTTTTGGTGGCACGCAGC
CTGGGGACTCTGCCTCGTGCCGCTGAGCCTGGCGCAGATCGGTGAG
TGCCCGCCGCAGCCTGGGCAGCAAGATGGGTGCGGGGTGCTCAGC
GCGGACCCGGCGGCAGCCCCTCCGGCTGAGTCG
CDH1
a. Primers:
CDH1QF: TGAGCTTGCGGAAGTCAGTTCAGA
CDH1QR: TTCTTGGAAGAAGGGAAGCGGTGA
b. Amplicon
c. CpG island: Position: chr16: 68771035-
68772344; Band: 16q22.1; Genomic Size: 1310
CGCGTCTATGCGAGGCCGGGTGGGCGGGCCGTCAGCTCCGCCCTG
GGGAGGGGTCCGCGCTGCTGATTGGCTGTGGCCGGCAGGTGAACC
CTCAGCCAATCAGCGGTACGGGGGGCGGTGCCTCCGGGGCTCACC
TGGCTGCAGCCACGCACCCCCTCTCAGTGGCGTCGGAACTGCAAA
GCACCTGTGAGCTTGCGGAAGTCAGTTCAGACTCCAGCCCGCTCCA
GCCCGGCCCGACCCGACCGCACCCGGCGCCTGCCCTCGCTCGGCGT
CCCCGGCCAGCCATGGGCCCTTGGAGCCGCAGCCTCTCGGCGCTGC
TGCTGCTGCTGCAGGTACCCCGGATCCCCTGACTTGCGAGGGACGC
ATTCGGGCCGCAAGCTCCGCGCCCCAGCCCTGCGCCCCTTCCTCTC
CCGTCGTCACCGCTTCCCTTCTTCCAAGAAAGTTCGGGTCCTGAGG
AGCGGAGCGGCCTGGAAGCCTCGCGCGCTCCGGACCCCCCAGTGA
TGGGAGTGGGGGGTGGGTGGTGAGGGGCGAGCGCGGCTTTCCTGC
CCCCTCCAGCGCAGACCGAGGCGGGGGCGTCTGGCCGCGGAGTCC
GCGGGGTGGGCTCGCGCGGGCGGTGGGGGCGTGAAGCGGGGTGTA
GGGGGTGGGGTGTGGAGAAGGGGTGCCCTGGTGCAAGTCGAGGGG
GAGCCAGGAGTCGTGGGGACGATCTTCGAGGGAAGGAGAGGGGC
ATCCGTAGAAATAAAGGCACCTGCCATGCCAAGAAAGGTCGTAAA
TAGGAGTGAGGGTCCCGGGGATAAGAAAGTGAGGTCGGAGGAGGT
GGGAGCGCCCCTCGCTCTGAGGAGTGGTGCATTCCCGGTCTAAGG
AAAGTGGGGTACTGGAGAATAAAGACATCTCCAATAAAATGAGAA
AGGAGACTGAAAGGGAACGGTGGGCTAGGTCTTGAGGGGGTGACT
CGGCGGCCCCCTCCCGGGAGTTCCTGGGGGCTCGGCGGCCGTAGG
TTTCGGGGTGGGGGAGGGTGACGTCGCTGCCCGCCCGTCCCGGGG
CTGCGGGCTGGGGTCCTCCCCCAATCCCGACGCCGGGAGCGAGGG
AGGGGCGGCGCTGTTGGTTTCGGTGAGCAGGAGGGAACCCTCCGA
GTCACCCGGTTCCATCTACCTTTCCCCCACCCCAGGTCTCCTCTTGG
CTCTGCCAGGAGCCGGAGCCCTGCCACCCTGGCTTTGACGCCGAGA
GCTACACGTTCACGGTGCCCCGGCGCCACCTGGAGAGAGGCCGCG
TCCTGGGCAGAGGTGAGGGCGCGCTGCCGGTGTCCCTGGGCG
PGRB
a. Primers
PGRB-F: ATAAGGCGTGATTGAGAGGCAGGA
PGRB-R: TTGAGGAGGAGGATGGCTCTGAGT
b. Amplicon
c. CpG island: Position: chr16: 68771035-
68772344; Band: 16q22.1; Genomic Size: 1310
CGCGTCTATGCGAGGCCGGGTGGGCGGGCCGTCAGCTCCGCCCTG
GGGAGGGGTCCGCGCTGCTGATTGGCTGTGGCCGGCAGGTGAACC
CTCAGCCAATCAGCGGTACGGGGGGCGGTGCCTCCGGGGCTCACC
TGGCTGCAGCCACGCACCCCCTCTCAGTGGCGTCGGAACTGCAAA
GCACCTGTGAGCTTGCGGAAGTCAGTTCAGACTCCAGCCCGCTCCA
GCCCGGCCCGACCCGACCGCACCCGGCGCCTGCCCTCGCTCGGCGT
CCCCGGCCAGCCATGGGCCCTTGGAGCCGCAGCCTCTCGGCGCTGC
TGCTGCTGCTGCAGGTACCCCGGATCCCCTGACTTGCGAGGGACGC
ATTCGGGCCGCAAGCTCCGCGCCCCAGCCCTGCGCCCCTTCCTCTC
CCGTCGTCACCGCTTCCCTTCTTCCAAGAAAGTTCGGGTCCTGAGG
AGCGGAGCGGCCTGGAAGCCTCGCGCGCTCCGGACCCCCCAGTGA
TGGGAGTGGGGGGTGGGTGGTGAGGGGCGAGCGCGGCTTTCCTGC
CCCCTCCAGCGCAGACCGAGGCGGGGGCGTCTGGCCGCGGAGTCC
GCGGGGTGGGCTCGCGCGGGCGGTGGGGGCGTGAAGCGGGGTGTA
GGGGGTGGGGTGTGGAGAAGGGGTGCCCTGGTGCAAGTCGAGGGG
GAGCCAGGAGTCGTGGGGACGATCTTCGAGGGAAGGAGAGGGGC
ATCCGTAGAAATAAAGGCACCTGCCATGCCAAGAAAGGTCGTAAA
TAGGAGTGAGGGTCCCGGGGATAAGAAAGTGAGGTCGGAGGAGGT
GGGAGCGCCCCTCGCTCTGAGGAGTGGTGCATTCCCGGTCTAAGG
AAAGTGGGGTACTGGAGAATAAAGACATCTCCAATAAAATGAGAA
AGGAGACTGAAAGGGAACGGTGGGCTAGGTCTTGAGGGGGTGACT
CGGCGGCCCCCTCCCGGGAGTTCCTGGGGGCTCGGCGGCCGTAGG
TTTCGGGGTGGGGGAGGGTGACGTCGCTGCCCGCCCGTCCCGGGG
CTGCGGGCTGGGGTCCTCCCCCAATCCCGACGCCGGGAGCGAGGG
AGGGGCGGCGCTGTTGGTTTCGGTGAGCAGGAGGGAACCCTCCGA
GTCACCCGGTTCCATCTACCTTTCCCCCACCCCAGGTCTCCTCTTGG
CTCTGCCAGGAGCCGGAGCCCTGCCACCCTGGCTTTGACGCCGAGA
GCTACACGTTCACGGTGCCCCGGCGCCACCTGGAGAGAGGCCGCG
TCCTGGGCAGAGGTGAGGGCGCGCTGCCGGTGTCCCTGGGCG
OLIG2
a. Primers
OLIG2-F: TTTGACCACGTTCCCTTTCTCCCT
OLIG2-R: TCCGGGCTAATTCCGCTCAATGAA
b. Amplicon
c. CpG island: Position: chr21: 34395129-
34400245; Band: 21q22.11; Genomic Size: 5117
gtgggagggg tagaggaaaa gcccgcaggg gccaggttgg
gaccccgtag gccgggttag agggcttgga cttgatcctg
acaggcgaca gggagacata ttgctactta ttatgtgcac
agtggccaga tctctaaaga aaacaccatc ccccaccccc
accccccata tagtaaacca ggtggtccgc ccagtgctcc
cagggaggtg atgggaaatc ccactccata ccctgcggtg
aggggttcca tgccctccac gtgtgcaact actccgggcc
cagggaaaca ctgggcccca tccggtaacc cccggcccag
tcgggtttcc cagttcacat tataaccaaa cggtcttgcc
agctagacag acagacaccc ctgacctgtt taccctgatc
ctctgctctc aggattaatc acaacttgtc gaagggggtg
gcttccagtg gggtggaccg ctctgtcaat gccagcgtgt
gtctagcatc tcctggggtg ggggtgtggg gaagggaggt
gtaggatgaa gccctagaag cctcaggcaa ttgtgatccg
gtgggctgga tactgaagcc cacccctgcc ttgacctcaa
ttttcagtat cttcatctgt aaaatgggaa caacctgcct
tcctcctagc cctaaagggg ctgctgtcaa gattggctga
gatagctgtt tgcaagctga gctcaatgaa agttcattgt
gtccccctca gtcctatccc aatatcgtct cactgcaaag
gtggggggca gcttaacttc aagggcactt caaggatagc
caggtggctg tcagcccagc tttccaggat gggagcagga
tcttgacaga agggttgact gggaggggca gttgctggtt
tgggcttcgt taggttgcat ttttgtttgt tgtcctttca
tttccctggg gcagcacccc ttcctgcaag ctccaggcct
tcctctggaa tgctcctaga gcccaacctc tgctggtgcc
tgagcttaag ccaggccagc taaggggatc ctggattcac
acggcctcac agtcactcag attgttagca gaagacaaaa
attacaaggg gagggcgtca tgtgattctt acacaccctc
caaatccagc agacaccttg gaagccacag gtagcttcaa
gaaacccatt ttacggatga gaacctgaga tggagaaagg
acaactggag atctctgagt ctctgagccc acactcccta
cctccctgca cctccaggca ctctgctggc aggatcttgg
gcaaatgccc acagctctct gagagtcagt tttcctgtct
gtaaaatggg agtcatacct tcctcctatg gccggtgaga
gactaaatta aactatgtct gtcaagacac ctgaaactcc
tggcacaatt taggttgcct tcaagtggtc acagttgtca
ttaggtggaa gtcaacaccc caatcattgt aaaggtgccc
atatacccca agatccagat tacagctctc acagtttatt
atatacagcg aaaaaacaca taacacacct ttgcccacat
ttacatgtat tttacggacc atgtttcaca tcagtccgca
tgcacatctg cacgtgtgtg cattcggcag tatttaccaa
gcacctgcca agtgccaggg cctgtcctcc gcacccggcg
tgaactgtcc tggaccagtc ccgggagccg cggttctgac
cagccgtgct gaccctggac gactccatga gctgttttgt
gagaaagaca cgccatttgt ttgcagagtt ctgacttctg
aggggtcatg tagcacatgt ttggtagcca aacgctgtca
ttcacgacca ggagcgatgg ctgcaatgcc tttttctttg
ctttgctttc cggtgccggg agccttgcct cccgccgcca
cccctggtca gctctgcgca agaacgtcgt tctgtttggc
agccaggccg agacgcagcc tgaatgtgag caggaactcg
gagaagggaa gggagagaat cagaaagaag gcccgggagg
gacccgggaa gcagtgggag gtctgcgccc tggagccccg
cgagagcccg ccggtttggc acgggctcct cccgggccgc
ccggcggtcc aacaaaggcc ggccccgaca cgcacccggt
cttttgtggg agagaaacac aaagaagagg gaaaaacacg
gaggaggcca acagcaccag gacgcggggg ccaaccagga
actcccggag ccggggccca ttagcctctg caaatgagca
ctccattccc caggaagggg ccccagctgc gcgcgctggt
gggaaccgca gtgcctggga cccgcccagg tcgcccaccc
cgggcgccgg gcgcaggacc cggacaagtc ctggggacgc
ctccaggacg caccagggca agcttgggca ccgggatcta
atttctagtt attcctggga cggggtgggg aggcatagga
gacacaccga gaggtactca gcatccgatt ggcaccaggg
ccaagggagc ccaggggcga cacagacctc cccgacctcc
caagctactc cggcgacggg aggatgttga gggaagcctg
ccaggtgaag aaggggccag cagcagcaca gagcttccga
ctttgccttc caggctctag actcgcgcca tgccaagacg
ggcccctcga ctttcacccc tgactcccaa ctccagccac
tggaccgagc gcgcaaagaa cctgagaccg cttgctctca
ccgccgcaag tcggtcgcag gacagacacc agtgggcagc
aacaaaaaaa gaaaccgggt tccgggacac gtgccggcgg
ctggactaac ctcagcggct gcaaccaagg agcgcgcacg
ttgcgcctgc tggtgtttat tagctacact ggcaggcgca
caactccgcg ccccgactgg tggccccaca gcgcgcacca
cacatggcct cgctgctgtt ggcggggtag gcccgaagga
ggcatctaca aatgcccgag ccctttctga tccccacccc
cccgctccct gcgtcgtccg agtgacagat tctactaatt
gaacggttat gggtcatcct tgtaaccgtt ggacgacata
acaccacgct tcagttcttc atgttttaaa tacatattta
acggatggct gcagagccag ctgggaaaca cgcggattga
aaaataatgc tccagaaggc acgagactgg ggcgaaggcg
agagcgggct gggcttctag cggagaccgc agagggagac
atatctcaga actaggggca ataacgtggg tttctctttg
tatttgttta ttttgtaact ttgctacttg aagaccaatt
atttactatg ctaatttgtt tgcttgtttt taaaaccgta
cttgcacagt aaaagttccc caacaacgga agtaacccga
cgttcctcac actccctagg agactgtgtg cgtgtgtgcc
cgcgcgtgcg ctcacagtgt caagtgctag catccgagat
ctgcagaaac aaatgtctga attcgaaatg tatgggtgtg
agaaattcag ctcggggaag agattaggga ctgggggaga
caggtggctg cctgtactat aaggaaccgc caacgccagc
atctgtagtc caagcagggc tgctctgtaa aggcttagca
attttttctg taggcttgct gcacacggtc tctggctttt
cccatctgta aaatgggtga atgcatccgt acctcagcta
cctccgtgag gtgcttctcc agttcgggct taattcctca
tcgtcaagag ttttcaggtt tcagagccag cctgcaatcg
gtaaaacatg tcccaacgcg gtcgcgagtg gttccatctc
gctgtctggc ccacagcgtg gagaagcctt gcccaggcct
gaaacttctc tttgcagttc cagaaagcag gcgactggga
cggaaggctc tttgctaacc ttttacagcg gagccctgct
tggactacag atgccagcgt tgcccctgcc ccaaggcgtg
tggtgatcac aaagacgaca ctgaaaatac ttactatcat
ccggctcccc tgctaataaa tggaggggtg tttaactaca
ggcacgaccc tgcccttgtg ctagcgcggt taccgtgcgg
aaataactcg tccctgtacc cacaccatcc tcaacctaaa
ggagagttgt gaattctttc aaaacactct tctggagtcc
gtcccctccc tccttgcccg ccctctaccc ctcaagtccc
tgcccccagc tgggggcgct accggctgcc gtcggagctg
cagccacggc catctcctag acgcgcgagt agagcaccaa
gatagtgggg actttgtgcc tgggcatcgt ttacatttgg
ggcgccaaat gcccacgtgt tgatgaaacc agtgagatgg
gaacaggcgg cgggaaacca gacagaggaa gagctaggga
ggagacccca gccccggatc ctgggtcgcc agggttttcc
gcgcgcatcc caaaaggtgc ggctgcgtgg ggcatcaggt
tagtttgtta gactctgcag agtctccaaa ccatcccatc
ccccaacctg actctgtggt ggccgtattt tttacagaaa
tttgaccacg ttccctttct cccttggtcc caagcgcgct
cagccctccc tccatccccc ttgagccgcc cttctcctcc
ccctcgcctc ctcgggtccc tcctccagtc cctccccaag
aatctcccgg ccacgggcgc ccattggttg tgcgcaggga
ggaggcgtgt gcccggcctg gcgagtttca ttgagcggaa
ttagcccgga tgacatcagc ttcccagccc cccggcgggc
ccagctcatt ggcgaggcag cccctccagg acacgcacat
tgttccccgc ccccgccccc gccaccgctg ccgccgtcgc
cgctgccacc gggctataaa aaccggccga gcccctaaag
gtgcggatgc ttattataga tcgacgcgac accagcgccc
ggtgccaggt tctcccctga ggcttttcgg agcgagctcc
tcaaatcgca tccagagtaa gtgtccccgc cccacagcag
ccgcagccta gatcccaggg acagactctc ctcaactcgg
ctgtgaccca gaatgctccg atacaggggg tctggatccc
tactctgcgg gccatttctc cagagcgact ttgctcttct
gtcctcccca cactcaccgc tgcatctccc tcaccaaaag
cgagaagtcg gagcgacaac agctctttct gcccaagccc
cagtcagctg gtgagctccc cgtggtctcc agatgcagca
catggactct gggccccgcg ccggctctgg gtgcatgtgc
gtgtgcgtgt gtttgctgcg tggtgtcgat ggagataagg
tggatccgtt tgaggaacca aatcattagt tctctatcta
gatctccatt ctccccaaag aaaggccctc acttcccact
cgtttattcc agcccggggg ctcagttttc ccacacctaa
ctgaaagccc gaagcctcta gaatgccacc cgcaccccga
gggtcaccaa cgctccctga aataacctgt tgcatgagag
cagaggggag atagagagag cttaattata ggtacccgcg
tgcagctaaa aggagggcca gagatagtag cgagggggac
gaggagccac gggccacctg tgccgggacc ccgcgctgtg
gtactgcggt gcaggcggga gcagcttttc tgtctctcac
tgactcactc tctctctctc tccctctctc tctctctcat
tctctctctt ttctcctcct ctcctggaag ttttcgggtc
cgagggaagg aggaccctgc gaaagctgcg acgactatct
tcccctgggg ccatggactc ggacgccagc ctggtgtcca
gccgcccgtc gtcgccagag cccgatgacc tttttctgcc
ggcccggagt aagggcagca gcggcagcgc cttcactggg
ggcaccgtgt cctcgtccac cccgagtgac tgcccgccgg
agctgagcgc cgagctgcgc ggcgctatgg gctctgcggg
cgcgcatcct ggggacaagc taggaggcag tggcttcaag
tcatcctcgt ccagcacctc gtcgtctacg tcgtcggcgg
ctgcgtcgtc caccaagaag gacaagaagc aaatgacaga
gccggagctg cagcagctgc gtctcaagat caacagccgc
gagcgcaagc gcatgcacga cctcaacatc gccatggatg
gcctccgcga ggtcatgccg tacgcacacg gcccttcggt
gcgcaagctt tccaagatcg ccacgctgct gctggcgcgc
aactacatcc tcatgctcac caactcgctg gaggagatga
agcgactggt gagcgagatc tacgggggcc accacgctgg
cttccacccg tcggcctgcg gcggcctggc gcactccgcg
cccctgcccg ccgccaccgc gcacccggca gcagcagcgc
acgccgcaca tcaccccgcg gtgcaccacc ccatcctgcc
gcccgccgcc gcagcggctg ctgccgccgc tgcagccgcg
gctgtgtcca gcgcctctct gcccggatcc gggctgccgt
cggtcggctc catccgtcca ccgcacggcc tactcaagtc
tccgtctgct gccgcggccg ccccgctggg gggcgggggc
ggcggcagtg gggcgagcgg gggcttccag cactggggcg
gcatgccctg cccctgcagc atgtgccagg tgccgccgcc
gcaccaccac gtgtcggcta tgggcgccgg cagcctgccg
cgcctcacct ccgacgccaa gtgagccgac tggcgccggc
gcgttctggc gacaggggag ccaggggccg cggggaagcg
aggactggcc tgcgctgggc tcgggagctc tgtcgcgagg
aggggcgcag gaccatggac tgggggtggg gcatggtggg
gattccagca tctgcgaacc caagcaatgg gggcgcccac
agagcagtgg ggagtgaggg gatgttctct ccgggacctg
atcgagcgct gtctggcttt aacctgagct ggtccagtag
acatcgtttt atgaaaaggt accgctgtgt gcattcctca
ctagaactca tccgaccccc gacccccacc tccgggaaaa
gattctaaaa acttctttcc ctgagagcgt ggcctgactt
gcagactcgg cttgggcagc acttcggggg gggagggggt
gttatgggag ggggacacat tggggccttg ctcctcttcc
tcctttcttg gcgggtggga gactccgggt agccgcactg
cagaagcaac agcccgaccg cgccctccag ggtcgtccct
ggcccaaggc caggggccac aagttagttg gaagccggcg
ttcggtatca gaagcgctga tggtcatatc caatctcaat
atctgggtca atccacaccc tcttagaact gtggccgttc
ctccctgtct ctcgttgatt tgggagaata tggttttcta
ataaatctgt ggatgttcct tcttcaacag tatgagcaag
tttatagaca ttcagagtag aaccacttgt ggattggaat
aacccaaaac tgccgatttc aggggcgggt gcattgtagt
tattatttta aaatagaaac taccccaccg actcatcttt
ccttctctaa gcacaaagtg atttggttat tttggtacct
gagaacgtaa cagaattaaa aggcagttgc tgtggaaaca
gtttgggtta tttgggggtt ctgttggctt tttaaaattt
tcttttttgg atgtgtaaat ttatcaatga tgaggtaagt
gcgcaatgct aagctgtttg ctcacgtgac tgccagcccc
atcggagtct aagccggctt tcctctattt tggtttattt
ttgccacgtt taacacaaat ggtaaactcc tccacgtgct
tcctgcgttc cgtgcaagcc gcctcggcgc tgcctgcgtt
gcaaactggg ctttgtagcg tctgccgtgt aacacccttc
ctctgatcgc accgcccctc gcagagagtg tatcatctgt
tttatttttg taaaaacaaa gtgctaaata atatttatta
cttgtttggt tgcaaaaacg gaataaatga ctgagtgttg
agattttaaa taaaatttaa agtaaagtcg ggggatttcc
atccgtgtgc caccccgaaa aggggttcag gacgcgatac
cttgggaccg gatttgggga tcgttccccc agtttggcac
tagagacaca catgcattat ctttcaaaca tgttccgggc
aaatcctccg ggtctttttc acaacttgct tgtccttatt
tttattttct gacgcctaac ccggaactgc ctttctcttc
agttgagtat tgagctcctt tataagcaga catttccttc
ccggagcatc ggactttggg acttgcaggg tgagggctgc
gcctttggct gggggtctgg gctctcagga gtcctctact
gctcgatttt tagattttta tttcctttct gctcagaggc
ggtctcccgt caccaccttc cccctgcggg tttccttggc
ttcagctgcg gacctggatt ctgcggagcc gtagcgttcc
cagcaaagcg cttggggagt gcttggtgca gaatctacta
acccttccat tccttttcag ccatctccac taccctcccc
cagcggccac ccccgccttg agctgcaaag gatcaggtgc
tccgcacctc tggaggagca ctggcagcgc tttggcctct
gtgctctttc ctggggtcac ctctgtctcc tcttggccat
tgggttctca caatccaaac ccgcgatgca aatttaggat
gtggctgtga agagagattc tgggtggaaa taaaaatact
ttggccttcc tggtcaagga ccagggcaga tcctgttgta
gtctccgtgc cccagggctg gcctgagaat gagcccctga
aaagacagcg ggtacgggca ccgtaagaac atcccctggt
ccagggtcct ctctctgaca atatttttgg tggccactgg
ccaccctgga actgggggtg cagaagattt ccccagtcag
aaccccattt cttgagtcgc atagctgagc ctggctcaca
caggcaggca ccctttgctt agacttaaag actgctccgt
cccctagcaa gggacaggca cttcctgctc ctccagcagg
gaatgtcgga ctgctggcca gaacagcagt ggcccaggga
ttgggtgctg gaggcctagt ttttcaccga tgggcctggc
tttttgcaaa ggctgggagg gatttggaga ggctgagcag
ctgggggctg aagacgggtg gaaagcctcc tgcccccacc
accccaacag cgccatgtga atccaagaag aaggaagggc
agggtgtagt cgtttttatt ctgaaatccc atttgaaatg
aaacttgaaa agaattcaaa actgggtcca gctgcagcca
cagacacact cagagggact ccaggaggct ggaacgtaga
ccagtgggcg ctgagaacct ggccggtggg ggtaggggtc
ttgattgcag ttttggctct tccacaccca ctgccaggca
ggtgtactgg tgcaggctct gagtgtgctt ggtgtctgca
tagaaggacg gttgttgaaa ggcaataaat caagtctttc
cctccacccc tgcacccaag ctttcagtag caaccagcca
ccagccaggc caggcaagac cagggcctct gaagaaggag
gggctgtgtc cagccaggct ttgggccctc ctccatgcca
gccgcctaaa ctgtgcaccc agctggaggc cttgaccacg
gtgggtgaga ctggagcagc tctggacgtg gaggaggaag
acactggcac acagtgcaca tcccctagaa caggtggcta
ctcgccgagg gtggccctgg actggtgggg gccaaggtag
aggactcagc cagtggctgg gctttgatgt agggcaggag
aagactgtgt gcaaccactt tgactttggt gggctcttca
ttggcagtgg gctcctcacc aagtagggaa gggaaagagg
taactgtttc cgggatctgc tgcagtcttc cctgccacac
tgcagtcccc tctggggagc at
NOR1
a. Primers
NOR1-F: TGAAGACGGGAGCTAATTGGTCTG
NOR1-R: TTCTGCCTGGGCTTTCCTCTGTTA
b. Amplicon
c. CpG island: Position: chr1: 36915797-
36916324; Band: 1p34.3; Genomic Size: 528
CGATGATGAGAGGGCCGGGCTGCTGGCTGCGGGTCTGGCTGAGCG
GGCCGGGGGCCTCTCACCTTTGCGGGCCTTGTCTCCCGGGATGTTC
TGGGCCCGCAGCCGTTGGTCGAGGATGTAAAGCATCTCCCCGCCCA
AGTTCAAGAAGAGCAGCGGTAGCGTCCGCACCGACATGGTGCTGG
AAACGAGCTGGACTGGTGAAGAGCCCCGGGGTTCGGTAGCCAGTG
GCCTGAAGGCCAGGCCGCAGCGTCCCAATAGTCCGGTTGCTGGGG
CAACGCCGTGACGGGAAGAGCGAGCCAATCAGAAGGCGGTTTGGT
GGGAGGTGCCCTGAAGACGGGAGCTAATTGGTCTGGGTGGTGGAC
CGTCCCGGGGGGATTGGTCCGAGCCAGAGGCCGGCGCGGCGTTGG
GCGCGGCTGGGGAGCTGTGCTTCTGAGAGTAGGTTTCCCTCGAAAG
GGCGAGGGCCGGGCCAGGGCTGGGGGTGGTCTCGACACAGCCAGC
CCGGCGCTTGGGACCCCGGCCGCTGGCGCG
SOCS1
a. Primers
SOCS1-F: AACACGGCATCCCAGTTAATGCTG
SOCS1-R: TTTCGCCCTTAGCGTGAAGATGG
b. Amplicon
c. CpG island: Position: chr16: 11348542-
11350803; Band: 16p13.13; Genomic Size: 2262
CGGCCTCGTCTCCAGCCGAGGGCGGGAGGCGCCTCGCCCCTACAC
CCATCCGCTCCCTCCAACCCAGGCCGGGGAGGGTACCCACATGGTT
CCAGGCAAGTAATAACAAAATAACACGGCATCCCAGTTAATGCTG
CGTGCACGGCGGGCGCTGCCGGTCAAATCTGGAAGGGGAAGGAGC
TCAGGTAGTCGCGGAGGACGGGGTTGAGGGGGATGCGAGCCAGGT
TCTCGCGGCCCACGGTGGCCACGATGCGCTGGCGGCACAGCTCCTG
CAGCGGCCGCACGCGGCGCTGGCGCAGCGGGGCCCCCAGCATGCG
GCGCGGCGCCGCCACGTAGTGCTCCAGCAGCTCGAAGAGGCAGTC
GAAGCTCTCGCGGCTGCCATCCAGGTGAAAGCGGCCGGCCTGAAA
GTGCACGCGGATGCTCGTGGGTCCCGAGGCCATCTTCACGCTAAGG
GCGAAAAAGCAGTTCCGCTGGCGGCTGTCGCGCACCAGGAAGGTG
CCCACGGGCTCGGCGCGCAGCCGCTCGTGCGCCCCGTGCACGCTCA
GGGGCCCCCAGTAGAATCCGCAGGCGTCCAGGAGCGCGCTGGCGC
GCGTGATGCGCCGGTAATCGGCGTGCGAACGGAATGTGCGGAAGT
GCGTGTCGCCGGGGGCCGGGGCCGGGACCGCGGGGCACGGCCGCG
GGCGCGCGGGGGCCGCGGGCGAGGAGGAGGAAGAGGAGGAAGGT
TCTGGCCGCCGTCGGGGCTCTGCTGCTGTGGAGACTGCATTGTCGG
CTGCCACCTGGTTGTGTGCTACCATCCTACAGAAGGGGCCAGCCGG
AGGGGTGGGCCATAGCGTCCGGGGGTGCGCTGCGGGAGAGACAAA
GAGGTGAGCTGGGGCGCTGCGGGGCCGGGCAGGTGTGCGCCGGCC
GGACAACTCCGGAGGGCGGCGCTCCCGGCGGACCCGGCCCTAGGG
GGCGAGCACGGAGCACCAAGTCCGCGCGGATCCGTTCAGCCTCAG
TGGACACAGCTAGAAAATGGGCTCTGTACTCCGCGGAGCTCTTCCC
GGCGGGTGGGGGCTCGGTGGAGGCGGAGTCCGGCCTCCGGGCAGC
ACCGAGAGGGGGGCGTGGAGAGCAGCCGGTTCTGGCTCCAGCCGT
CCGGCCCCGGCTCGCCGCCCCGCGCCCGCCGCCTGCTGGCCAGGCT
GGGATCCGCGCCTGGTCTGGGCGATTTGGGCTAGGGCCGGAGAAA
GGCTGTGCTGCGGGAGCCCCGCGCGCGGGGGGCGGCCTGGGTGGG
GCCGGCGAGGGTCAGGGGCATCGCGGCCGCGACCCCATTCTGCAG
CCCCCGAGGCTCGCCCGACTCCTGGCTGCCCTGGACTCCCCTCCCT
CCTCCCTCCCGCCTCCTCGCCCAGGGCCCGGCTCACCTGGCGGCGG
GGCGCGGGACGCCGCGGGCGGGACGGCGGGGGGCTCCGGGGCGC
TCCGGGGCGGCTCTCGCGCATGCTCCGGGGCCAGGAGCCGTGCAG
CTGCCACGGCCGCAGCTCGCTCTGTTCGGCGCCCGCCCCTGCGCCA
GTCTTTTAAACCGGCTCGGAGGCGGGGCTGGCGACGGCGGGAGGC
CCCGCCCCCTGCCGGCCCCGCCCCCAGCTCCACTTTTGGTTTCTCTT
TCCGCGGTGGCGTCCGGCGAGGACCGCTTCGGCCCTGTTTCCCTCT
CTTCTGGACCCTCCCGCGGGGCCCTCTGCCCGCCTGTTCGCACCTG
CCCCAGCACCCGCCTCTCGAGGGGCTCTGGCCCCGACCCTGCGCCT
TCCGGCCACTTCTCGGACCCCTCCTTCGGACTTGGCGACCCCGATT
TTGCCCCGCTACCTCGGGTTCCACTTTCTGCCGCCAGGCCCTCTTGG
GACGCGCCCTGACACACCCTCCTCCGCCCCAGCTGTCTCCACACCC
GCCGGGGGCAGAGCCCTGTCCTCTCCTCCCCTGCAGCCAGATCCCC
CTAGGAGGCCACAGAAGGTGTCCCCAACCCTGAGCCTGACCCCAC
CCGTAGACCCCCTCCTAGCCCCTGCTCCACCCGCCGTCGACGCCCT
CAGTCGCCCGCCCTGCTGTCCCGAAGCCCCGGCCGGCCGCGGTCTC
TGGTCTTGGCTCGGGCTTCCCGGGAAGCGGCGGCCTGACCACAGG
CTTCAGAGGAACCCCTGGCGGCGCGGGCGCCTCCACCCCGGCCCA
GTTCCTCGGAAACTGGGCGGGGCCGGGCAAGGTCCCTGGTGGCCT
CGACTGCCCTCCCTGCGCTCCCACTACCCGGCTGCG
RECK
a. Primers
RECK-F: TGAGTAACCTCCAGAGCAACGGTT
RECK-R: TTTCTGACAAGCAGCAGAGGCAAG
b. Amplicon
c. CpG island: Position: chr9: 36036799-
36037564; Band: 9p13.3; Genomic Size: 766
CGGGGCACGTTCCCGCCCCCGGGAGGTTTTGGAAACACTGTGAGG
CAGGGGGCGGGGCTTGAGCGGGCCGCAGCCAGTCACCAAAGGGCC
GGGCGCTGGGGGCGGGGCCTCGCGCGAGCGGCGGCGGTAGCGGCG
GCAGCGGCTGCGGCCAAGCTGGGTCCGAGCATCCCGCGGCTCTGG
AGCCGCCCGGCCCGGACATGGCGACCGTCCGGGCCTCTCTGCGAG
GTGCGCTGCTCCTTCTGCTGGCCGTGGCGGGGGTCGCGGAGGTGGC
AGGGGGCCTGGCTCCGGGCAGTGCGGGTGAGTAACCTCCAGAGCA
ACGGTTCGAAGCTGTCGGGAGCGGCCGCCACAGCGCTCCAAGATG
GCGCGGGGCAGGGGGCGGGGGTGCGCGCGACCCCCAGACCCTGCC
CACGTCCGGCGACCCCGGGACCCCAGGTCTCAGCGCTCCAGAGGC
TGGTGCCGAGGCGGGGCGAGTGAGGAACTCTCTCCGCCCCAAGAT
CTTCTGGGCGGTGACTCGGGTTTGAGGCCTTGGTCTGTCACCCACC
GACACGGGCCCCCTCTTCGGCACTGACCCCTTCGCTTGCCTCTGCT
GCTTGTCAGAAAAGGGTGCGATGCCCCCGCCCAGGATCGTCGCGA
GGTTTAGATGGGATTTCGGATACGCAGCCGCCCTACCGCGGCCCTA
GTTAGTTATTGTTACTTGTTACTTGACCCGCACTTGGTTCATAACGA
CCTTGGTGGCGGTGAGCACTGACGGTCCCCACAGCCCGCG
MAFB
a. Primers
MAFB-F: TCGTGCGTTCCTGTTTCTGGAGAT
MAFB-R: CGCACTTTATGCCTGTTTGAGCCT
b. Amplicon
c. CpG island: Position: chr20: 39316551-
39319987; Band: 20q12; Genomic Size: 3437
TTGACCTTGTAGGCGTCTCTCTCGCGGGCCAGCCGGGACACCTCCT
GCTTAAGCTGCTCCACCTGCTGAATGAGCTGCGTCTTCTCATTCTCC
AGGTGGTGCTTCTGCTGGACGCGTTTATACCTGCAAGACTGGGCGT
AGCCCCGGTTCTTCAGGGTCCGCCGCTTCTGCTTCAGGCGGATCAC
CTCGTCCTTGGTGAAGCCCCGCAGGTGGCGGTTCAGCTCGCGCACG
GACATGGACACGAGCTGGTCGTCGGAGAAGCGGTCCTCCACGCTG
CCGTTGCCGCCCGCCGCCGTCGCCGAGGCCGTCGCGTGCGGCCCGG
GCCCGGGGTGGCTAGTGGGCAGCTGTTGCGCCGGGCTAGCGGCGC
TGGACGGCGGCGGCGACGCTTGGTGATGATGGTGATGGTGCGGGT
GAGCGTGCGGGCCCAGCTCGTCGTGGGCCACGCCGGCGCCCGGGT
ACGCGTGGTGCGGGTGAGGGTGGTGGTGATGGTGGTGGTGGTGAG
CGCCGCGAAAGCTGTCGAAGCTTTGCAGCGGCTGTGGCACTGGGT
GCGAGCCGATGAGCGCTTCCACCGCGTCCTCGGGCGTCAGGTTGA
GCGCCTCGGGGTTCATCTGCTGGTAGTTGCTCGCCATCCAGTACAG
ATCCTCGAGGTGTGTCTTCTGTTCGGTCGGGCTGAAGCTGGGCGAC
GAGGGCACGGAGCTACACGGAGTGCTGAGCGGTGTGGAGGACACC
GAGCCGGCTGGCTGCAGGCGTGTGCAGGGCCTGCCCGGACGCTCC
GCGCGCCCCAGTGGCTCCTTCTTCACGTCGAACTTGAGCAGGTCGA
AGTCGTTGACATACTCCATGGCCAGCGGGCTGGTGGGCAGCTCTGG
CCCCATGCTCAGCTCCGCGGCCATCGCTGAAGCGAGGCGCAGCCG
CCGCTGCCGCCCGGGAAACTTTGCGGCCGGCCGGAGCGCGCCGAG
CCAAGCGCGGGGGGGAAGAGCGGAGAAGAGCTGGGGAGGCGGGG
AGCGAGGGCGCAGCGGGCCGGGGCCGCCGGCCAAGCCTTTGTCTG
GGGACGCGGCGGCGCGCCGGAGAGTCCCGAGGCTGCCTGCACCGC
CCCAGAGCTCTGGGCTGTGCCCGCGCAGGGACCGGGCCGGGTAGA
GTCGGGCGGGGTGGAGAGGCAAGCGGAGCGCGCGGTGGGGCTGA
GGGGAGGCGTGGGGCGAGTGCCCGTTGCTCGCTCTCTAGCTCTCTT
GCTCTTACGCTCTCTCGCTCGCAGCCGCTCGCAGCTCGGCGGTGCA
GCTGTGCTGGATCCGGCGGCGCCGCAGCCTTTTATCGCCTCCTGAT
GTCACTGGGGTGCGGGGGCCCGGGCGGCCCGGTGCGCGGGCCAAT
AGCTGCACGGCCTCCGCGGCCCAGCGGCGCAGGGCGGGGCGCGCC
TGACAGCTCCCCCGCCCCCCGCGTCAGCTGACTGGCGGCCCGAGCG
GCCCCGGAGCGGCGGAGGCCTGGCGGAGCGCTGGAGCGGAGTGG
GACGGCCAGCCTGGGCCCACCCCCGTACCCTGCAGGTCCCGGCCC
ACGCACGCTCGCCTGGAGTGCGCGCCCCACCTCTAGGCCAAATCAC
CGCTTTCCCCTCCTCGCGCACTCTCCTCCCTCAGTTCCCTTTGCACC
CCACCCCCATCCCGTGTCACCCCCAAGGAGGCTCAGAATGAGCGC
CGGGACAACGCCTCCTGGGCCCTTTGTTCCCAAGCGGCCCCCGCCC
AGTGGGCGACGCTCTGTGTGTCCTCGCGGCTTCTGGCCGTGTGTGT
CGTGCGTTCCTGTTTCTGGAGATCTGCGCGTATTTGTATGTTGGGGA
GGGCGGGCTCGAGGCTCCGAGAGTTGTGTTCAGACCCAACTCTTAA
CCTCAGGGGACCTTTCTCAGGCCAAGCGAGGGCCCCTCCTGGCGG
GTGCAGTCGCAGAGCCCTGAGGTTCGACTCCACTGGCCCCGCCGCT
CCCCGCGTTCACCCCACCGCACAATGTTCACAGTGAAGGCGACGG
GAAAAGCAGCAGCCCAAAGGCTCTGAATTCCTCTTCCCCGCCACAC
GCACGGAATCCTGAGCCCCCGGAGCCTCGGGGCCGAGGCCGGCCC
GGGACGGTGCTCCGAGTAGCTCTCCACTGCTGGGGAGCCGGCCCT
GTTTTTGTTTGAACGTTTTGTAACGATTAAGCAGATCCCGGCGTCA
GCCCGCCGCGGAGAGGCTCAAACAGGCATAAAGTGCGACCCCAAG
TGGCCACTGTGCGCAAAGGCGCCGCGACCGCCCGGCCCACGGCCG
GAAGGCTTGGACGGCGCCTCGTACCCAGCCAGGTCTCCCCTACCTG
GCCCAACCCAAGCCAGCCCAGAACGCATACTATGTGTGCACCAGA
GCCCAGGACAGGTTCCCCTCGAGCGATGTACAGGTCCTCGGGTCCC
GTCTTCGTACTCAGCCGCGAGCCTCGAGCCGCGAGCTCCGCTCTGG
TCGCCCCGTTGAAATTCCGTGCCCCAGCGTTCGGGGGTGCCCGTCG
GCTGCTCCCTGGGCCGGAAGGTCCTGGGCGGAGGAAGGCCGGTAG
CCAAAAGTGGAAGCGCCACAGTGAAGCGGCCCAGGGCCACCGGGT
GAGAAACCTCCCCGGAGGGCAGACGGGGAGACCGAAGCACACCG
CACTAGGCATCCAGACTGGGCTTGGGAGCCGCGCACCCTCCCTACC
CAGATCCAGGATGGCTAGAATTAACGGGTTCTTTCTGAGACCTCGG
CTCAGGCGCCGAAACCGGATAGATCGCGAATTCGCTGGACCCGGA
GACCCGACCCGCCTCCCGCGTCACCTTCTTCTTTCTAGCTTTGGGCG
CGCGCAGCGAAAGGCAGGAGAGGCGCGCACTGGGTGAGTGAGTCC
CGGCCGCTGTCTGCGCTGGACCAGCCCGACTGACCTCGCGCGTAGG
GGTCGCGTGAGCCACACCGGTGCAGACGCGCCTAGATTATTTTTAA
ATGTTAGAAGGTAAAATATTTGCCTCCAATTAATCTGAAAACTCTC
TATTCTCTTGCGCCCTCGGAGAGGCTGGGGTACGGCGTGGTATTGG
GCCGCCTATTTTTAATAAAATGAGTGTATTTTAACTAAAACTTAAC
TCAATCTTGTGGGGTGGCAAATTAAATGCTGGAAGAGCGCGTCTAC
AACCCTCTTCGAGAAGCGTGCTCTCCGCAGAAATGAGTCGGCCGCC
TGGAGAGAGAGCCTGGGCGGTGCCGCTGCGCAGCCCCTGCCAGTA
GCTGGGGGTTGGGGACTCGCACCTTGTAAATGTCCTCGTCTTGTTT
GAACGCAGTGAGAGCACACTCGTTTCCAGATCACTCGGGACCGGG
TGTCTCGGATCTGTGCAGACTATGTATGGCTCCGGCCTCAGGCGGC
CAGGGCGGGACAAGCACG
p15
a. Primers
p15AF: ACATCGGCGATCTAGGTTCC
P15AR: TTTTCCCAGAAGCAATCCAG
b. Amplicon
c. CpG island: Position: chr5: 32585604-
32586365; Band: 5p13.3; Genomic Size: 762
CGCCCCATCACGTGACCGCAGCCCCAGCGCGGCGGGGCCGGCGTC
TCCTGGCTGCCGTCACTTCCGGTTCTCTGTCAGTCGCGAGCGAACG
ACCAAGAGGGTGTTCGACTGCTAGAGCCGAGCGAAGCGTGAGTGC
GCGGGACCCCCTACCCCTACTCCTCGGGGCCCCCACCCTCCCAGCC
GGGCCGTGAGCTGCCTTCGGCCCTCCACTCCTCTCGCCGGCAATGG
CCGCGGGAAATGGCGGCTCTGCCTTACCTCCCCCTTCCCCTCGGCG
TCCCCGGCCCCCTTCTCCGTTTCTGACTCCACGCCTGACGCGCTGTG
GGCCCTTCCGCGGTAGACTCCTGTCCCCGGGGAGCCGAGTCGAGG
CGGCGGGCGCTGCGGCCCGGGGCGGTAGATTGAGGGCGGCCGGGG
AGTGAGGAGTCGCGGGGAGAGAGTCGCGGCGTCCCCGGGACAATG
CGGCGGCGGCCTGCCTAGGTGGGGCGCGTGCGGTTACCTACTCTTC
CCCCGCCCCTCGCCCTGAGCGGGGCGCTCTGGAGACTGGGAGAGC
GGATGCGGGCGGGAGGGGGCCGGGGGAAGAACGGCTGATGTGCA
GGGGGAGGGAACGCTTCGAGAGAAGAAAATGGCGCTTGGTGCAAA
TCCCGCCCCTTCCCACGCCGTCTTCTCCGCACTTCGCCGCCTCCCAC
GCCCCCTCCGACCAACCTGTCTCCCCTCGCCCGAGCGGCTGCTAGC
CACGGGGTTCTAGCGGCTTGCTGGGGCCGCGCG
HOXD11
a. Primers
HOXD11-G1F: GACATTTCTCTTCATGGCGTC
HOXD11-G1R: CAGACGGGGCCACATAGTAG
Amplicon
b. CpG island (Position: chr2: 176971707-
176972305; Genomic Size: 599)
CGGGCGGTGGCAGATGCGCCCAGCGGTGACAGCGGCCAGCGGCGC
GCAGGTGACCGGCCTGAGGCGCAGCCTGGTCAGGGAGCGCCCGGG
GAGAGCTGGCGGCAGAGGGCAGCCGATCCGCCCCCAGCGCGCGCG
TCTCGGCGCCAGGAGCCGTCCCGGGGCGTGTTGGCGAGCGTTGAT
ATAGATATAAGGACATTTCTCTTCATGGCGTCACGTGACATAATTA
CCACCAGAATCAATCAAGATGAATTGCACGTCAGCGCCCGGTGGG
GATTTTTGCTTAGTTGATCCTGGCCCAAGCCTCTTGTGCAATCGATG
GCTCAGGTTGGCTGCGCGGGGAGCGGCCAGAGGCTCGCTGGCGCG
CACGCCGCGGAGTCATGAACGACTTTGACGAGTGCGGCCAGAGCG
CAGCCAGCATGTACCTGCCGGGCTGCGCCTACTATGTGGCCCCGTC
TGACTTCGCTAGCAAGCCTTCGTTCCTTTCCCAACCGTCGTCCTGCC
AGATGACTTTCCCCTACTCTTCCAACCTGGCTCCGCACGTCCAGCC
CGTGCGCGAAGTGGCCTTCCGCGACTACGGCCTGGAGCGCGCCAA
GTGGCCG
HOXA11
a. Primers
HOXA11F: AAAACTGGTCGAAAGCCTGTG
HOXA11R: CCTTCAGAGAGTACGCCATTGA
b. Amplicon
c. CpG island: Position: chr7: 27219310-
27219750, Genomic Size: 441
CGCGCGGCGACGCTCGCGAGGCCTAGCGAATGCGCGTTGCTTTAA
ATTACCATACCAATCACTTCTTGAGGGTGAGTCCCCTTTTTCTGTTA
TGAAGGGGAGCGGGACAAGTGAAATAATGTACCGTGCTGCTCTTA
GTATCAGAAGCGAACAAAGGCCAAGAATCATGCTGGGGTTCCCGG
CTCCCCGGCGGCTTTGACATTGATCGGAAGTGCGCCATCTCGTGGC
GGCTGCGCGCCTAGGTTGGGCCGGAGTTCCAGCCCCGAGCCGAGA
GACGGAAACCAGCTCCGGGCAGAGAGAGAAGGAGAGAGGAGAGG
ATGTGCCCAGCCCGCTGCTATTGAGATCTCATTTTTACATCTAAGA
AATCGCTGCAAAACCCCAGCCGGGTTTATAGCGGCGCATTCCAAAT
ATGCAAATTGGCCGGCCCCGGACGGGTTTACG
HOXA6
a. Primers
HOXA6F: GGACCGAGTTGGACTGTTGG
HOXA6R: GATTTGCTGCTGTCGCTTTT
Amplicon
b.CpG island Position: chr7: 27182614-
27185562; Genomic Size: 2949
CGAGAGCCGCGTCCCCGCGGTCGCGTGGATTTAGAAAAAGGCTGG
CTTTACCATGACTTATGTGCAGCTTGCGCATCCAGGGGTAGATCTG
GGGTTGGGCGGGCGGCGCCGGGCTCGGCTCGCTCTGCGCACTCGC
CTGCTCGCTGCTGGCAGGGGCGTCCTCCTCGGCTCCGGACGCCGTG
CCAACCCCCTCTCTGCTGCTGATGTGGGTGCTGCCGGCGTCGGCCG
AGGCGCCGCTGGAGTTGCTTAGGGAGTTTTTCCCGCCGTGGTGGCT
GTCGCTGCCGGGCGAGGGGGCCACGGCGGAGCAGGGCAGCGGATC
GGGCTGAGGAGAGTGCGTGGACGTGGCCGGCTGGCTGTACCTGGG
CTCGGCGGGCGCCGCGCTGGCGCTGGCAGCGTAGCTGCGGGCGCG
CTCTCCGGAGCCAAAGTGGCCGGAGCCCGAGCGGCCGACGCTGAG
ATCCATGCCATTGTAGCCGTAGCCGTACCTGCCGGAGTGCATGCTC
GCCGAGTCCCTGAATTGCTCGCTCACGGAACTATGATCTCCATAAT
TATGCAACTGGTAGTCCGGGCCATTTGGATAGCGACCGCAAAATG
AGTTTACAAAATAAGAGCTCATTTGTTTTTTGATATGTGTGCTTGAT
TTGTGGCTCGCGGTCGTTTGTGCGTCTATAGCACCCTTGCACAATTT
ATGATGAATTATGGAAATGACTGGGACATGTACTTGGTTCCCTCCT
ACGTAGGCACCCAAATATGGGGTACGACTTCGAATCACGTGCTTTT
GTTGTCCAGTCGTAAATCCTGCCTGATGACCTCTAGAGGTAAACTC
GTGCACTAATAGGGGAGTTGGGTGGAGGCGAGGGGGGTGGCGCGC
GCGCCCCGGGCGCGTGCCCGCCGCCAGTTGCCGCCGTTCAGCCGG
ACTCGAGCGCCACCCGCTGGAGGCAGGGCTCATCGCCCAGCTTCC
GACCGGGGGCTGCAAGGGCCGGGGTCGAATTGAGGTTACAGCCCA
TTATGGCAAAATTATTGCATTTCCCTCGCAGTTCCATTAGGATGTAC
CAATTGTTAGGCCGTCAGCTGCCGATCGCGCGCCCGGCGAGGATG
CAGAGGATTGGGGGGAGGTGGTGACTTGCATTTTATTTACAACAAC
TTTATTTCCCCCGTTTTGCAGCCCCTCTTATTTTTGTGTCGAGGTTG
GGGTCGGTACTGACCGTCCTGCCAGCAGCTCTGAATTTTGAAAATA
CAGATATCACCTTCGGGGAAGGGGGAAAGCCATTTAGCCAATTGG
AGAAATAAATCCTGCCCGCAGCAGCAGCAGCTACAATTACGGCTC
TGTTTTTGCGAGCGCATGAGGGACAGTGTCCCTGCCGCTCTTAAAT
GACAGGCGTCTATTAAAGATAGCTTTTGTGTAGTGTTTCTCCAAGG
CGAGGTCAAATTCCATACACTTTTATAACCGTAGTCGATTTTTCTTT
CGTGTGAATATGGTTTTCGTGTCATTAGTTTGCGATTTGATTTGCTT
ACGTATCCAGCCTGGAAAATCTTCATCACAGGGTCCGGTTCCTCGA
GCCAGCCGGGCCCCAAGTCGGAGGGTTCTCCTTGAACCCAGCGAG
TGGGCCCAGGCTCCCTGCAGCCACAGAGGCTGCCTGGGGTCTGGG
GATCCGTGGGGCGGGTTACTGGGGTCTTGCTTAGACCTCCAGGAGT
AAAATGAGGGCGATAATGGAAGCATTCCTTGGCAGTGCCTAGTAT
CTCTGTAGTTATTTTCCACGGCTCCGAAAGACTCAAGTAAATCACA
AATATAGCTGAGAGGCAAGTGGAGTCTCCCCGCTGGAGGCCCGGC
GTTGCAGGCGCCCCTGGCACGTCTGGAAGCCAGGACTCTGGCGGC
TCCCATGGCCCTGGGCCCCTCGTTGGGTCCTGAACGCTGCTGTGGC
GGCGACGCGGGCGCTATCGGAGGCTGGGAGCGGGAATCCGGAGCC
GGGAGCCTACCCCGGGCTGTAATGTTCCACCCGCGCCCAGGTTAAC
TCGCCTCGGCTGAGGCTGCTTCTCTTCCACTGACGGTTGCACACGC
GGGACCGAGAGACTGGGCTCTGTTGGGGCCCCCTTTGTTCCTCGAG
CTTCCTTCCTGTTCTGGGAGGCGGCTTGGGAGGCCGCGACAAGGCC
GGGCTCCAGCTCTTAGACCCCCTCTTTCCACTGGCCAGAGATGATT
TGATGATGCCCTTCGGGACTTACTGGCGAGGGACTTAGGCAGAGA
CGCCCAGACACGAAACGGGGCTCGGCCCAGGGCTCTTTCCTCCCCA
GCAGCCCCGCGTCCCGAGGTCGGGGAGCTCAGAGACACTAGCACA
GGAGCCCCAGACGCATTCAGGGCGCACCCCAGAACTCCGGAGCCG
GTTTGGGCATCCTTGTGGAGCGGGACTGGGTGTGTGCAGTGCGCCC
CGCTCCACCGCTGGTATTGGCTGTGTGTGAGGTTTTGTTTTGTTTTG
TTTTGTTTTGTTTTGTTTTGTTTTGTTTTGTTTTGTAAGAAATAAATG
CACAGACGCTTGCAAAGCTCCGGGCTCCCCTGAAGCTGCGGAAGC
CCCCAGATGGGAGCAGGCGGGGAGAAAAGTTGGGGAACAGGCGA
GGGCAAGGGGGCAAAGCCGAAGGAGGTTGCAGCGCTGGCCTGGTC
CCTGCCCAGGCATCTACTCGCCCGCCTTTGCCTCTGAGTCCTCCCCG
CTGGGCTGCGTGGAATTGATGAGCTTGTTTTCCTTTTTCCACTTCAT
GCGGCGGTTCTGGAACCAGATCTTGATCTGGCGCTCGGTGAGGCAG
AGCGCGTTGGCGATCTCGATGCGGCGGCGCCGTGTCAGGTAGCGG
TTGAAGTGGAACTCCTTCTCCAGCTCCAGTGTCTGGTAGCGCGTGT
AGGTCTGGCGGCCTCGGCGCCCATGGCTCCCATACACAGCACCTAC
GAGCAGAAACGGCCGGGCGCCG
HOXA7
a. Primers
HOXA7F: ACGCAAAGGGGCTCTGATAA
HOXA7R: AAAGCTGCCGGACAACAAAT
Amplicon
b. CpG island: Position: chr7: 27195602-
27196567; Genomic Size: 966
CGCAATGGCGCCTCCGCTCCAATTAAAACCAGAAAGGCTGCGCCG
GGAGTCACGGGGCTACCGGCTCGCAACAGCCTGGCTCCGCTCTTCC
GGCCCCGCGCCCCGCGCTCCGCGCTCCCCAGCGCTGCGCTCCCCGC
TCCCGGTCCCGCTCCGCCAGCCTGGCCCGCCTAGCGACTGCGCCTA
CCTGAAGACCGCATCCAGGGGTAGATGCGGAAATTGGCCTCAGCC
GCGCCATGCAGCGCGCCCTCGTCCGTCTTGTCGCAGGCGCCTTTGG
CGAGGTCACTGCAGAGCCCGGGGATGTTTTGGTCGTAGGAGGCGC
AGGGCAGGTTGCCGTAGGCGTCGGCGCCCAGGCCGTAGCCGGACG
CAAAGGGGCTCTGATAAAGGGGGCTGTTGACATTGTATAAGCCCG
GAACGGTCGAGGCGAAGGCGCCGGCGCCCGCCCCGTAGCCGCTTC
TCTGTGAGTTGGGAGCAAAGGAGCAAGAAGTCGGCTCGGCATTTT
GGAACAGAGAAGCCCCCGCCGTATATTTGCTAAAAAGCGCGTTCA
CATAATACGAAGAACTCATAATTTTGACCTGTGATTTGTTGTCCGG
CAGCTTTCAGTGTCGGTTTTACGAGGTAGAGTGATATATGATAACA
TTACACCCCCAGATTTACACCAAACCCCATTTTCTTTTGGACGGAG
CTCGCCGCAGCACGTGACCGCCCACATGACCGCCTCCGCCAATCTC
AGCAGTCCTCACAGGTGGTCTCGCTCCGCAGGGCCCGCAGCCGCCT
AGAATGGAAGGGCAAGAGGCTCAAATATGCGGCCAAAGAATCCGC
CCGCGCCCGGCGGGCCTGGCGCGTCCCGCGGAAAAAGACCTGGAG
GCTCCGCGGGAGCGCCCAGCTGGCGGCCAACCTCCGCACTGGGGT
CTGCGGACGCCAGGCGGCCCGGCCCCACGCAGCACCCCCCACCCC
GCCCCCCCGCCG
HOXD9
a. Primers
HOXD9-G1F: CTAATTGCGGCGCTTATGTT
HOXD-G1R: TGGCCTATAAGCGAGTCCAC
Amplicon
b. CpG island: Position: chr2: 176986425-
176988291; Genomic Size: 1867
CGGCCGAATTTTTTAGACATTTTGGGAGTCTCCTCCGAGGCCTTTA
AGTGCGAACCGCGCGAAGCGGCCCTGCCCGGGGAGACTCGCTGAG
GCAGGGCTGAGGCGGCGGGCGGGAGCAAGCTGCTCTAGCATTTGG
GTTCTGCCCTGTGGCGTGTTCTCTTCCAGGGCCTTTCCAGCATCATC
GGAGAAGACGAAGCACCCTGGCCGCCACTGTCCGTGCTGCGCCAA
CTCGCCCGGCCGCCCGCCCTTCCGAGGGCAGGCAGAAGCCCCTCTG
TGTCCTCCACCGCCGCGCCCCGGCTCGCCCCTCGGGCCGCGGCGTG
TGCCCAGCCTCACGTCGGGGTGTGTGTGGCCGCGCGGGCGTGTGTG
AGTGTGGCAGGGGGAGGGGGCCCTCCGATCTGCTCCATCCGTCCGT
TTTATTAGGGACACATTAATCTATAATCAAATACACCTCATAAAAT
TTTTATTGAAAGGCATAATATCATTACAGAGGTCTTCCACCTGTTTT
AAACAACACGACAAGCTGTGAGCAAGCGTGTGTGTGGGGATGTGT
GGGGAGGGGTGGGTGTGAGTAGGGAGAGAGGCGAGGGGAGAACA
GCTCCCCTCGGGCGCTAGGGGCCGCCCCGAGGGCCCGCCTGCCTCG
GGCGACACCGGCCTGGCGCCCCCGCGGCCGCTCCGTGTGCCCTGG
ACTCGCCGCCCGCGGCTCGGAAGCTGGAGAGTCAGCGACGGGGCC
CGACTGCGGGACCGAGGGCTGCAAGAAGAAGCGAACAAATAGTCC
CCAGCGCCTCCTCTGGATGCGGTCGCGTCTGTGGTCCTGGCAGCCG
CTGGGCGGGCCAGGCCAGGTCGGGCCGGGCCGAGCCGGGCACATG
GACCTGGGCCTGCGGGCTCTAATTGCGGCGCTTATGTTGATGATTT
TTTTTTTAATCACAGCAGCCCCCAGTTTAGCGGACTGATTTACTCCC
GGTATTGGTAAATATGATCACGTGGGCCGCGCGACCAATGGTGGA
GGCTGCAGCCTGCGAACTAGTCGGTGGCTCGGGCGCCGGCGGGGA
GCTGCTCGGCGGCGGACAGTGTAATGTTGGGTGGGAGTGCGGGAC
GCCTCAAAATGTCTTCCAGTGGCACCCTCAGCAACTACTACGTGGA
CTCGCTTATAGGCCATGAGGGCGACGAGGTGTTCGCGGCGCGCTTC
GGGCCGCCGGGGCCAGGCGCGCAGGGCCGGCCTGCAGGTGTGGCT
GATGGCCCGGCCGCCACCGCCGCCGAGTTCGCCTCGTGTAGTTTTG
CCCCCAGATCGGCCGTGTTCTCTGCCTCGTGGTCCGCGGTGCCCTC
CCAGCCCCCGGCAGCGGCGGCGATGAGCGGCCTCTACCACCCGTA
CGTTCCCCCGCCGCCCCTGGCCGCCTCTGCCTCCGAGCCCGGCCGC
TACGTGCGCTCCTGGATGGAGCCGCTGCCCGGCTTCCCGGGCGGTG
CGGGCGGTGGCGGTGGTGGTGGAGGCGGCGGTCCGGGCCGCGGTC
CCAGCCCTGGCCCCAGCGGCCCAGCCAACGGGCGCCACTACGGGA
TTAAGCCTGAAACCCGAGCGGCCCCGGCCCCCGCCACGGCCGCCT
CCACCACCTCCTCCTCCTCCACTTCCTTATCCTCCTCCTCCAAACGG
ACTGAGTGCTCCGTGGCCCGGGAGTCCCAGGGGAGCAGCGGCCCC
GAGTTCTCGTGCAACTCGTTCCTGCAGGAGAAGGCGGCAGCGGCG
ACGGGGGGAACCGGGCCTGGGGCAGGGATCGGGGCCGCGACTGG
GACGGGCGGCTCGTCGGAGCCCTCAGCTTGCAGCGACCACCCGAT
CCCAGGCTGTTCGCTGAAGGAGGAGGAGAAGCAGCATTCGCAGCC
G
HOXA9
a. Primers
HOXA9-G1F: AGCAGGAACGAGTCCACGTA
HOXA9-G1R: TGCAAAACATCGGACCATTA
Amplicon
b. CpG island: Position: chr7: 27203916-
27206462; Band: 7p15.2; Genomic Size: 2547
CGGAGCTGGGCAAGCCGTCAGGGCGCCCTAAGGCCGCTGATCACG
TCTGTGGCTTATTTGAATAATCTGTCATGGGGACCCTTGTGGCCCG
GGTCGCCCGCAGCCTCATCTTGGCAGGATTTACGCCGCCACTGGCC
GAAGGCAAGAAGTGGAAGGAATCGGCCGTCTCCCCCAGCGTCCCA
GCTCCGGCTGCCCTGGCTGCCGCCGCTCACGGACAATCTAGTTGTA
CAAAAGGCTCTCTGGGCTGCACTGCTTTCGAAGAACGGCCCAAAG
TATCTCGGTCCTGGGCCTGGGCAGCCAAGGAGAGGGGCGGCCAGT
CTTGGCTCGTCCCGAAGTGCCCGCCCCGCCCCCTCTCGCTGCAGCA
GCCGCCTCCTCTCCCGTAGCCCTGCGGGCCGCTCTTCACTGCTCTCC
AGACTTGGGGCCCTATCTGAGGCGTCCCAAACACCAACTTCTGGCT
CCTGGCCCCAACTCGAGAGGCTTCCAGCGAGGACGAAGGCAGGCT
CGAGAGAAACCTGGCGGGCCAGCAGATCCGGGAGGCCGGCGTGG
AGGCGGCGGCGGATTTGAAGGGAGGAGACACTTACTGGGATCGAT
GGGGGGCTTGTCTCCGCCGCTCTCATTCTCAGCATTGTTTTCAGAG
AAGGCGCCTTCGCTGGGTTGTTTTTCTCTATCAACTGGAGGAGAAC
CACAAGCATAGTCAGTCAGGGACAAAGTGTGAGTGTCAAGCGTGG
GACAGTCACCCCTTCTGGCCGACAGCGGTTCAGGTTTAATGCCATA
AGGCCGGCTGGAGGGCAAGCCCGCGAAGGAGAGCGCACCGGGCG
TGGGCTCCAGCCAGGAGCGCATGTACCTGCCGTCCGGCGCCGCCG
CCGCCACGGGCGCCTGGGGGTGCACGTAGGGGTGGTGGTGATGGT
GGTGGTACACCGCAGCGGGTACAGCGTTGGCGCCCGCCGCGTGCA
CTGGGTTCCACGAGGCGCCAAACACCGTCGCCTTGGACTGGAAGC
TGCACGGGCTGAAGTCGGGGTGCTCGGCCAGCGTCGCCGCCTGCC
GGGGAGGCTGGCCCAGGGTCCCCGGCGCATAGCGGCCAACGCTCA
GCTCATCCGCGGCGTCGGCGCCCAGCAGGAACGAGTCCACGTAGT
AGTTGCCCAGGGCCCCAGTGGTGGCCATCACCGTGCCCAGCGCCTG
GCCCGCCCGGCCCGACCCACGGAAATTATGAAACTGCAGATTTCAT
GTAACAACTTGGTGGCACCGGGGGGGAAGTACAGTCACCTAATAA
GTTGCCGGCGCCCGCGCCCCCATTGGCCGTGCGCGTCACGTGCCCG
TCCAGCAGAACAATAACGCGTAAATCACTCCGCACGCTATTAATG
GTCCGATGTTTTGCAGTCATAATTTTTATAGCAAAAGCCATATGTTT
TTATGTAAAGGGATCGTGCCGCTCTACGATGGGGTTTGTTTTAATT
GTGGCCAACGACGATTAAAAGATCAAATCTAGCCTTGTCTCTGTAC
TCTCCCGTCTCCCCCCCCATACACACACTTCTTAAGCGGACTATTTT
ATATCACAATTAATCACGCCATCAAGAAGGCGCGGGTCCCGCGTG
CGAGTGCGGCCAGCGGAGCCCCTCACATAAAATTAGACAATAATT
GAAGCCATAAAAAAGCAGCCAAATCGCATTGTCGCTCTACTGTATT
TAAATCTATATTTATGATATTTCATAAGGAGTTATTGTTTCAGAAGC
CACACAGGCTGGCGGGAAGTCGGAAACGACCAACAGATTCGTTTG
CCTCGCCGTGGCTCCCAGCTGTAAAAATTTACGAGGACTTGGAAAG
GTTAGACTGTTGTGTTTGGTTGGCGAGCTCCCTGTAAATAATCCCT
GCGGTCCCCGGGAGAGGCGAGTTTACCCGCGGCCGCCCTCGAAAA
GTCAAATTCAACGCAGGATCCGTCCCAAACGGAGCCGCCGCCGGC
CCTACCAGGGCACTCCAGGCAGGGACCGGCCGCTCAGGGAGTACC
GCGGGTGTAGGTCCCCACAGCTACCCGCCTGGAGCGAGGGGCGCC
CGGGCAACCCTTAAATTCGCCTTTGCTACGAGGACCCCACGGAGG
AGCTGGCCAGGAGGGAGCGGCCAGCCGCCACCAGGGCGAAGGTTT
TGAGGGCCTGGTTGGTTGTGCGGCGCGCTCGGTCCCCGGCCCTCGA
CCCCACGCACACGCGCGCCCAGCCCGCCTTTCTCATCAGCTGGCAA
TCAGGATTCCCAGGCGCAGGCGGCTGGCGACCCAGCCCTGTGCTCC
AGCCTCAGAGGCTCTAACCATGAGCGCTGCAAGCCTGGTTGCGCTC
CGTGAATCCCAGCTGGGGAAAAAACTACAAGTGGCATGAATGGAA
GGCAAGTTCGGTTTGGGAAAAGGCAGCCTCGCCTAAGAGACCCCG
CAGCTCCGGAACCTGGGAGGCCCGCACCGATGTGGCCTGTCCCGG
GGCCGCGTGAGCCTTTCAGGGCTCCTTCCTCCCTTTCCAGCTGCTAC
TCCGGGCCTCGCCTTGGTTACCTACGGGGCCCGGAGACTCGGCG
HOXC4
a. Primers:
HOXC4F: ACCAGGAGCTGTACCCACCAC
HOXC4R: CGCAGAGCGACTGTGATTTCT
b. Amplicon
c. CpG island: Position: chr12: 54411710-
54412131; Band: 12q13.13; Genomic Size: 422
CGCGACTGCTAGAGCTCACACATGCGCAGTGTGGGCCCAGGGCCG
GGCCGCCGAGCAGGAAGCCGGCGCAGCTAGGCGGCCGGCGGGGC
CTGTTAATTGGCAATTAGGGGGGAGGCTGGTGGCTGGTGCGCGTCA
GCCGAGAGGAGAGCGTCTGCCCACCCCCTGCTCCCGCCCCCACTCG
GGCGGATGGAAGGGTGGGAGGTGCCCTGCGTTGGGTGGAGGGTGG
AGGTTGTAGGGTGGGGGTGGGGGATGCTGTACTCAAAAGCCATCT
TGTGCTCAGAGAAAAGAGGCCTACCGGCTTTCCCTTCCGGGGTCCG
GCGCCCCTCACCCCCAGCCGCGGCCATCCCAGCCGGGATGCCCACT
GGACCGGGATGCCCGCTCGCCACGCATGGCTGCTCTGGGCTAGGA
CCTGCCTCGCCTCG
PCDHA13
a. Primers
PCDHA13-G1F: CATGGTGTCGCTCTTCACTG
PCDHA13-G1R: AAGCCAGAGCAGTAGTTGCC
b. Amplicon
c. CpG island: Position: chr5: 140263086-
140264154; Band: 5q31.3; Genomic Size: 1069
CGCCCTGGACCGCGAGAGCGTATCAGCCTATGAACTGGTGGTGAC
CGCGCGGGACGGGGGCTCGCCTTCGCTGTGGGCCACGGCCAGCGT
GTCGGTGGGGGTGGCCGACGTGAACGACAACGCGCCGGCGTTCGC
GCAGCCCGAGTACACGGTGTTCGTGAAGGAAAACAATCCGCCGGG
CTGCCACATCTTCACGGTGTCTGCTCAGGACGCGGACGCACAGGA
GAACGCGCTGGTCTCCTACTCGCTGGTGGAGCGGCGGGTGGGCGA
GCGTGCGCTGTCGAGCTACGTGTCGGTGCACGCGGAGAGCGGCAA
GGTGTACGCGCTGCAGCCGTTGGACCACGAGGAGCTGGAGCTGTT
GCAGTTCCAGGTGAGCGCGCGCGACTCTGGCGTGCCGCCTCTGGGC
AGCAACGTGACGCTGCAGGTGTTCGTGCTGGACGAGAACGACAAC
GCTCCGGCGCTGCTGACGCCCGGGGCTGGCAGCGCGGGAGGCACA
GTGAGCGAGCTGATGCCGCGGTCGGTGGGTGCAGGCCACGTGGTG
GCGAAGGTGCGCGCGGTGGACGCCGATTCGGGCTACAATGCGTGG
CTTTCGTATGAATTGCAGCTGGCGGCGGTCGGCGCGCGCATCCCGT
TCCGCGTGGGGCTGTACACTGGCGAGATCAGCACGACGCGCCCTCT
GGACGAGGTGGACGCGCCGCACCACCGCCTTCTGGTGCTGGTGAA
GGACCACGGTGAGCCCGCGCTGACGGCCACGGCAACGGTGCTGTT
GTCGCTGGTGGAGAGCGGCCAAGCGCCACAGGCTTCGTCGAGGGC
GTCGGCAGGCGCTGTGGGTCCAGAAGCGGCGCTGGTGGATGTCAA
TGTTTACTTGATCATTGCCATCTGCGCGGTGTCCAGCCTGTTGGTGC
TCACGTTGCTGCTGTATACTGCGCTGCGGTGCTCGGCACCGCCCAC
CGAGGGCGCGTGCGCGCCGGGCAAGCCCACTCTAGTGTGCTCCAG
CGCGGCAGGGAGTTGGTCGTACTCGCAGCAGAGGCGGCCGAGGGT
GTGCTCTGGGGAGGGCCCGCATAAGACG
HIC1
a. Primers
HIC1-GF: CTCCCCTCCTCCGTATCACT
HIC1-GR: GGGCTTCCGAGAAGAAAACT
b. Amplicon
c. CpG island: Position: chr17: 1952920-
1962328; Band: 17p13.3; Genomic Size: 9409
cctccggccg gctcagtccc ctccccactc cccaactctg
cccgacgctc cgaccccagc ggggagattc acagtgagaa
tgggtgtggt cgcaagggcc ggaggtaggg ctaggagtgc
cccgacagtg acacccctcc ccctctaaga gcagcgcgga
gccgggggag ggggccgacg aaccacagga agaggcggga
ggggcctggg gtctcctttg gtcaaagctg atatcaaaaa
tataaatttc ccttacccca tcccaccccc gtcccggggt
tctcccccga cccccgagct aaggcacgaa gcagtgaggc
caggtgaggc cgccgagagg tggagccgcc actgtggcga
cgctgcggtt gtcccgggca cagtgggccc tgcgcgccgc
ccccgccgct ccctggggtg cgggccaggg ccgcgcagca
gcgacagagc gggctggcga ggggcgctct aggtgggaga
gaaacggtcg atggtccggc cgtcgggccc ggccgccagg
tgagcgccct ggctcagcac ctcggccgcc ttgtcggggc
tgaggcccag ctcggccgtg aacttggcca gcgggtagag
gctctccagc gccaccttgg ggtcgtgcag gaagtgcgtg
gtctgcgcca gcagctcggc cgcggccgcc ttgtcctgct
gcttcaggct cagctgctcg gccgtgaggc gagccacagc
aaagacgccc tcggggaagt cgagcttgcc cttgccgtcg
gggccgggga cgccggggag cccccccaag cccgccagcg
ccccggccgc gccggccgcg ccccccacgg cgtgcatctt
catgtggctg atgaggttgc gttgctgtgc gaacttgccg
ccgcacacct ggcactcgta gggcttctcg cccgagtgga
tgcgcatgtg ctccgtgagg cggtactggc gcgtgaaccg
catgccgcac gcgtcgcacg cgaagggctt gaggcccagg
tggctgcgca tgtggcgcgt catggtccca cgctgcgtga
acttcttccc gcagatggtg catgggtagg gccgggtcag
ccagtgcgtc ttctcgtgct gccgcagcgt ggccgggtcc
ttgtagctct tgtcgcacga cgcgcagcgg tagggccgca
gcagctctcc caggccaccc ggagccccgg cgaccttgtc
cccgccgcct ccaaaagggg gccctaggcc ggcggcccca
gcggccactt cggccgcctc ggccctgccg tacagcgctt
cctcctcctc cacgtgagcc tccacgtgcg cgttcagctg
ctcagagctg gggaagcect tgccgcacgg aatgcacacg
tacaggttgt caccgaagct ctcgggctcg ccataggcca
ggtgcgggca tgggtagccc tcgaggtggc cgccaggcgg
gctggggtcc tcgctgctac cggtctcctc gctgctgctc
ttgtagtcgt cgccgtcgcc gcccgcgccg ggcccgtcca
ggctgccagg gtagcgcggc ggcggcgcca ggccgagcgg
gggccccccg ggcgagacgg ccgcgtcccc accacgctct
tcgcagcgct cgctggggga gccgcgctcc cggcccagct
cgtcgccata gctacccagg cccggctcgt gcttcatcca
gcgatagagg agactaggcc cgtcggggcg gccggggggc
tcgggtcccg ggctgccgct gccgccgcga aatgggtcgg
aaggcggtgc ggcctcctcc agcttctgga agggcagcgg
cggcagcgac ggcagggcga gaggcggctc cttgtaggcg
gcggggccgg cgctgggagg gctgtccggg cgcgggggca
gctcgcgctc agccagcggc cgctctggcg ccgcggagcc
cggcgggctc ttcttggaca ggtccaggcc acaaagaggg
gagcagcggc gctccgaggc acagagtgcg gcggccgggc
cgggtcccga cgcgtacagc tcggcgcagt gcgtgttgac
cgcggcctct gggcccgagg gcggctccgc ggcaggcggc
ggcggaggcc cgactgggga cgggtagcag gcctggatga
ccggcgtggc ggcccgcagg ccccggcccg gccgaccata
gggcgcgtag ccgccgccgc cgccgccgcc gccccgcagg
tggcagtact tgccgtggcg cttgaggcgt ttcttgcaca
gcgccacgag gtcggggatc tgcaggtagc tggcggcggc
cagcacggcg cccaggctcg gctcagcccc cggggccacg
gccgcggccg cagccgcctc tgcgccgtca gccaggcggc
cggtgtagat gaagtccagc accaggcgga acacggccgg
gctcaccatg tcatggtcca ggttgagcag gttgtcatgc
accaccaggg acttgaggta ggcgctgctg gccgccagca
cgttcttgtg cgcgcggaag agggcgttct gcaccacgat
gatcacgtcg cacaagaagc ccttggtgcg ctggttgttg
agctgcagca gcagctgcct ggagtggccg ggcgcctcca
tcgtgtccag catcgtctgc ccagcacact ctcctgcggg
gacacacacc ggccgggtga gagccgtgcg gcgccctggc
cgcctggccc cagcccggca cttctcccct ccacttcccc
ttccctcagc tgagcggggg catcagccct gcggcctggg
caccggcgaa ggaccggctg ccctctggag tgggagccca
ggccggcccg cccggaccag gagaaggagc aggaggtgag
cggccgccgg tggaggggag gccagggcgg cctgcacgcc
ccagggcacc tggctgggtg ctggggcttc cgagaagaaa
actgttcagg cgcagtgacc cttttggaga cagttacccg
atttaagtaa aatgtccgct tcaggaaaag tcattcaggg
cggagaactt tacccaagta gggagaaagg gagccgagga
accagcgcct cccgcctcgg gagaagttgc cccagttggg
ggaagtgata cggaggaggg gagcgcggtg cccgccctgg
cgccgccctg gccgggggct gtcaaccctc ggtcggggcc
cgggcggcgg ccgcgcgggg agcggaggca gcggctgccg
tggcgggcag agcgcgaagg ccgggcccgg cgcggggagg
gcgttatatc ggggcaggag gctgaggcag gaagcaggtg
ggggggaggg gggagccacg cagctcccag gggagggagg
gggcagcgcc ccgggcgggc acggcgcaca gccggctgcg
gccctgaccc gggcctgcgc cccacccgcg tcccggcctc
ggcctgggcc ctacacgcgc gggcccggcg cctccctccg
cggctccccc ggccccttct cccccggaac tccgccgccc
caaacttggg gaaaagtttt ccaactgcag acagggcggg
aggagtgcgc cggccccagg ccctcggctc gcagctcttc
ctcgcggccc ccaaatccgg cggcagagcc cggagccgag
ccctgagctc ccctgcccgc tgctcgcccg cccgaccccg
ttcccctcct ggcccgcggg gccccgcggc ccgttacctg
cggtcccggc gggccgggct cccctccccg cggcggtggc
agctcttagc cgatgcccca cccgccgctg ccaggccccg
agctgtgcca gggcagcgcc cctgccagcc ccgcccgcca
gctccccttc ccttcccttc ccctcgcctc tccagcccat
gtgcgggcag agccggcccc gggccgctga ccccgccgtg
aacccggcgc ggagccgcgg cccggtggtc ctgagtccga
aagggacgac acccggagcc ctgaacgcca gccgccagcc
gcgatggggc acccgcgcca gaagatgcac ccgaggcggc
cgacgcacga ggaccgggct gtcccgggtc ccccgtccct
cccggtcccc ggctcgagga cccacctggg gggcatgtcg
aaagccccgg gcccggctga cggcggatcc aggggggacg
tggctgcgct gccctccgcc cgccgggccc ccggtcggtc
tgtcctgctg gtccgtcctc cccgcgtcct ggtcgcgtct
cagccccgcc gcgctttccg cacactctta tctggagcgg
cccgggccgg cgggcgctgc tgcggctatg gcgccacctc
gcgggcgcgc agggctctgc gcggcaggcc gctgccttcc
tcccgcgcac ctgagctgga
CDH13
a. Primers
CDH13-GF: GGGAGCGTTAGGAAGGAATC
CDH13-GR: AGGAGAACGCACAGAACGAG
b. Amplicon
c. CpG island: Position: chr16: 82660652-
82661813; Band: 16q23.3; Genomic Size: 1162
CGCGTGCATGAATGAAAACGCCGCCGGGCGCTTCTAGTCGGACAA
AATGCAGCCGAGAACTCCGCTCGTTCTGTGCGTTCTCCTGTCCCAG
GTAGGGAAGAGGGGCTGCCGGGCGCGCTCTGCGCCCCGTTTCTGC
ATTCGGATCGCCCGGCACGGGCAGGGTGAGGGGGCTTTCGGGGGG
TCGGGGCCTCCGGTCGCGGCGGCGAAGACAGATCGGGGCTCGGTA
GGGAGGTCATTCCGAGCCCAGAGATCCTAGGCACCCCCCACACAC
AGGCTCCCACTCTGGCGTGCGTGTGTGTGTGTGTGTGTGTGTGTGT
GTGTGTGTGTGTGTGTGTACGTTCGTTAACGGGAGGAGGAGAGAG
CTCCCAGTCCTTTTTTGCTAGCAGGGGCGACATTCTCGCCCACATC
AAGTGGGGTAACTTTGGTTCCCTCCTCCGGAGGCTCGGTGCATTGG
AGAAAGACTCAGTTAGAGGCGACTCCAACGAGCCGCGGTTTTCCC
CAGCCCAACGCCCAGCGGCCGAAGCGCTGCTCGGGTCCGGATTGC
GGGATGCGGGGCTGGAGAGGCCGAGCAGGCACCACCGACTTCCCA
GGGCGCCCGGGCCCCCTGGTACAGCCCGGCTGCCCGCTGGAAGGC
GCCTCGGGGCAGCAGAGAGCCTCAGCCCGGCTGCTGCTGTCGCTC
AAAGGCGCCGGCGCCGGCCGCACCCGCATCGGGGTCCTTTTGCTCC
CAGACCCCGGGCCCGAAAGGGCCGGAGCGTGTCCCCCGCCAGGGC
GCAGGCCCCAGCCCCCCGCACCCCTATTGTCCAGCCAGCTGGAGCT
CCGGCCAGATCCCGGGCTGCCGCCTCTGCTGCCTTCCCTGAGCGGG
AGCGGAGCGCAGAGAAAAGTTCAAGCCTTGCCCACCCGGGCTGCA
GCTGCTTGTTAACCCTCAGAGCGCCACGGCGCGAGGGAAGGGCAC
GCCAACCAGGAGAGGGGGCGAGGGAGATGCGGTCCGCCTGCAGTC
ACCTCTGCACCTCAGAGATTTCGGGAAGTTTGAGTGCAGGAAAGC
AGCGCTCCGAGGCCAGGCCTGGGGTGCTGGCCGCTGCGGGGGGCA
CGCCCTGCGCTGCTCAGGGGCCTGTGGTTTCGGAGAGCACCCCGAT
CCAGTCCCCCATCGCCTCTCTGGCAGGCG
HOXA4
a. Primers
HOXA4F: TAGTAGGAGGCAGTGGGCTCTC
HOXA4R: AAAACGACAACGCGAGAAAAAT
b. Amplicon
c. CpG island: Position: chr7: 27169573-
27170638; Band: 7p15.2; Genomic Size: 1066
CGGCTGGCTGGCGCGCACATACCCACATCTCACCGCAGCCCGGGTC
AGATGGGGGCTCCCCTCCCGAGGCCCCCTTCCCCTGAGCCTCTCCC
TCCTGACCCCGACCCTCGAACCCAGGCCCAGCCCCGGCCCACCTCC
CGCGCCTCCCAAGCGGCGCCACGTACCGGCGCTGACATGGATCTTC
TTCATCCAGGGGTACACCACGGGCTCCTTGCCCTTCAGGCCCAGCG
GGCTCTTGTCGGCCAAGAGCAGCGGGCACGCGGGGGCGCTGCCCC
CTGCCGGGACGCCTGGGGTGGCGGGGGCCGCCTCGCAGCGCCGCG
GGGCCGCTGGGGGCACGGCGCGAGGCTGCAGGGGCGGCGGCAGCT
GGGGCTGCAGGACGTGGCTCGCATGCAGGCCGTGCGCTGGGCCCT
TGGCTTGCGCCGGGGGCTGCTCGGGCTGGGGCGGCCGCCCGGGGC
TGGCGCCGCCGCGGTAGCCATAGGGGTAGGCGGTGTCCGCGGCCC
CATGCGCGGGGTACAGCGCGGCAGCAGGGTAGGCGGGCTCGCGGG
CGGTCCGCGGCGCGTAGTAGGAGGCAGTGGGCTCTCGGCCGCCGC
CCGCGTGAGGGAGCTGGGGCTGCTGCAGCGGCAGGTGCTGGGTCG
GGGGCGCTGGGGGCTGCTGGTAGCCGGGGCCCCCGCCCGGGCCGC
CGTCTGCGCCGCCCGAGCCGCTGTGCTGCGCGTACTCCTCGAAGGG
AGGGAACTTGGGCTCGATGTAGTTGGAGTTTATCAAAAACGAGCTC
ATGGTCATTAATTTGTGAAGTGCAAAAATACTAATTTTTCTCGCGTT
GTCGTTTTTTCTGGGCTTGCCGAGGCCCCTCCCCCTCCTGCCTCGCT
TCCCATCCCCCTTTCCTCTGCGCCCTTCCCCTCCCCCCGCTGTCAAG
TGCCCACTCCTCCCCCTCCCGCAGACGCCGCCACCAAAGTTCGAGC
CGCTCCTCCCCAGCCCAGCGCGCGCCCCGCCCCGTGCCCCACGTGC
AGCGCCCCCACCAATGGGCGCACCGCGCGCGCGGACCCGGATCAG
GAAACGCGCGGGTGCG
PCDHA6
a. Primers
PCDHA6-G1F: CTGACTGTTGAATGATGGCG
PCDHA6-G1R: TCGGGTACGGAGTAGTGGAG
b. Amplicon
c. CpG island: Position: chr5: 140207726-
140208078; Band: 5q31.3; Genomic Size: 353
CGCTTCTGCTCCTCGCAGCCTGGAAGGTGGGGAGCGGCCAGCTCCA
CTACTCCGTACCCGAGGAGGCCAAACACGGCACCTTCGTGGGCCG
GATCGCGCAGGACCTGGGGCTGGAGCTGGCGGAGCTGGTGCCGCG
CCTGTTCAGGATGGCCTCCAAAGACCGCGAGGACCTTCTGGAGGT
AAATCTGCAGAATGGCATTTTGTTTGTGAATTCTCGGATCGACCGC
GAGGAGCTGTGCGGGCGGAGCGCGGAGTGCAGCATCCACCTGGAG
GTGATCGTGGACAGGCCGCTGCAGGTTTTCCATGTGGACGTGGAGG
TGAGGGACATTAACGACAACCCGCCCTTGTTCCCG
PCDHB15
a. Primers
PCDHB15-G1F: AAGCCTGTTAGCAGAGCACG
PCDHB15-G1R: TCCATCACAGAATAGCGACG
b. Amplicon
c. CpG island: Position: chr5: 140626445-
140627373; Band: 5q31.3; Genomic Size: 929
CGAGCAGAGCATAACCGTGCTGGTGTCGGACGTCAATGACAACGC
CCCCGCCTTCACCCAAACCTCCTACACCCTGTTCGTCCGCGAGAAC
AACAGCCCCGCCCTGCACATCGGCAGTGTCAGCGCCACAGACAGA
GACTCGGGCACCAACGCCCAGGTCACCTACTCGCTGCTGCCGCCCC
GGGACCCGCACCTGCCCCTCACCTCCCTGGTCTCCATTAACACGGA
CAACGGCCACCTGTTCGCTCTCCAGTCGCTGGACTACGAGGCCCTG
CAGGCTTTCGAGTTCCGCGTGGGCGCCACAGACCGCGGCTTCCCGG
CGCTGAGCAGCGAGGCGCTGGTGCGAGTGCTGGTGCTGGACGCCA
ACGACAACTCGCCCTTCGTGCTGTACCCGCTGCAGAACGGCTCCGC
GCCCTGCACCGAGCTGGTGCCCCGGGCGGCCGAGCCGGGCTACCT
GGTGACCAAGGTGGTGGCGGTGGACGGCGACTCGGGCCAGAACGC
CTGGCTGTCGTACCAGCTGCTCAAGGCCACGGAGCCCGGGCTGTTC
GGCGTGTGGGCGCACAATGGCGAGGTGCGCACCGCCAGGCTGCTG
AGCGAGCGCGACGTGGCCAAGCACAGGCTAGTGGTGCTGGTCAAG
GACAATGGCGAGCCTCCGCGCTCGGCCACCGCCACGCTGCAAGTG
CTCCTGGTGGACGGCTTCTCTCAGCCCTACCTGCCGCTCCCAGAGG
CGGCCCCGGCCCAAGCCCAGGCCGACTCGCTTACCGTCTACCTGGT
GGTGGCATTGGCCTCGGTGTCTTCGCTCTTCCTCTTCTCGGTGTTCC
TGTTCGTGGCAGTGCGGCTGTGCAGGAGGAGCAGGGCGGCCTCAG
TGGGTCGCTGCTCGGTGCCCGAGGGCCCCTTTCCAGGGCATCTGGT
GGACGTGAGCGGCACCG
PTPN6
a. Primers
PTPN6-GF: TTCGCATGCGTGAAGTATTATC
PTPN6-GR: AGCTCAGGGACTAAGCCTCA
b. Amplicon
c. CpG island: Position: chr12: 7079501-
7080129; Band: 12p13.31; Genomic Size: 629
CGTGGAGGGGCGCGGGGACAGGGCAAGGGGTTTGGGGGAGGGAC
TGGAAGCGTCCGGCGAGCAGGCGGAGGTTGCTCACCGGTGAACAC
AGATTCGCGCACACCGTAGGCCACGGCGCCGGCCCCCAGCAACAG
CTTCAGGGCCGTGCCCATGCCCCGGGGCCCGGCGGGCAGCCGTCC
CGCCAAGTCCTTCAAGTTCTGGGCCATGTCTGATCTTGAGGCCGGC
GGCACTGGAGGTCAGAAGGGGGTGCCGGCCCGCCTCTACCCCGCT
CCGGCTTAGGTACTGCACCCTTCACACGAGGGTTCGGGCCCGTAAG
GCTGGCGAAAGAAAGGGCAGCGGAAGTGCGCTCCCTTTGAAACCC
TCCCCCTTAGCCCACTACGGACCCGAACTTCGCGCACAGGAATCGC
GCATACGGAAGTCCCGCCCCTTTCTGGAAGGCTGCCCTCCCAGGGA
GGGCAGCGCAAGACAGCAAGTCATCTCCATTTCCTGGCCCACTTTC
AAAATGGCAGCCGGAAGGAAATTTGTGATTAGAAGCCGCGCTGTT
CTTATTTAAGAGCGTTAGCGCAACTTCCGGTATTGTTGCAAGATGG
CCGCGCCCAGTGATGGATTCAAGCCTCGTGAACGAAGCG
APC
a. Primers
APC-GF: GAAGCAGCTGTGTAATCCGC
APC-GR: AAGACAGTGCGAGGGAAAAC
b. Amplicon
c. CpG island: Position: chr5: 112043080-
112043917; Band: 5q22.2; Genomic Size: 838
CGGGACAGAACAGCGAAGCAGTGCCCGGCAAGCGGAGCGCAGCA
CCCATTGCGCCTGCGCATAACAGGCTCTAGTCTCCGGGCTGTGGGA
AGCCAGCAACACCTCTCACGCATGCGCATTGTAGTCTTCCCACCTC
CCACAAGATGGCGGAGGGCAAGTAGCAAGGGGGCGGGGTGTGGC
CGCCGGAAGCCTAGCCGCTGCTCGGGGGGGACCTGCGGGCTCAGG
CCCGGGAGCTGCGGACCGAGGTTGGCTCGATGCTGTTCCCAGGTAC
TGTTGTTGGCTGTTGGTGAGGAAGGTGAAGCACTCAGTTGCCTTCT
CGGGCCTCGGCGCCCCCTATGTACGCCTCCCTGGGCTCGGGTCCGG
TCGCCCCTTTGCCCGCTTCTGTACCACCCTCAGTTCTCGGGTCCTGG
AGCACCGGCGGCAGCAGGAGCTGCGTCCGGCAGGAGACGAAGAG
CCCGGGCGGCGCTCGTACTTCTGGCCACTGGGCGAGCGTCTGGCAG
GTGAGTGAGGCTGCAGGCATTGACGTCTCCTCCCGGCAAAGCTTCC
TCGGCTTTGCCCCGCCGCTGCTCGGGACCCTACGGTGCTCGGCCCG
ACTCTGTGGCTCTCTTCTCTCCATGTCTCACCCTCTCCCCTCCCCGC
ACTCCCCATTCAGGCCTCCAGTTGGCCCCTGGCTTTGCAGGTCCTC
CATTCTCACGCAGTGGATGGGGGTCGCGACGCCCGCCGTCCTCCAC
CTTTCCTGGCTGCTGCTGGAGCTTCGCCCCTGCAAGTGGTGCCCCA
TTCGCGTTAGGTGGGTGGGTCGTCCGCCCTTCCCATTTTAGTCGCTT
CCCCATCTTCCTCG
GSTP1
a. Primers
GSTP1-GF: TTTCCTTTCCTCTAAGCGGC
GSTP1-GR: CTTTCCCTCTTTCCCAGGTC
b. Amplicon
c. CpG island: Position: chr11: 67350929-
67351953; Band: 11q13.2; Genomic Size: 1025
CGGGTGTGCAAGCTCCGGGATCGCAGCGGTCTTAGGGAATTTCCCC
CCGCGATGTCCCGGCGCGCCAGTTCGCTGCGCACACTTCGCTGCGG
TCCTCTTCCTGCTGTCTGTTTACTCCCTAGGCCCCGCTGGGGACCTG
GGAAAGAGGGAAAGGCTTCCCCGGCCAGCTGCGCGGCGACTCCGG
GGACTCCAGGGCGCCCCTCTGCGGCCGACGCCCGGGGTGCAGCGG
CCGCCGGGGCTGGGGCCGGCGGGAGTCCGCGGGACCCTCCAGAAG
AGCGGCCGGCGCCGTGACTCAGCACTGGGGCGGAGCGGGGCGGGA
CCACCCTTATAAGGCTCGGAGGCCGCGAGGCCTTCGCTGGAGTTTC
GCCGCCGCAGTCTTCGCCACCAGTGAGTACGCGCGGCCCGCGTCCC
CGGGGATGGGGCTCAGAGCTCCCAGCATGGGGCCAACCCGCAGCA
TCAGGCCCGGGCTCCCGGCAGGGCTCCTCGCCCACCTCGAGACCCG
GGACGGGGGCCTAGGGGACCCAGGACGTCCCCAGTGCCGTTAGCG
GCTTTCAGGGGGCCCGGAGCGCCTCGGGGAGGGATGGGACCCCGG
GGGCGGGGAGGGGGGGCAGACTGCGCTCACCGCGCCTTGGCATCC
TCCCCCGGGCTCCAGCAAACTTTTCTTTGTTCGCTGCAGTGCCGCCC
TACACCGTGGTCTATTTCCCAGTTCGAGGTAGGAGCATGTGTCTGG
CAGGGAAGGGAGGCAGGGGCTGGGGCTGCAGCCCACAGCCCCTCG
CCCACCCGGAGAGATCCGAACCCCCTTATCCCTCCGTCGTGTGGCT
TTTACCCCGGGCCTCCTTCCTGTTCCCCGCCTCTCCCGCCATGCCTG
CTCCCCGCCCCAGTGTTGTGTGAAATCTTCGGAGGAACCTGTTTCC
CTGTTCCCTCCCTGCACTCCTGACCCCTCCCCGGGTTGCTGCGAGG
CGGAGTCGGCCCGGTCCCCACATCTCGTACTTCTCCCTCCCCGCAG
GCCGCTGCGCGGCCCTGCG
ADAM12
a. Primers
ADAM12-AF: CGCTGAGCTCTTCTAGCCTTTCAT
ADAM12-AR: TCCGCGGATATAAGAACGGTGACT
b. Amplicon
c. CpG island: Position: chr10: 128076156-
128077482; Band: 10q26.2; Genomic Size: 1327
CGGGGCCGCTGCGCGCCCCCCTAAGTGTGTTAGCGGGGGAGGCGG
GGCTGGAAAGGAAACCTGGTGAAGGGCTGGCCCGGAGCCTGGGGT
GGGGATATTCACTGCGGGATAGGGCCAGCAAGAGGACCCGACACG
CATCGTCCCGAGTGACACGTGTAAATGTCAAGATACAGAGACATCT
GCAAATGTCACCCAAGAGGGTGAGGACGGGGGAGCGGTCCCGAG
GCTGTGCCCTCCGGGGCAGGTACTGGCTCCTGTGGGGCTGCGGGCC
AAGTGTCGCCCTTCCCCAAGGAATTGGCACCTGGGGGGGGGGGGT
CGGTCTCGCCGCGCTGGAAGCGCAAGCCCCGGGGCTCCGGAGATG
CGCCGGGGCGCGTCGCCCCTCGGGGCAGCCCTGGACCTCGGCGCG
CCCAGGCGCAGCGTGCGGTGCCCTCGGCGGGGCGGGCAGCGAGCC
GCCCTAGTTCGGCGACTTACCTCGGGCCTCGCAGGGCGCGAGCAG
AGCACCGGCCAGGGCGAGCAGGAGGGCGCGGGCGGGGGACACGG
GCAGCGGGCGCGCTGCCATCGTCGCCGGCCTTCAGTGCAGCAGCTC
TCGGGCCCGGCGGCGAGCGCTGCACCATCCCACGCGGGCGCCGAG
CCGGGGCCGGGCGTCGCGACCGGAGGGATTTCCTGCCTCGGCGAG
TCAGCTCCGGAGCCCTCGCGCAGCGCCCGCGCCGCCGCTGAGCTCT
TCTAGCCTTTCATTTTTAAAAAAGTTTCCCCCCGTGTGTGTGCGTGC
GTGCGCGCGCGCGCGCCGTTCTGGCACAAGCCAGCCTTGACCGTTG
CAATAAATGAGCAAACTGTCCGAGTTGGCCCGGGGACTAGGAAGA
GCGTTAGTGAGAGAAGGCAGGCCTGTGAAATGGATCCACGGCCAG
CAGTCACCGTTCTTATTACCGCGGAACAAATTATTGTCTCCCCCGC
ACCCCCGCCAGTTGGCGGCGTCCCGCGGGTCCTAGAGACCGCTCG
GGTCCCCCCGCCAGGGTCCCGCCCCGAGCCGCGGCTCGCTCACCCC
CGAGGGTGGGCGGCTCAGACGTGGCTCAGTGGCGTCCGGGCGCCC
GGAGCGCACACGTCCCCGCCCCAGGATGATGTGGCCGCAGGGCCC
GGGGCGCCCGGCTGCCAAGCGCACATGCGGCGGCACGGTCCAGCT
TTTCAGGCTGAAGCTGGAAACGATGACTCTGCTACTCGCTCCCCGG
CTCTCTGGGAACCCTCGGAGTGCGGGTCAGGTCTCCACCGCGGCCC
ACAGCCCGGCGCGCGACCCCGCCCGGCCCTAAGCGCCCAAAGGGG
CATCTCTCGCCCG
p16
a. Primers
p16-GF: CTCCTCTTTCTTCCTCCGGT
p16-GR: CCTTCCTTGCCAACGCT
Amplicon
b. CpG island: Position: chr9: 21968359-
21968728; Band: 9p21.3; Genomic Size: 370
CGCAATGGCTTCACGTGCATGTACCCGCCGCCACCGCTCTCCCACA
CCTCCCTGGTCCAGCAGCTAGTCCACTGCCCGCCTGGCTGCTCCAG
GCGCGCCGACCGCTCAAGCGCTCCAGGTCCACCCGGCGGAGGGCA
GAGAAAGCGCGACCGCGCGGCCCGCAGGGTTGCAAGAAGAAAAC
GAGTGTTATATAATGAGTCTCAGTGGTTGCTCACAATGCCAGGCGC
GAAGGCGTGAAGATGTGGCCTTTCCCTTCCCGCATCCCCAGGCATC
TTTTGCACCTGGTGCGGAGTGAGCCAGCCAGCTTGCGATAACCAAA
GGGCGCCTCAGGCTCTGGCGCTCCTCGGCGGAATCCCGTAGCTTCC
CTACG
GABRBA
a. Primers
GABRBA-GF: GGACCTCCCTGACTGTCAAC
GABRBA-GR: CCTCCGGGTAGTCAGAGACA
b. Amplicon
c. CpG island: Position: chr9: 21974579-
21975306; Band: 9p21.3; Genomic Size: 728
CGGAGAATCGAAGCGCTACCTGATTCCAATTCCCCTGCAAACTTCG
TCCTCCAGAGTCGCCCGCCATCCCCTGCTCCCGCTGCAGACCCTCT
ACCCACCTGGATCGGCCTCCGACCGTAACTATTCGGTGCGTTGGGC
AGCGCCCCCGCCTCCAGCAGCGCCCGCACCTCCTCTACCCGACCCC
GGGCCGCGGCCGTGGCCAGCCAGTCAGCCGAAGGCTCCATGCTGC
TCCCCGCCGCCGGCTCCATGCTGCTCCCCGCCGCCCGCTGCCTGCT
CTCCCCCTCTCCGCAGCCGCCGAGCGCACGCGGTCCGCCCCACCCT
CTGGTGACCAGCCAGCCCCTCCTCTTTCTTCCTCCGGTGCTGGCGG
AAGAGCCCCCTCCGACCCTGTCCCTCAAATCCTCTGGAGGGACCGC
GGTATCTTTCCAGGCAAGGGGACGCCGTGAGCGAGTGCTCGGAGG
AGGTGCTATTAACTCCGAGCACTTAGCGAATGTGGCACCCCTGAAG
TCGCCCCAGGTTGGGTCTCCCCCGGGGGCACCAGCCGGAAGCAGC
CCTCGCCAGAGCCAGCGTTGGCAAGGAAGGAGGACTGGGCTCCTC
CCCACCTGCCCCCCACACCGCCCTCCGGCCTCCCTGCTCCCAGCCG
CGCTCCCCCGCCTGCCAGCAAAGGCGTGTTTGAGTGCGTTCACTCT
GTTAAAAAGAAATCCGCCCCCGCCCCGTTTCCTTCCTCCGCG

### DISCUSSION

The present invention is developed upon the prior method disclosed by the United States Patent Application Publication Number 2010/0248228 detecting DNA methylation without bisulfite treatment in clinical setting. Methylation sensitive enzymes are a group of DNA restriction endonucleases that cleave DNA at their recognition sites only when the cytosine of CG is not methylated. The enzymes do not cut the sites containing methylated CG dinucleotides. Although this feature has been utilized to study DNA methylation in developmental biology and in high throughput DNA methylation profiling [16-21], a specific method for tumor cell detection in the clinical setting has not been established. Using multiple methylation sensitive enzymes in this method, unmethylated DNA of normal cells in patient specimens is digested into small fragments; whereas methylated DNA in tumor cells is resistant to digestion and remains intact. These tumor-specific densely hypermethylated regions, often present in CGIs, are differentially amplified by various PCR methods (FIG. 1). In contrast to scattered methylation patter in normal cells including aging cells, the density of aberrant CGI methylation of selected functional genes including tumor suppressor genes in tumor cells is very high [17-20], the PCR target region cannot be cleaved even by a combination of restriction enzymes. To achieve the high specificity, the PCR target regions are carefully selected to contain as many cut sites as possible to ensure complete digestion to avoid false positive results (FIG. 2B and FIG. 2C). As a result, many cuts by multiple restriction enzymes in the target regions in normal DNA produce no amplifiable small DNA fragments (FIG. 1 and FIG. 2A).

Compared with other DNA methylation detection methods [21-29], this method possessed several advantages. First, the method is simple and the whole procedure comprises of three sequential steps: DNA isolation, digestion and a conventional multiplex PCR (FIG. 1). Secondly, the method can be used with a variety of clinical samples including bone marrow aspirate, whole blood, buffy coat, isolated mononuclear cells, plasma or serum, unstained slides, tissue biopsies, or paraffin blocks (data not shown). Thirdly, aberrant CGI methylation is a common phenomenon in cancers including hematopoietic tumors and solid tumors [15-20]. A few markers can detect the majority of B-cell neoplasms by MSR-PCR (FIG. 3). Thus, the method can potentially be used for a wide range of clinical applications in diagnosis and detection of residual circulating leukemia/lymphoma or solid tumor cells, or circulating tumor cell DNA. Fourthly, the analytic sensitivity is high since native genomic DNA, instead of bisulfite-treated DNA, is used as the input DNA. This method can detect as few as 5 leukemic cells in a single-step gel-based PCR (FIG. 2D, upper panel). Depending upon needs in different clinical settings, this method can be modified to have two relative analytic sensitivity levels, 10⁻³ in a single-step PCR, and 10⁻⁶ in a nested PCR (FIG. 2D, middle and lower panels), or a quantitative real-time PCR (FIG. 5). The result was verified independently by a bisulfite-based qMSP method in B-ALL patient specimens (FIG. 4A). Fifthly, the method can be performed as a multiplex PCR to detect methylation in multiple genes in a single tube (FIG. 3B). Thus the clinical sensitivity was increased to over 80% in B-ALL using 3 markers (FIG. 3B), and potentially more by adding markers. With a single marker of DLC-1 gene, the B-ALL patients can be followed in a long period of time and in peripheral blood samples (FIG. 4). Finally, a DLC-1 TaqMan probe-based real-time PCR (qtMSR-PCR) and SYBR Green fluorescence-based real-time PCR (qsMSR-PCR) methods have been developed to quantitatively determine leukemia cells in patient bone marrow specimen with a sensitivity of 10 copies (˜5 leukemia cells) per reaction which has opened a possibility for MRD detection (FIG. 5 and FIG. 8). Using qsMSR-PCR, cancer cells were detectable in 10 out of 94 cancer patient blood samples (FIG. 9).

In addition, the methods herein disclosed were shown to detect hypermethylated loci in both solid tumor cell lines (representing lung, breast, prostate and colon cancers) and hematopoetic cell lines (representing Lymphocytic acute leukemia, acute myeloid leukemia, multiple myeloma).

Like genetic abnormalities in cancer, not all leukemia/lymphoma or carcinoma patients carry the same epigenetic markers. It is critical to select markers that contribute to tumorigenesis, but not just biological “noise” at the genetic and epigenetic levels. In this regard, we selected three DNA methylation markers, DLC-1, PCDHGA12 and RPIB9 as the testing cases, that all play important roles in leukemogenesis and lymphomagenesis. Interestingly, DNA methylation of these three genes demonstrates different specificity in B-cell neoplasms (FIG. 3A). The methylation of DLC-1 and PCDHGA12 was found in almost all B-cell lymphoid tumor cell lines as well as in most B-ALL patient samples, while RPIB9 methylation appears to be only in precursor and germinal center-derived B-cell neoplasms (FIGS. 3A and 3B). The DLC-1 gene encodes a GTPase-activating protein that acts as a negative regulator of Rho signaling [30]. In cancer cells, DLC-1 functions as a bona fide tumor suppressor gene to suppress tumor growth and metastasis [31]. CGI methylation of DLC-1 results in the loss of its expression in many solid tumors and in B-cell neoplasms, thus it can be an invaluable cancer cell biomarker. RPIB9, or Rap2 interacting protein 9, is another GTPase acting protein that regulates the activity of Rap2, a Ras-like GTPase protein [32]. In turn, Rap2 functions as an antagonist to Ras signaling pathways that stimulate cell proliferation [33]. PCDHGA12 encodes a cell surface adhesion protein that plays important roles in cell-cell and cell-matrix interaction and tumor metastasis [34]. Methylation of PCDHGA12 was demonstrated in both lymphoid and myeloid cell lines (FIG. 3A), AML patient bone marrow aspirates, 5 major solid tumor cell lines and the patient samples (data not shown), indicating PCDHGA12 is a potential “universal” tumor marker. Functionally, DLC-1, RPIB9 and PCDHGA12 proteins are linked in their roles by the Ras signaling pathways and cell adhesion. Loss of expression of these functional proteins by CGI methylation may be associated with the increase of tumor cell proliferation and tumor dissemination [17, 18]. DNA methylation of these three genes was also detected in some solid tumors. Transcriptional inactivation of tumor suppressor genes including DLC-1 by CGI methylation may be significant in leukemogenesis and lymphomagenesis and may also serve as an independent prognostic factor [35, 36].

In conclusion, the invention has developed a new type with multiple platforms of PCR-based cancer cell DNA methylation detective method. These platforms include a conventional gel-based PCR, a nested ultra sensitive PCR, a TaqMan probe-based real-time PCR, and SYBR Green fluorescence-based real-time PCR. This unique method was validated by an independent bisulfite-based real-time qMSP assay in clinical patient specimens. Compared with other published DNA methylation detective methods [21-29], this new method demonstrated high sensitivity and specificity, simplicity and quantitative feature. The DNA sample does not require a bisulfite treatment and the background of the assay is very low. In addition, a total of 40 DNA methylation loci in functional genes have been identified with these methods that allows the broad clinical applications for residual circulating tumor cell or tumor DNA detection in both hematopoietic and solid tumors. The invention represents a new type of cancer biomarker detection that can potentially be used in cancer screening, early detection, assessment of therapeutic response, detection of early metastasis and minimal residual disease [37-40].

While the invention has been described in connection with specific embodiments thereof, it will be understood that the inventive device is capable of further modifications. This patent application is intended to cover any variations, uses, or adaptations of the invention following, in general, the principles of the invention and including such departures from the present disclosure as come within known or customary practice within the art to which the invention pertains and as may be applied to the essential features herein before set forth.

Cited references incorporated by reference herein for their respective teachings.
1. Eccles S A, Welch D R: Metastasis: recent discoveries and novel treatment strategies. Lancet. 2007; 369:1742-57.
2. Riethdorf S, Wikman H, and Pantel K: Biological relevance of disseminated tumor cells in cancer patients. Int J. Cancer. 2008; 123:1991-2006
3. Swerdlow S H, Campo E, Harris N L, Jaffe E S, Pileri, S A, Stein H, Thiele J, and Vardiman J W. WHO Classification of Tumours of Haematopoietic and Lymphoid Tissues (4th ed.). Lyon, France: International Agency for Research on Cancer (IARC), Lyon, France, 2008.
4. Kulis M and Esteller M. DNA methylation and cancer. Adv Genet. 2010; 70:27-56.
5. Shivapurkar N, Gazdar A F. DNA methylation based biomarkers in non-invasive cancer screening. Curr Mol Med. 2010; 10(2):123-32.
6. Levenson V V. DNA methylation as a universal biomarker. Expert Rev Mol Diagn. 2010; 10(4):481-8.
7. Craig F E and Foon K A. Flow cytometric immunophenotyping for hematologic neoplasms. Blood. 2008; 111:3941-3967.
8. Evans P A, Pott Ch, Groenen P J, Salles G, Davi F, Berger F, Garcia J F, van Krieken J H, Pals S, Kluin P, Schuuring E, Spaargaren M, Boone E, Gonzalez D, Martinez B, Villuendas R, Gameiro P, Diss T C, Mills K, Morgan G J, Carter G I, Milner B J, Pearson D, Hummel M, Jung W, Ott M, Canioni D, Beldjord K, Bastard C, Delfau-Larue M H, van Dongen J J, Molina T J, Cabecadas J. Significantly improved PCR-based clonality testing in B-cell malignancies by use of multiple immunoglobulin gene targets. Report of the BIOMED-2 Concerted Action BHM4-CT98-3936. Leukemia. 2007; 21:207-214.
9. Bench A J, Erber W N, Follows G A, Scott M A. Molecular genetic analysis of haematological malignancies II: Mature lymphoid neoplasms. Int J Lab Hematol. 2007; 29:229-260.
10. Aster J C and Longtine J A. Detection of BCL2 rearrangements in follicular lymphoma. Am J Pathol. 2002; 160:759-763.
11. Schüler F, Dolken L, Hirt C, Kiefer T, Berg T, Fusch G, Weitmann K, Hoffmann W, Fusch C, Janz S, Rabkin C S, Dölken G. Prevalence and frequency of circulating t(14;18)-MBR translocation carrying cells in healthy individuals. Int J. Cancer. 2009; 124:958-963.
12. Salk J J, Fox E J, Loeb L A. Mutational heterogeneity in human cancers: origin and consequences. Annu Rev Pathol. 2010; 5:51-75
13. Shi H, Guo J, Duff D J, Rahmatpanah F, Chitima-Matsiga R, Al-Kuhlani M, Taylor K H, Sjahputera O, Andreski M, Wooldridge J E, Caldwell C W. Discovery of novel epigenetic markers in non-Hodgkin's lymphoma. Carcinogenesis 2007; 28:60-70.
14. Wang M X., Duff D, Shi H D, Taylor K H, Gruner B A, and Caldwell C W. Detection of minimal residual disease in precursor B lymphoblastic leukemia (B-ALL) patients by a novel epigenetic DNA methylation biomarker, Blood. 2005; 106, Abstract 4524.
15. Laird P W: The power and the promise of DNA methylation markers. Nat Rev Cancer. 2003; 3:253-266.
16. Cotello J F. Aberrant CpG-island methylation has non-random and tumour-type-specific patterns. Nat Genet 2000; 24:132-138.
17. Esteller M. Epigenetics in cancer. N Engl J Med 2008; 358:1148-1159.
18. Galm O, Herman J G, Baylin S B. The fundamental role of epigenetics in hematopoietic malignancies. Blood Rev 2006; 20:1-13.
19. Taylor K H, Pena-Hernandez K E, Davis J W, Arthur G L, Duff D J, Shi H, Rahmatpanah F B, Sjahputera O, Caldwell C W. Large-scale CpG methylation analysis identifies novel candidate genes and reveals methylation hotspots in acute lymphoblastic leukemia. Cancer Res. 2007; 67:2617-2625.
20. Rahmatpanah F B, Carstens S, Guo J, Sjahputera O, Taylor K H, Duff D, Shi H, Davis J W, Hooshmand S I, Chitma-Matsiga R, Caldwell C W. Differential DNA methylation patterns of small B-cell lymphoma subclasses with different clinical behavior. Leukemia. 2006; 20:1855-1862.
21. Singer-Sam J, LeBon J M, Tanguay R L, Riggs A D: A quantitative HpaII-PCR assay to measure methylation of DNA from a small number of cells. Nucleic Acids Res. 1990; 18:687, 1990.
22. Estécio M R, Yan P S, Ibrahim A E, Tellez C S, Shen L, Huang T H, Issa J P. High-throughput methylation profiling by MCA coupled to CpG island microarray. Genome Res. 2007; 17:1529-1536.
23. Beck S, Rakyan V K. The methylome: approaches for global DNA methylation profiling. Trends Genet. 2008; 24:231-237.
24. Herman J G, Graff J R, Myohanen S, Nelkin B D, Baylin S B. Methylation-specific PCR: a novel PCR assay for methylation status of CpG islands. Proc Natl Acad Sci USA 1996; 93:9821-6.
25. Eads C A, Danenberg K D, Kawakami K, Saltz L B, Blake C, Shibata D, Danenberg P V, Laird P W. MethyLight: a high-throughput assay to measure DNA methylation. Nucleic Acids Res. 2000; 28:E32.
26. Eads C A, Laird P W. Combined bisulfite restriction analysis (COBRA). Methods Mol Biol. 2000; 200:71-85.
27. Clark S J, Harrison J, Paul C L, Frommer M. High sensitivity mapping of methylated cytosines. Nucleic Acids Res. 1994; 22:2990-2997.
28. Clark S J, Statham A, Stirzaker C, Molloy P L, Frommer M. DNA methylation: bisulphite modification and analysis. Nat Protoc. 2006; 1:2353-2364.
29. Kristensen L S, Hansen L L. PCR-based methods for detecting single-locus DNA methylation biomarkers in cancer diagnostics, prognostics, and response to treatment. Clin Chem. 2009; 55(8):1471-83.
30. Durkin M E, Yuan B Z, Zhou X, Zimonjic D B, Lowy D R, Thorgeirsson S S, Popescu N C. DLC-1: a Rho GTPase-activating protein and tumour suppressor. J Cell Mol Med 2007; 11:1185-1207.
31. Yuan B Z, Durkin M E, Popescu N C. Promoter hypermethylation of DLC-1, a candidate tumor suppressor gene, in several common human cancers. Cancer Genet Cytogenet 2003; 140:113-7
32. Wang S, Zhang Z, Ying K, Chen J Z, Meng X F, Yang Q S, Xie Y, Mao Y M. Cloning, expression, and genomic structure of a novel human Rap2 interacting gene (RPIP9). Biochem Genet. 2003; 41:13-25.
33. Raaijmakers J H, Bos J L. Specificity in Ras and Rap signaling. J Biol Chem. 2008; 10.1074/jbc.R800061200.
34. Morishita H, Yagi T. Protocadherin family: diversity, structure, and function. Curr Opin Cell Biol. 2007; 19:584-592.
35. Agrawal S, Unterberg M, Koschmieder S, zur Stadt U, Brunnberg U, Verbeek W, Buchner T, Berdel W E, Serve H, Müller-Tidow C. DNA methylation of tumor suppressor genes in clinical remission predicts the relapse risk in acute myeloid leukemia. Cancer Res. 2007; 67:1370-1377.
36. Roman-Gomez J, Jimenez-Velasco A, Barrios M, Prosper F, Heiniger A, Torres A, Agirre X. Poor prognosis in acute lymphoblastic leukemia may relate to promoter hypermethylation of cancer-related genes. Leuk Lymphoma. 2007; 48:1269-1282.
37. Shi H, Wang M X, Caldwell C W. CpG islands: their potential as biomarkers for cancer. Expert Rev Mol Diagn. 2007; 7:519-531.
38. Kagan J, Srivastava S, Barker P E, Belinsky S A, Cairns P. Towards clinical application of methylated DNA sequences as cancer biomarkers: A joint NCI's EDRN and NIST workshop on standards, methods, assay, research and tools, Cancer Res. 2007; 67:4545-4549.
39. Sepulveda A R, Jones D, Ogino S, Samowitz W, Gulley M L, Edwards R, Levenson V, Pratt V M, Yang B, Nafa K, Yan L, Vitazka P. CpG methylation analysis—current status of clinical assays and potential applications in molecular diagnostics: a report of the association for molecular pathology. J Mol Diagn. 2009; 11:266-278.
40. Mulero-Navarro S, Esteller M. Epigenetic biomarkers for human cancer: the time is now. Crit Rev Oncol Hematol. 2008; 68(1):1-11.

1. A method for the diagnosis, prognosis or detection of circulating cancer cells in a subject, comprising:
contacting genomic DNA, obtained from a biological sample of a human subject and having at least one genomic DNA target sequence selected from the CpG island group consisting of HOXD10, COX2, KLF4, SLC26A4, DLC-1, PCDHGA12A, RPIB9, SOX2, CXCR4, HIN1, SFRP2, DAPK1, CD44, CDH1, PGRB, OLIG2, NOR1, SOCS1, RECK, MAFB, p15, HOXD11, HOXA11, HOXA6, HOXA7, HOXD9, HOXA9, HOXC4, PCDHA13, HIC1, CDH13, HOXA4, PCDHA6, PCDHB15, PTPN6, APC, GSTP1, ADAM12, p16, GABRBA, and portions thereof, with a plurality of different methylation-sensitive restriction enzymes each having at least one CpG methylation-sensitive cleavage site within the at least one genomic DNA target sequence, wherein the at least one target sequence is either cleaved or not cleaved by each of said plurality of different methylation-sensitive restriction enzymes;
amplifying the contacted genomic DNA with at least one primer set defining at least one amplicon comprising the at least one target sequence, or the portion thereof, having the at least one CpG methylation-sensitive cleavage site for each of the plurality of different methylation-sensitive restriction enzymes to provide an amplificate; and
determining, based on a presence or absence of, or on a pattern or property of the amplificate relative to that of a normal control, a methylation state of at least one CpG dinucleotide sequence of the at least one target nucleic acid sequence, wherein a method for the diagnosis, prognosis or detection of circulating cancer cells in the human subject is afforded.

2. The method of claim 1, wherein said amplification comprises at least one of standard, multiplex, nested and real-time formats.

3. The method of claim 1, wherein the at least one target sequence comprises the RPIB9 gene CpG island, or a portion thereof.

4. The method of claim 3, wherein the at least one target sequence additionally comprises at least one of the PCDHGA 12 gene CpG island, and portions thereof.

5. The method of claim 3, wherein the at least one target sequence additionally comprises at least one of the DLC-1 gene CpG island, and portions thereof.

6. The method of claim 5, comprising amplification of a plurality of target sequences within the DLC-1 gene CpG island.

7. The method of claim 3, wherein the at least one target sequence additionally comprises the PCDHGA 12 and DLC-1 CpG islands, or portions thereof.

8. The method of claim 1, wherein said methylation sensitive enzyme comprises at least two selected from the group consisting of AciI, HpaII, HinP1I, BstUI, Hha I, and Tai I.

9. The method of claim 8, comprising digestion with Acil, HpaII, HinP1I, and BstUI.

10. The method of claim 1, wherein the at least one genomic DNA target sequence comprises at least 3, at least 4, at least 5, or at least 6 methylation-sensitive restriction sites.

11. The method of claim 1, wherein the at least one genomic DNA target sequence comprises at least four different methylation-sensitive restriction sites, and contacting comprises contacting the at least one genomic DNA target sequence with a respective four different methylation-sensitive restriction enzymes.

12. The method of claim 1, wherein the biological sample comprises at least one of whole blood, buffy coat, isolated mononuclear cells, plasma, serum, bone marrow, and other body fluids (e.g., stool, colonic effluent, urine, saliva, etc.).

13. The method of claim 1, wherein the cancer comprises at least one of hematopoietic tumors, solid tumors, and cutaneous tumors, acute lymphoblastic leukemia (ALL), minimal residual disease (MRD) in acute lymphoblastic leukemia (ALL), acute myeloid leukemia (AML), lung cancer, breast cancer, ovarian cancer, prostate cancer, colon cancer, and melanoma.

14. The method of claim 13, comprising diagnosis or detection of at least one of acute lymphoblastic leukemia (ALL), minimal residual disease (MRD) in acute lymphoblastic leukemia (ALL), and acute myeloid leukemia (AML) in biofluids or tissue samples of either hematopoietic or solid tumors.

15. The method of claim 13, comprising diagnosis or detection of at least one of lung cancer, breast cancer, ovarian cancer, prostate cancer, colon cancer, and melanoma in biofluids or tissue samples of either hematopoietic or solid tumors.

16. The method of claim 1, wherein the relative sensitivity in detecting cancer is one malignant cell or allele in one million normal cells or alleles (10⁻⁶).

17. The method of claim 14, wherein the relative sensitivity in detecting at least one of acute lymphoblastic leukemia (ALL), minimal residual disease (MRD), and acute myeloid leukemia (AML) is one malignant cell or allele in one million normal cells or alleles (10⁻⁶).

18. The method of claim 14, wherein the relative sensitivity in detecting at least one of lung cancer, breast cancer, ovarian cancer, prostate cancer, colon cancer, and melanoma is one malignant cell or allele in one million normal cells or alleles (10⁻⁶).

19. The method of claim 1, wherein the biological sample is from a post-chemotherapy subject.

20. The method of claim 1, wherein the cancer comprises acute lymphoblastic leukemia, and the at least on marker is selected from the group consisting of DLC-1, PCDHGA12A, CDH1, HOXD10, RPIB9, CD44, COX2, SOX2, KLF4, SLC26A, RECK, HOXA9, HOXD11, HOXA6, ADAM12, and HOXC4.

21. The method of claim 1, wherein the cancer comprises chronic lymphocytic leukemia, and the at least on marker is selected from the group consisting of DLC-1, PCDHGA12A, HOXD10, CD44, COX2, HOXA9, HOXA4, HOXD11, and HOXA6.

22. The method of claim 1, wherein the cancer comprises follicular lymphoma, and the at least on marker is selected from the group consisting of DLC-1, PCDHGA12A, CDH1, HOXD10, RPIB9, COX2, KLF4, HOXA9, HOXA6, HOXC4, and SLC26A4.

23. The method of claim 1, wherein the cancer comprises mantle cell lymphoma, and the at least on marker is selected from the group consisting of DLC-1, PCDHGA12A, HOXD10, HOXA9, HOXD11, and HOXA6.

24. The method of claim 1, wherein the cancer comprises Burkett lymphoma, and the at least on marker is selected from the group consisting of DLC-1, PCDHGA12A, CDH1, HOXD10, RPIB9, CD44, COX2, KLF4, HOXA9, HOXD11, HOXA6, HOXC4, and SLC26A4.

25. The method of claim 1, wherein the cancer comprises diffuse large B-cell lymphoma, and the at least on marker is selected from the group consisting of DLC-1, PCDHGA12A, CDH1, HOXD10, RPIB9, COX2, KLF4, HOXA6, and SLC26A4.

26. The method of claim 1, wherein the cancer comprises multiple myeloma, and the at least on marker is selected from the group consisting of DLC-1, PCDHGA12A, CDH1, COX2, KLF4, HOXA9, HOXD11, HOXA6, HOXC4, HOXD10, and SLC26A.

27. The method of claim 1, wherein the cancer comprises acute myeloid leukemia, and the at least on marker is selected from the group consisting of PCDHGA12A, CDH1, HOXD10, CD44, CXCR1, KLF4, SLC26A, CDH13, HOXA9, HOXD11, HOXA6, HOXC4, ADAM12, and SLC26A4.

28. The method of claim 1, wherein the cancer comprises myelodysplastic syndrome, and the at least on marker is selected from the group consisting of PCDHGA12A, SOCS-1, and HIN1.

29. The method of claim 1, wherein the cancer comprises breast cancer, and the at least on marker is selected from the group consisting of DLC-1, PCDHGA12A, HOXD10, RPIB9, COX2, RECK, HOXA11, HOXA7, HOXA9, HOXD9, HOXD11, PCDHB15, PCDHA6, PCDHA13, PTPN6, HIC1, CDH13, GSTP1, ADAM12, p16, GABRBA, and APC.

30. The method of claim 1, wherein the cancer comprises lung cancer, and the at least on marker is selected from the group consisting of PCDHGA12A, HOXD10, HOXA7, HOXA6, HOXA9, PCDHB15, PCDHA6, PCDHA13, PTPN6, GSTP1, and HIC1.

31. The method of claim 1, wherein the cancer comprises colon cancer, and the at least on marker is selected from the group consisting of DLC-1, PCDHGA12A, HOXD10, RPIB9, CD44, COX2, SOX2, CXCR1, SLC26A, RECK, HOXA7, HOXA6, HOXA9, PCDHB15, PCDHA6, PCDHA13, PTPN6, ADAM12, p16, and HIC1.

32. The method of claim 1, wherein the cancer comprises ovarian cancer, and the at least on marker is selected from the group consisting of PCDHGA12A, HOXD10, SLC26A, CDH13, and RECK.

33. The method of claim 1, wherein the cancer comprises prostate cancer, and the at least on marker is selected from the group consisting of PCDHGA12A, HOXD10, COX2, HOXA7, HOXA6, HOXA9, HOXD11, HOXD9, PCDHB15, PCDHA6, PTPN6, HIC1, APC, CDH13, CDH5, HOXA11, GSTP1, p16, GABRBA, and HOXA7.

34. The method of claim 1, wherein the cancer comprises melanoma, and the at least on marker is selected from the group consisting of PCDHGA12A, HOXD10, KLF4, and COX2.


SEQUENCE LISTING (139 sequences)

SEQ ID NO: 1 - DNA, 24 bp, Artificial Sequence
  DLC1-AF primer
taaagagcacagaacaggcaccga

SEQ ID NO: 2 - DNA, 24 bp, Artificial Sequence
  DLC1-AF primer
tgcttgatgtgcagaaagaagccg

SEQ ID NO: 3 - DNA, 24 bp, Artificial Sequence
  DLC1-BF primer
tgttaggatcatggtgtccggctt

SEQ ID NO: 4 - DNA, 24 bp, Artificial Sequence
  DLC1-BR primer
agcgctccctcgtttcgatcttta

SEQ ID NO: 5 - DNA, 25 bp, Artificial Sequence
  DLC1-FF primer
aaatccggagactctgcagaaagcg

SEQ ID NO: 6 - DNA, 20 bp, Artificial Sequence
  DLC1-WF primer
gaaagtgaaccagggcttcc

SEQ ID NO: 7 - DNA, 18 bp, Artificial Sequence
  DLC1-WR primer
taaggcctgcgacccaga

SEQ ID NO: 8 - DNA, 24 bp, Artificial Sequence
  PCDHGA12-AF prime
actcacttctccctcatcgtgcaa

SEQ ID NO: 9 - DNA, 24 bp, Artificial Sequence
  PCDHGA12-AR primer
acctcacttccgcattgactcctt

SEQ ID NO: 10 - DNA, 24 bp, Artificial Sequence
  RPIB9-F primer
tccaggctcctttcctacatcctt

SEQ ID NO: 11 - DNA, 21 bp, Artificial Sequence
  RPIB9-R primer
ggaggaacctgatcaccgtgt

SEQ ID NO: 12 - DNA, 24 bp, Artificial Sequence
  B-actin-AF primer
ggccgaggactttgattgcacatt

SEQ ID NO: 13 - DNA, 24 bp, Artificial Sequence
  B-actin-AR primer
gggcacgaaggctcatcattcaaa

SEQ ID NO: 14 - DNA, 20 bp, Artificial Sequence
  B-actin-BF primer
gagctggtgtccaggaaaag

SEQ ID NO: 15 - DNA, 20 bp, Artificial Sequence
  B-actin-BR primer
gctggaggatttaaggcaga

SEQ ID NO: 16 - DNA, 24 bp, Artificial Sequence
  DLC1QF primer
cccaacgaaaaaacccgactaacg

SEQ ID NO: 17 - DNA, 24 bp, Artificial Sequence
  DLC1QR primer
tttaaagatcgaaacgagggagcg

SEQ ID NO: 18 - DNA, 24 bp, Artificial Sequence
  MSP Q Probe
aagttcgtgagtcggcgtttttga

SEQ ID NO: 19 - DNA, 25 bp, Artificial Sequence
  TaqMan Probe
ccctcgcggtcctcaacgcatcctt

SEQ ID NO: 20 - DNA, 20 bp, Artificial Sequence
  HOXD10F primer
tagccccaagggatctttcc

SEQ ID NO: 21 - DNA, 20 bp, Artificial Sequence
  HOXD10R primer
cacggacaacagcgacatct

SEQ ID NO: 22 - DNA, 295 bp, Artificial Sequence
  HOXD10 CpG island (chr2176982108-176982402)
cgtggcgcggccaagccgcagctctccgctgcccagctgcagatggaaaagaagatgaacgagcccgtgagcggccaggagcccaccaaagtctcccaggtggagagccccgaggccaaaggcggccttcccgaagagaggagctgcctggctgaggtctccgtgtccagtcccgaagtgcaggagaaggaaagcaaaggtcggtatgagcagagttgccaccccagcggggcgcgcagcccgggaacccggcagagagggagtgccggggtgcccagcgccgagccggagcccg

SEQ ID NO: 23 - DNA, 24 bp, Artificial Sequence
  COX2-F primer
tttcttcttcgcagtctttgcccg

SEQ ID NO: 24 - DNA, 24 bp, Artificial Sequence
  COX2-R primer
acgtgacttcctcgaccctctaaa

SEQ ID NO: 25 - DNA, 771 bp, Artificial Sequence
  COX2 CpG island Position chr1186649311- 186650081; Band 1q31.1; Genomic Size 771
cggaaactctgcccgggtgcgtggaaccggagtccccggtgcgcggcgccaggtactcacctgtatggctgagcgccaggaccgcgcacagcagcagggcgcgggcgagcatcgcagcggcgggcagggcgcggcgcgggggtaggctttgctgtctgagggcgtctggctgtggagctgaaggaggcgctgctgaggagttcctggacgtgctcctgacgctcactgcaagtcgtatgacaattggtcgctaaccgagagaaccttcctttttataagactgaaaaccaagcccatgtgacgaaatgactgtttctttccgccttttcgtaccccccacaaatttttccctcctctccccttaaaaaaattgcgtaagcccggtgggggcagggttttttacccacggaaatgagaaaatcggaaacccaggaagctgccccaatttgggagcagagggggtagtccccactctcctgtctgatccctccctctcctccccgagttccaccgccccaggcgcacaggtttccgccagatgtcttttcttcttcgcagtctttgcccgagcgcttccgagagccagttctggactgatcgccttggatgggataccgggggagggcagaaggacacttggcttcctctccaggaatctgagcggccctgaggtccgggggcgcagggaatcccctctcccgccgccgccgccgtgtctggtctgtacgtctttagagggtcgaggaagtcacgtcgggacagactggggcg

SEQ ID NO: 26 - DNA, 24 bp, Artificial Sequence
  KLF4-F primer
aaagtccaggtccaggagatcgtt

SEQ ID NO: 27 - DNA, 24 bp, Artificial Sequence
  KLF4-R primer
cgcaatacagacgcatcacctctt

SEQ ID NO: 28 - DNA, 2912 bp, Artificial Sequence
  KLF4 CpG island Position chr9110249749- 110252660; Band 9q31.2; Genomic Size 2912
cgccccagggggaagtcgtgtgcagccggccggtggccattgctgagagggggtccagcgcccaagtgggtgcacgaagagaccgcctcctgcttgatcttggggcacgtgcgcggcggcccgccgttgtagggcgccaccaccaccgggtggctgccgtcagggctgcctttgctgacgctgatgaccgacgggctgccgtactcgctgccaggggcgctcagcgacgccttcagcacgaacttgcccatcagcccgccacctggcggctgcggctgctgcggcggaatgtacaccgggtccaattctggccgcaggagctcggccacgaagccgcccgaggggctcacgtcgttgatgtccgccaggttgaagggagccgtcggagggggagcggactccctgccatagaggaggcctccgcccgtgccgcccggcgccacgcccgggtcgttcccggcccggatcggataggtgaagctgcaggtggagggcgcgctggcagggccgctgctcgacggcgacgacgaagaggaggctgacgctgacgaggacacggtggcggccactgactccggaggatgggtcagcgaattggagagaataaagtccaggtccaggagatcgttgaactcctcggtctctctccgaggtaggggcgccaggttgctaccgccgcaagccgcaccggctccgccgctctccaggtctgtggccacggtcgccgccgccaggtcataggggcggccgggaagcactgggggaagtcgcttcatgtgggagagctcctcccgccagcgctgcggggacagggcgggagagacctgtcagtggtggtcccctgttgccacccgacatactgacgtgctggcgggccacgcgcgactgcaccgcccagacatggggactggtcaggcaggaagcacccgggaacccagggcgccagcgctgcaatctcggcccactcccgggtcgaagaagaggtgatgcgtctgtattgcgggtgttatgtcctgtctgcccaattgcgtgtgagcgagcgccgcggctggtccctccccctccaggtcccgtggacgtccccggaattggcacaccgaggctctctcggtgcgctctcgccacggggccgcctacgcgctaaactcactctggcccagccagtgtctggggacgcggccacctcccgcccggtggcccgagagcgcccgccctaccgacagcgcgcccggggactggtgaagacccggcttgcgccccaggcggctccgcagtgctcgcaccacgggcatacacagctgagccaaggacacggaagctatcccgggaaggttgcggagtccgcgcggtggccgctccttaccctcgttcagtggctcttggtgaccccaaggctccgcccgcccccaccacacccacgaaaacccaccgggcgttcccggcggcccggagcgatactcacgttattcggggcacctgcttgacgcagtgtcttctcccttcccgccgggccagacgcgaacgtggagaaagatgggagcagcgcgtcgctgacagccatgtcagactcgccaggtggctgcctgcgagcaaggcagggagcggagacaggagagtcaggggcggctttcggccgtcgttccggcgcgtcccaccggtcctcacccctccctgctcccagcgccgcgcgcctcacctacctcattaatgtgggggcccagaaggtcctcggcagcccgaagcagctggggcacctgaaccccaaagtcaacgaagagaagaaacgaagccaaaacccaaaaccccaaattggccgagatccttcttctttggattaaatataacttggaagcgtcttttttaaaaagttcctttgtatacaaaagttcttagaaaagttgtaaacgcaaaaatagacaatcagcaaggcgagtaagtaggtccggtggccgggctgcgctctcttccactcagcagcgtcccccaccactgtcgcggtcgcctcgagtgctgccgtgggcgcaggggctgtggccggggcggtgggcgggcggtgccgccaggtgagactggctgccgtggcgcggagctgcgaactggtcggcggcgcaaggcgcggactccggtgagttgtgtggagcgcgcgcggccatgggcgcgggccacgggcgggtgggagggtggggggccagaggggcgggggagggtcactcggcggctcccggtgccgccgccgcccgccaccgcctctgctccccgcgcgcccgcagacacgttcgttctctctggtcgggaaactgccggccgccggcgcgcgttccttacttataacttccttcgctacagccttttcctccgccttctcccatgccccgcccctccctttcttctctccgccccccccgaggctcccttccatcgttgctatggcagctaaatcaacaaactcggcgcacgtgggggcgggggaggggaaggaggggcgcgggcggggctgggccgggccgtgacgccagccaggcagctggcgggctggagccgagctgacgccggcggcagtggtgtcggcggcggcggcggcgtccgccccagcgcggggcgcgaggaaccgggcgcaggttcggtcgctgcgcgaccagggccgtactcaccgccattgtcggctccctgggttcgaagcccgcgaagactggtggggtcagcgggcggcacggtcacgcgtccgcacccctgctagcatacgcgcttgccgcgctgtctgcgcgctggagaagagcgcgattatccgcgtgactcatccagccctccatctccccctccctctctgcgctcgcaggagtccgctctcgtcgctcagcgccagtgccggtggcggtgccggcgctcggcctgacctcgcacggttcctcgcg

SEQ ID NO: 29 - DNA, 23 bp, Artificial Sequence
  SLC26A4-F primer
agtagccgcccacctctactcta

SEQ ID NO: 30 - DNA, 21 bp, Artificial Sequence
  SLC26A4-R primer
agttagtgggtcccaacggct

SEQ ID NO: 31 - DNA, 1211 bp, Artificial Sequence
  SLC26A4 -  CpG island Position chr7107301206 -107302416; Band 7q22.3; Genomic Size 1211
cgtaaataaaacgtcccactgccttctgagagcgctataaaggcagcggaagggtagtccgcggggcattccgggcggggcgcgagcagagacaggtgagttcgccctgaagatgcccacaccgcccggcccgggctccactcccggggaggcctcgagggttgcggatgggactcttaagtggtcacggatcaggtgggcagggggcagtacagctttctttctgagacgccgagagcgaacaggctgctcggaaaacaggacgaggggagagacttgctcaataagctgaaagttctgcccccgagagggctgcgacagctgctggaatgtgcctgcagcgtccgcctcttggggacccgcggagcgcgccctgacggttccacgcctggcccgggggtctgcacctctcctccagtgcgcacctggagctgcgtcccgggtcaggtgcggggagggagggaatctcagtgtccccttccagccttgcaagcgcctttggcccctgccccagcccctcggtttgggggagatttcagaacgcggacagcgccctggctgcgggccataggggactgggtggaactcgggaagcccccagagcaggggcttactcgcttcaagtttggggaaccccgggcagcgggtgcaggccacgagacccgaaggttctcaggtgcccccctgcaggctggccgtgcgcgccgtggggcgcttgtcgcgagcgccgagggctgcaggacgcggaccagactcgcggtgcaggggggcctggctgcagctaacaggtgatcccgttctttctgttcctcgctcttcccctccgatcgtcctcgcttaccgcgtgtcctccctcctcgctgtcctctggctcgcaggtcatggcagcgccaggcggcaggtcggagccgccgcagctccccgagtacagctgcagctacatggtgtcgcggccggtctacagcgagctcgctttccagcaacagcacgagcggcgcctgcaggagcgcaagacgctgcgggagagcctggccaagtgctgcaggtagcggccgcgcgggcctgcgtagagagaagcggagcggggcgtccacgccttggggagggaagggcgtccccagcgggcgagagtggggtgcgggcggcggagcccctgggcgccagctgcttctcccagaggcccgactttcggtctccggtcctccacgccg

SEQ ID NO: 32 - DNA, 24 bp, Artificial Sequence
  DLC-1AF primer
tgttaggatcatggtgtccggctt

SEQ ID NO: 33 - DNA, 24 bp, Artificial Sequence
  DLC-1AR primer
agcgcaccctcgtttcgatcttta

SEQ ID NO: 34 - DNA, 824 bp, Artificial Sequence
  DLC-1 - CpG island  Position chr812990091- 12990914; Band 8p22; Genomic Size 824
cggtgtcgccgcgcccctcgagccagagccgcgagcccccgcccggctcaaggaggaaagtgaaccagggcttcccttcacgggttgcgaccgatccggagcccgcctggtgcgctggcccgcggtccccaggcaaaaggtaatcaagagtcactcctccaaaattcaaactccctccccaaactgcgagtcctgctatccccacaccacctccaagaaaatccggagactctgcagaaagcgtttaaagagcacagaacaggcaccgacttgacaaggcggggtgacactttctcgcggcgggtcccctccgcagcccgctcccgcggccagcccgacggcaagacgcaagtctagcttacgtgttaggatcatggtgtccggcttctttctgcacatcaagcacggcaggcggcggcggaagcgctgtggggaagtcgaggcaggcggaggcggctcggcttccgcgtcgggacccacggcggcacccgagacgcgcgccctcgcggtcctcaacgcatccttgctcgccgctccctgcccctcgtcacggccccagaaagaaagcggggttttctaaagatcgaaacgagggagcgctcagggagttgggcgagaagtccgtgagccggcgctcctgatgcggagaggtgcggccatgtcctggctgggagcgaagcgccctcgctcgggcagtcggagcgaactgtctcccgcgcgctccgccagccgggccctcccgctgggcccaccccccgaggggcggggccagagcgggcggcaccgcctcctccccgctgtctgggtcgcaggccttagcgacg

SEQ ID NO: 35 - DNA, 20 bp, Artificial Sequence
  PCDHA12-AF3 primer
agtaccccgaattggtgctg

SEQ ID NO: 36 - DNA, 20 bp, Artificial Sequence
  PCDHA12-AR3 primer
tgcttgcacttccatctggt

SEQ ID NO: 37 - DNA, 1017 bp, Artificial Sequence
  PCDHA12 - CpG island Position chr5140256274- 140257290; Band 5q31.3; Genomic Size 1017
cgttggtgctggacagcgccctggaccgcgagagcgtgtcggcctatgagctggtggtgactgcgcgggatgggggctcgccttcgctgtgggccacggctagagtgtccgtggaggtggccgacgtgaacgacaatgcgcctgcgttcgcgcagcccgagtacacagtgttcgtgaaggagaacaacccgccgggctgccacatcttcacggtgtcggcatgggacgcggacgcgcagaagaacgcgctggtgtcctactcgctggtggagcggcgggtgggcgagcacgcactgtcgagctacgtgtcggtgcacgcggagagcggcaaggtgtacgcgctgcagccgctagaccacgaggagctggagctgctgcagttccaggtgagcgcgcgcgacgccggcgtgccgcctctgggcagcaacgtgacgctgcaggtgttcgtgctggacgagaacgacaacgcgccggcactgctggcgactccggctggcagcgcaggaggcgcagttagcgagttggtaccgcggtcggtgggtgcgggccacgtggtggcgaaagtgcgcgcggtggacgctgactccggctataacgcttggctgtcctacgagttgcaaccggcggcggtcggcgcgcacatcccgttccacgtggggctgtacactggcgagatcagcacgacacgcatcctggatgaggcggacgctccgcgccaccgcctgctggtgctggtgaaggaccacggtgagcccgcgctgacgtccacggccacggtgctggtgtcgctggtggagaacggccaggccccaaagacgtcgtcgcgggcctcagtgggcgctgtggatcccgaagcggctctggtggatattaacgtgtacctcatcatcgccatctgtgcggtgtccagcctgctggtgctcacgctgctgctgtacactgcgctgcgttgctcagcgccgcccaccgtgagccggtgcgcgccgggcaagcccacgctggtgtgctccagcgccg

SEQ ID NO: 38 - DNA, 24 bp, Artificial Sequence
  RPIB9-AF primer
tccaggctcctttcctacatcctt

SEQ ID NO: 39 - DNA, 24 bp, Artificial Sequence
  RPIB9-AR
acacggtgatacggttcctcctct

SEQ ID NO: 40 - DNA, 1486 bp, Artificial Sequence
  RPIB9 - CpG island  Position chr787256959- 87258444; Band 7q21.12; Genomic Size 1486
cgcttccgaacacgcgcgtcgaggagggcgttccaggactctgagggagcagcccagctggaccgaggccgcgtcgttcctgggcttactattcccagacccggactcccgattccggagtcacggcccaggacgcgaaaagactctacactggcaccacgctcctccttaggcgggccgtcagtcccgggtgcgggctgcgctggaggctgaggtgggagcgacatggtgtggaggggcaagaaatgtcggcactagacgcgccaagaaggagattctacgagcaattcccccctcgggccattgtgttgctgtttattagcccctgggagggcgtcaggacaaaaggaaccctcctcccttcttagtacttaggcccaaggtcgggtgtgggagccggcgcgctgctttctaggcaggcactgaagctacggcagccacgcaaataggtatcagccgttaaagcttggctacaggcaaggggggggcaataggcccctggcgctgtggggccccgcatcccacaatccccgcggctagcctgtgtggctactggcggcagctagcgggctgcgaaagcgagcccagcgtccttgacagcagcccacgcgtcggggcggggcttgagcccgctgctttaaaaggtccgcgcggccggccccgcccctctggtgccgcgattggatccggcgggggtagcgttgatttgataggcgcagagagggtggggctgcgcacgcgaggccgggggccttgccgctgcctcccgggctggggcacgagtggctgcggagtgtgggtggttgggcgtgaggggccgacgggctcgcgcgcgcgccgtctgctgaggtccctcgggaaggaggagagcgcctgacgccgacccgcaggcgcagcccggcagtcggcggcgcgccgagggcggaggtggtgcgtgcgtgcgtgtgtgtgtgtgtgtgtgtgtgtgtgtgtgtgtgtgtgtggagctcgggtgccaagggcgagccgtcagtccccgggtgcgagtccctgctgtcttccacacccttcctccctccaggctcctttcctacatccttcccgcgcccccacggttgcggaccgagcgagaacccccttaagcaggtgtggggggcgtgcggggtggcacgagacaaaaggggcacgggggtaagcccgccatggcctcccggagcctggggggcctgagcgggatccgcggcggtggcggcggaggcggcaagaaaagcctgagcgcccgcaatgctgcggtggagaggaggaacctgatcaccgtgtgcaggtacggcagcgcagggcgaggggaaccagcctcccgccggggctgagagctctgggcttccgcgcgggtccttgggggtcccgggcatgatgggctgccgcccagtgcccccgcctatgttgcgccagccaaatctgtgagcgcgcagctccttggacaggggcccgggtctggacaccgtcg

SEQ ID NO: 41 - DNA, 24 bp, Artificial Sequence
  SOX2-F primer
acaacatgatggagacggagctga

SEQ ID NO: 42 - DNA, 24 bp, Artificial Sequence
  SOX2-R primer
gccggtatttataatccgggtgct

SEQ ID NO: 43 - DNA, 935 bp, Artificial Sequence
  SOX2 CpG island  Position chr3181430142- 181431076; Band 3q26.33; Genomic Size 935
cgcccgcatgtacaacatgatggagacggagctgaagccgccgggcccgcagcaaacttcggggggcggcggcggcaactccaccgcggcggcggccggcggcaaccagaaaaacagcccggaccgcgtcaagcggcccatgaatgccttcatggtgtggtcccgcgggcagcggcgcaagatggcccaggagaaccccaagatgcacaactcggagatcagcaagcgcctgggcgccgagtggaaacttttgtcggagacggagaagcggccgttcatcgacgaggctaagcggctgcgagcgctgcacatgaaggagcacccggattataaataccggccccggcggaaaaccaagacgctcatgaagaaggataagtacacgctgcccggcgggctgctggcccccggcggcaatagcatggcgagcggggtcggggtgggcgccggcctgggcgcgggcgtgaaccagcgcatggacagttacgcgcacatgaacggctggagcaacggcagctacagcatgatgcaggaccagctgggctacccgcagcacccgggcctcaatgcgcacggcgcagcgcagatgcagcccatgcaccgctacgacgtgagcgccctgcagtacaactccatgaccagctcgcagacctacatgaacggctcgcccacctacagcatgtcctactcgcagcagggcacccctggcatggctcttggctccatgggttcggtggtcaagtccgaggccagctccagcccccctgtggttacctcttcctcccactccagggcgccctgccaggccggggacctccgggacatgatcagcatgtatctccccggcgccgaggtgccggaacccgccgcccccagcagacttcacatgtcccagcactaccagagcggcccggtgcccggcacggccattaacg

SEQ ID NO: 44 - DNA, 23 bp, Artificial Sequence
  CXCR4-F primer
aaactctcgaactgcaggaccca

SEQ ID NO: 45 - DNA, 24 bp, Artificial Sequence
  CXCR4-R primer
taagcgcctggtgactgttcttga

SEQ ID NO: 46 - DNA, 1694 bp, Artificial Sequence
  CXCR4 - CpG island Position chr2136874087- 136875780; Band 2q22.1; Genomic Size 1694
cggtcttaaaacgaaggcccttcggtgcttggggtatattgggcgggagtgtcagaaaatgaacaaacggcacctcctcccccaagcgggcgctcctccggtgtgtgggtctcttgccatcctcgtgtttatcacttggcgcgtttgggacgttagggagcggggcattttcctgggtggagaaggtaacggggtctgcacccgtggtcctcgccccaagtttcatttcctcactctcccgggtggcttcccattaccccgccactgatccagttaacccggccggaggtgggcagctggaagcctccaggcggtgggcacgcggggggccgggtcgtccagccccgggccgccgcggctgcccactacacccacgccaaccgcccgcaagcagcgctgcaggggctccgctgggcgacacgccaggctctgtcccacagggtgctggggagcgactgggcggctccgccgcgagcgtctttgaattgcgcgccgctgcaggaaaccaaaaactccctagcaagagggtttcaaaaggtttctggaaaccaccgacggttaaacatcacaactggactcggagagagccaaacggtttccccacttgcacctgccagtcttcgcggcggcgacctggcagcccaggtgcggtcttaaccgcccccgcccctcaccccgtacccgctcctatccccggagcgcaaatctcagggctggcagctgcgcggtgtcaaaggggaggtcaaaccactccgctgacctctgcacgaccccaaactctcgaactgcaggacccactcgcggccgtggggaagaggcgcgcttcggacggcgggaaggttttccccctcaaacccaaagcgcgcgggcggatcaactcctagctgctgccaccactcgatcccctcagaggatcggcgcggtgggtccacccgcctctcccgccctctgcctactgtgctgggagactggcacagctccgtcggccgcacagagtttaacaaacacgcacccagtgtcaagaacagtcaccaggcgcttaaccccgaagttaaagcgggcgcaatctcctcctgggaactcagcccaggcacgccgccctccgcctctaaattcagacaatgtaactcgctccaagacatccccgcttccccaaggaagagaccggtggtctgagtcccgaggcagcgcgcacgccttctctgcacttgtgcacagaatgttcttacgtttgcaaacagcgtgcaagccgccgcgcgcggcgggactcaagggggagacacatgcagccactggaacgctctttccagtcgtttctcctcgactcacagagaaaaagattccaatcctgctccccccccacccacccgcactatataggcatggtcaagaaaactcctttcggtgacccttttttggagtacgggtacctccaatgtcctggccgcttctgcccgctcggagaggggctgcgctctaagttcaaacgtttgtacatttatgacaaagcaggttgaaactggacttacactgatcccctccatggtaaccgctggttctccagatgcggtggctactggagcactcaggccctcggcgtcactttgctacctgctgccgcagccaacaaactgaagtttctggccgcggccggacttttataaaaacacgctccgagcgcggcgcatgcgccg

SEQ ID NO: 47 - DNA, 24 bp, Artificial Sequence
  HIN1-F primer
gcaaggccacgaggcttcttatac

SEQ ID NO: 48 - DNA, 20 bp, Artificial Sequence
  HIN1-R primer
tcagaccgcaaagcgaaggt

SEQ ID NO: 49 - DNA, 1963 bp, Artificial Sequence
  HIN1 - CpG island Position chr5180017100- 180019062; Band 5q35.3; Genomic Size 1963
cgagctgctcttaaccacgtttattgagaggggccgggggaaggggatggacggtcctccccgcggcggggttttcagccctcgcgggtgggcagcgtcttgtcctcaggtgtagatgctccagtctcggctcagccaaacactgtcagggccccctggaaagcagaagccgagcttgagtgcccccagccctgccaccaagaactcaggcgggggcgcggcagcggccggctctgtggggagcgggagcggggcggttccgctggcgtctccgggggacgcgcacccgcgcggggccatctccgccttccccgcccctgcagctcggatgcgccccacccagttcccacccggagacccgggcttctcccagggacagggcttggaggggcaggacgggaaacagccctgacgtagggccgggacacctctggtgcagttttgaggctggccgggaagggatgcccgcgcaggaagggcacccggggtgcccactttaccagcagggccttcagggccttcacggcccccacggcctggggacccagctcagccacacacttctgggagccctctatgaggtggttcacggggatgcccaggctgctcagcaggagcttcagcgggttgagggtgccgagggggttggccagggtcccggccccggcctccgccgccgactccagcgcagcgacaggctgggccacaggcttggccgagcccactaagaaagcagcagctgcaagcgaacagggaggggtcaccgcctgcgcgccggggtccccagaaggcaggtccaggacgcgcccccgcgggaggcgcccaggaaccgtcgcgccctgcccggctccccgaccgcccctccctcctgcgccgaggcctgccaggtgcgagcccccgggacacaggcgggtctggggaggcggccccgccaggagacgctgcagggtcaccggagtggcctgagggtggcggaaggaccggtgaactctgtgcagggtccgggacaggcccccaagggaggggacactcgcgctgcgccttgcaggatgaggagccggtctccagacggggggcagacgggtgtccccaggccaggggcggcctccatcccggcacgaggctggagacagccctgagagggggaggccgcgggctgcaggcgcggggccccggggtggcggagccctctgggcgccgggcgaggctggaaggacctgggatccacgatcggcgcaggcagcggcgggggcgcagcgggcgccgaggcctcaggccccaccgtgcgcgccaggagcccggggcgctcaccggagctgcaggacagggccacgcagagccccaggagggcggcgagcttcatggcgcgggggctcggggcgcgcggggaacctgcggctgcccgggcaaggccacgaggcttcttatacccggtcctcgcccctccagcgccggcctcgcccgcgctcctgagaaagccctgcccgctccgctcacggccgtgccctggccaacttcctgctgcggccggcgggccctgggaagcccgtgcccccttccctgcccgggcctcgaggacttcctcttggcaggcgctggggccctctgagagcaggcaggcccggcctttgtctccgcgaggcccaccccggcccgcaccttcgctttgcggtctgaccccacgcgcccccctgcagggctgggcccgggtgaggggagcttccctcgcgccagggcaggggcgggggcggcgcagttcctggctccctggtccctgcctctgatcccagaccgtggcaacgtcgggcactgggggtcctcgtgggcgccttctgcgcctggggaggtggaggcgccagggacgatcaggcctcactcccggccgcctccccggccgggccacaggcagccacagtgcaaacagaagtggggcgtttttctgtcttcgaaactagcctcgacg

SEQ ID NO: 50 - DNA, 24 bp, Artificial Sequence
  SFRP2-F primer
gcaattgctgcgcttgtaggagaa

SEQ ID NO: 51 - DNA, 20 bp, Artificial Sequence
  SFRP2-R primer
agtcgcacccagcgaagaga

SEQ ID NO: 52 - DNA, 1315 bp, Artificial Sequence
  SFRP2 - CpG island Position chr4154709513- 154710827; Band 4q31.3; Genomic Size 1315
cgctgctagcgagggggatgcaaaggtcgttgtcctgggggaaacggtcgcactcaagcatgtcgggccaggggaagccgaaggcggacatgaccggggcgcagcggtccttcacctgcacgcagagcgagtggcatggctggatggtctcgtctaggtcatcgaggcagacgggggcgaagagcgagcacaggaacttcttggtgtccgggtggcactgcttcatgaccagcgggatccaagcgccggcctgctccagcacctccttcatggtctcgtggcccagcaggttgggcagccgcatgttctggtattcgatgccgtggcacagctgcaggttggcagggatgggcttgcaattgctgcgcttgtaggagaagtcgggctggccaaagaggaagagcccgcgcgccgagcccaggcagcagtgcgaggcgaggaagagcagcagcagcgagccagggccctgcagcatcgtgggcgcgcgaccccgagggggcagagggagcggagccggggaagggcgaggcggccggagttcgagcttgtcccgggcccgctctcttcgctgggtgcgactcggggccccgaaaagctggcagccggcggctggggcgcggagaagcgggacaccgggaggacagcgcgggcgaggcgctgcaagcccgcgcgcagctccggggggctccgacccgggggagcagaatgagccgttgctggggcacagccagagttttcttggccttttttatgcaaatctggagggtggggggagcaagggaggagccaatgaagggtaatccgaggagggctggtcactactttctgggtctggttttgcgttgagaatgcccctcacgcgcttgctggaagggaattctggctgcgccccctcccctagatgccgccgctcgcccgccctaggatttctttaaacaacaaacagagaagcctggccgctgcgcccccacagtgagcgagcagggcgcgggctgcgggagtggggggcacgcagggcaccccgcgagcggcctcgcgaccaggtactggcgggaacgcgcctagccccgcgtgccgccggggcccgggcttgttttgccccagtccgaagtttctgctgggttgccaggcatgagtgggagagggtgtgtgtgtgtgtgtgtgtgtgtgtgtgtgtgtgtgtgtgtgtgttggggggctgcgtccctggtagccgcgtgtgccctgtgatggagcccgggacctgcccgcccgaggccgcctcggcgaacttcgttttccctcgaatctccagccaccgttcagcagcctgtcg

SEQ ID NO: 53 - DNA, 17 bp, Artificial Sequence
  DAPK1-GF primer
cttgcagggtccccatt

SEQ ID NO: 54 - DNA, 22 bp, Artificial Sequence
  DAPK1-GR primer
ggaacacagctagggagtgagt

SEQ ID NO: 55 - DNA, 1303 bp, Artificial Sequence
  DAPK1 - CpG island Position chr990112515- 90113817; Band 9q21.33; Genomic Size 1303
cgcccgcgttccgggcggacgcactggctccccggccggcgtgggtgtggggcgagtgggtgtgtgcggggtgtgcgcggtagagcgcgccagcgagcccggagcgcggagctgggaggagcagcgagcgccgcgcagaacccgcagcgccggcctggcagggcagctcggaggtgggtgggccgcgccgccagcccgcttgcagggtccccattggccgcctgccggccgccctccgcccaaaaggcggcaaggagccgagaggctgcttcggagtgtgaggaggacagccggaccgagccaacgccggggactttgttccctccgcggaggggactcggcaactcgcagcggcagggtctggggccggcgcctgggagggatctgcgccccccactcactccctagctgtgttcccgccgccgccccggctagtctccggcgctggcgcctatggtcggcctccgacagcgctccggagggaccgggggagctcccaggcgcccgggtgagtagccaggcgcggctccccggtccccccgacccccggcgccagcttttgctttcccagccagggcgcggtggggtttgtccgggcagtgcctcgagcaactgggaaggccaaggcggagggaaacttggcttcggggagaagtgcgatcgcagccgggaggcttccccagccccgcgggccgggtgagaacaggtggcgccggcccgaccaggcgctttgtgtcggggcgcgaggatctggagcgaactgctgcgcctcggtgggccgctcccttccctcccttgctcccccgggcggccgcacgccgggtcggccgggtaacggagagggagtcgccaggaatgtggctctggggactgcctcgctcggggaaggggagagggtggccacggtgttaggagaggcgcgggagccgagaggtggcgcgggggtgccaccgttgccgcaggctggagagagattgctcccagtgaggcgcgtaccgtctgggcgagggcttcattcttccgcggcgtccctggaggtgggaaagctgggtgggcatgtgtgcagagaaaggggaggcggggaggccagtcacttccggagccggttctgatcccaacagaccgcccagcgtttggggacgccgacctcggggtgccgtggtgcccggccccacgcgcgcgcggggctgaggggtcgggggcgtccctggccgcccagctttaacaaagggtgctcctctccaccccgcgaggaggggcagctccggagacccggtcttcagcgagcggggtcttagcgccg

SEQ ID NO: 56 - DNA, 21 bp, Artificial Sequence
  CD44-F primer
ggagaagaaagccagtgcgtc

SEQ ID NO: 57 - DNA, 24 bp, Artificial Sequence
  CD44-R primer
aaacagtgacctaagacggaggga

SEQ ID NO: 58 - DNA, 625 bp, Artificial Sequence
  CD44 - CpG island Position chr1135160376- 35161000; Band 11p13; Genomic Size 625
cggttcggtcatcctctgtcctgacgccgcggggccagcgggagaagaaagccagtgcgtctctgggcgcaggggccagtggggctcggaggcacaggcaccccgcgacactccaggttccccgacccacgtccctggcagccccgattatttacagcctcagcagagcacggggcgggggcagaggggcccgcccgggagggctgctacttcttaaaacctctgcgggctgcttagtcacagccccccttgcttgggtgtgtccttcgctcgctccctccctccgtcttaggtcactgttttcaacctcgaataaaaactgcagccaacttccgaggcagcctcattgcccagcggaccccagcctctgccaggttcggtccgccatcctcgtcccgtcctccgccggcccctgccccgcgcccagggatcctccagctcctttcgcccgcgccctccgttcgctccggacaccatggacaagttttggtggcacgcagcctggggactctgcctcgtgccgctgagcctggcgcagatcggtgagtgcccgccgcagcctgggcagcaagatgggtgcggggtgctcagcgcggacccggcggcagcccctccggctgagtcg

SEQ ID NO: 59 - DNA, 24 bp, Artificial Sequence
  CDH1QF primer
tgagcttgcggaagtcagttcaga

SEQ ID NO: 60 - DNA, 24 bp, Artificial Sequence
  CDH1QR primer
ttcttggaagaagggaagcggtga

SEQ ID NO: 61 - DNA, 1310 bp, Artificial Sequence
  CDH1 - CpG island Position chr1668771035- 68772344; Band 16q22.1; Genomic Size 1310
cgcgtctatgcgaggccgggtgggcgggccgtcagctccgccctggggaggggtccgcgctgctgattggctgtggccggcaggtgaaccctcagccaatcagcggtacggggggcggtgcctccggggctcacctggctgcagccacgcaccccctctcagtggcgtcggaactgcaaagcacctgtgagcttgcggaagtcagttcagactccagcccgctccagcccggcccgacccgaccgcacccggcgcctgccctcgctcggcgtccccggccagccatgggcccttggagccgcagcctctcggcgctgctgctgctgctgcaggtaccccggatcccctgacttgcgagggacgcattcgggccgcaagctccgcgccccagccctgcgccccttcctctcccgtcgtcaccgcttcccttcttccaagaaagttcgggtcctgaggagcggagcggcctggaagcctcgcgcgctccggaccccccagtgatgggagtggggggtgggtggtgaggggcgagcgcggctttcctgccccctccagcgcagaccgaggcgggggcgtctggccgcggagtccgcggggtgggctcgcgcgggcggtgggggcgtgaagcggggtgtagggggtggggtgtggagaaggggtgccctggtgcaagtcgagggggagccaggagtcgtggggacgatcttcgagggaaggagaggggcatccgtagaaataaaggcacctgccatgccaagaaaggtcgtaaataggagtgagggtcccggggataagaaagtgaggtcggaggaggtgggagcgcccctcgctctgaggagtggtgcattcccggtctaaggaaagtggggtactggagaataaagacatctccaataaaatgagaaaggagactgaaagggaacggtgggctaggtcttgagggggtgactcggcggccccctcccgggagttcctgggggctcggcggccgtaggtttcggggtgggggagggtgacgtcgctgcccgcccgtcccggggctgcgggctggggtcctcccccaatcccgacgccgggagcgagggaggggcggcgctgttggtttcggtgagcaggagggaaccctccgagtcacccggttccatctacctttcccccaccccaggtctcctcttggctctgccaggagccggagccctgccaccctggctttgacgccgagagctacacgttcacggtgccccggcgccacctggagagaggccgcgtcctgggcagaggtgagggcgcgctgccggtgtccctgggcg

SEQ ID NO: 62 - DNA, 24 bp, Artificial Sequence
  PGRB-F primer
ataaggcgtgattgagaggcagga

SEQ ID NO: 63 - DNA, 24 bp, Artificial Sequence
  PGRB-R primer
ttgaggaggaggatggctctgagt

SEQ ID NO: 64 - DNA, 1310 bp, Artificial Sequence; identical to SEQ ID NO: 61
  PGRB - CpG island  Position chr1668771035- 68772344; Band 16q22.1; Genomic Size 1310

SEQ ID NO: 65 - DNA, 24 bp, Artificial Sequence
  OLIG2-F primer
tttgaccacgttccctttctccct

SEQ ID NO: 66 - DNA, 24 bp, Artificial Sequence
  OLIG2-R primer
tccgggctaattccgctcaatgaa

SEQ ID NO: 67 - DNA, 10262 bp, Artificial Sequence
  OLIG2 - CpG island Position chr2134395129- 34400245; Band 21q22.11; Genomic Size 5117
gtgggaggggtagaggaaaagcccgcaggggccaggttgggaccccgtaggccgggttagagggcttggacttgatcctgacaggcgacagggagacatattgctacttattatgtgcacagtggccagatctctaaagaaaacaccatcccccacccccaccccccatatagtaaaccaggtggtccgcccagtgctcccagggaggtgatgggaaatcccactccataccctgcggtgaggggttccatgccctccacgtgtgcaactactccgggcccagggaaacactgggccccatccggtaacccccggcccagtcgggtttcccagttcacattataaccaaacggtcttgccagctagacagacagacacccctgacctgtttaccctgatcctctgctctcaggattaatcacaacttgtcgaagggggtggcttccagtggggtggaccgctctgtcaatgccagcgtgtgtctagcatctcctggggtgggggtgtggggaagggaggtgtaggatgaagccctagaagcctcaggcaattgtgatccggtgggctggatactgaagcccacccctgccttgacctcaattttcagtatcttcatctgtaaaatgggaacaacctgccttcctcctagccctaaaggggctgctgtcaagattggctgagatagctgtttgcaagctgagctcaatgaaagttcattgtgtccccctcagtcctatcccaatatcgtctcactgcaaaggtggggggcagcttaacttcaagggcacttcaaggatagccaggtggctgtcagcccagctttccaggatgggagcaggatcttgacagaagggttgactgggaggggcagttgctggtttgggcttcgttaggttgcatttttgtttgttgtcctttcatttccctggggcagcaccccttcctgcaagctccaggccttcctctggaatgctcctagagcccaacctctgctggtgcctgagcttaagccaggccagctaaggggatcctggattcacacggcctcacagtcactcagattgttagcagaagacaaaaattacaaggggagggcgtcatgtgattcttacacaccctccaaatccagcagacaccttggaagccacaggtagcttcaagaaacccattttacggatgagaacctgagatggagaaaggacaactggagatctctgagtctctgagcccacactccctacctccctgcacctccaggcactctgctggcaggatcttgggcaaatgcccacagctctctgagagtcagttttcctgtctgtaaaatgggagtcataccttcctcctatggccggtgagagactaaattaaactatgtctgtcaagacacctgaaactcctggcacaatttaggttgccttcaagtggtcacagttgtcattaggtggaagtcaacaccccaatcattgtaaaggtgcccatataccccaagatccagattacagctctcacagtttattatatacagcgaaaaaacacataacacacctttgcccacatttacatgtattttacggaccatgtttcacatcagtccgcatgcacatctgcacgtgtgtgcattcggcagtatttaccaagcacctgccaagtgccagggcctgtcctccgcacccggcgtgaactgtcctggaccagtcccgggagccgcggttctgaccagccgtgctgaccctggacgactccatgagctgttttgtgagaaagacacgccatttgtttgcagagttctgacttctgaggggtcatgtagcacatgtttggtagccaaacgctgtcattcacgaccaggagcgatggctgcaatgcctttttctttgctttgctttccggtgccgggagccttgcctcccgccgccacccctggtcagctctgcgcaagaacgtcgttctgtttggcagccaggccgagacgcagcctgaatgtgagcaggaactcggagaagggaagggagagaatcagaaagaaggcccgggagggacccgggaagcagtgggaggtctgcgccctggagccccgcgagagcccgccggtttggcacgggctcctcccgggccgcccggcggtccaacaaaggccggccccgacacgcacccggtcttttgtgggagagaaacacaaagaagagggaaaaacacggaggaggccaacagcaccaggacgcgggggccaaccaggaactcccggagccggggcccattagcctctgcaaatgagcactccattccccaggaaggggccccagctgcgcgcgctggtgggaaccgcagtgcctgggacccgcccaggtcgcccaccccgggcgccgggcgcaggacccggacaagtcctggggacgcctccaggacgcaccagggcaagcttgggcaccgggatctaatttctagttattcctgggacggggtggggaggcataggagacacaccgagaggtactcagcatccgattggcaccagggccaagggagcccaggggcgacacagacctccccgacctcccaagctactccggcgacgggaggatgttgagggaagcctgccaggtgaagaaggggccagcagcagcacagagcttccgactttgccttccaggctctagactcgcgccatgccaagacgggcccctcgactttcacccctgactcccaactccagccactggaccgagcgcgcaaagaacctgagaccgcttgctctcaccgccgcaagtcggtcgcaggacagacaccagtgggcagcaacaaaaaaagaaaccgggttccgggacacgtgccggcggctggactaacctcagcggctgcaaccaaggagcgcgcacgttgcgcctgctggtgtttattagctacactggcaggcgcacaactccgcgccccgactggtggccccacagcgcgcaccacacatggcctcgctgctgttggcggggtaggcccgaaggaggcatctacaaatgcccgagccctttctgatccccacccccccgctccctgcgtcgtccgagtgacagattctactaattgaacggttatgggtcatccttgtaaccgttggacgacataacaccacgcttcagttcttcatgttttaaatacatatttaacggatggctgcagagccagctgggaaacacgcggattgaaaaataatgctccagaaggcacgagactggggcgaaggcgagagcgggctgggcttctagcggagaccgcagagggagacatatctcagaactaggggcaataacgtgggtttctctttgtatttgtttattttgtaactttgctacttgaagaccaattatttactatgctaatttgtttgcttgtttttaaaaccgtacttgcacagtaaaagttccccaacaacggaagtaacccgacgttcctcacactccctaggagactgtgtgcgtgtgtgcccgcgcgtgcgctcacagtgtcaagtgctagcatccgagatctgcagaaacaaatgtctgaattcgaaatgtatgggtgtgagaaattcagctcggggaagagattagggactgggggagacaggtggctgcctgtactataaggaaccgccaacgccagcatctgtagtccaagcagggctgctctgtaaaggcttagcaattttttctgtaggcttgctgcacacggtctctggcttttcccatctgtaaaatgggtgaatgcatccgtacctcagctacctccgtgaggtgcttctccagttcgggcttaattcctcatcgtcaagagttttcaggtttcagagccagcctgcaatcggtaaaacatgtcccaacgcggtcgcgagtggttccatctcgctgtctggcccacagcgtggagaagccttgcccaggcctgaaacttctctttgcagttccagaaagcaggcgactgggacggaaggctctttgctaaccttttacagcggagccctgcttggactacagatgccagcgttgcccctgccccaaggcgtgtggtgatcacaaagacgacactgaaaatacttactatcatccggctcccctgctaataaatggaggggtgtttaactacaggcacgaccctgcccttgtgctagcgcggttaccgtgcggaaataactcgtccctgtacccacaccatcctcaacctaaaggagagttgtgaattctttcaaaacactcttctggagtccgtcccctccctccttgcccgccctctacccctcaagtccctgcccccagctgggggcgctaccggctgccgtcggagctgcagccacggccatctcctagacgcgcgagtagagcaccaagatagtggggactttgtgcctgggcatcgtttacatttggggcgccaaatgcccacgtgttgatgaaaccagtgagatgggaacaggcggcgggaaaccagacagaggaagagctagggaggagaccccagccccggatcctgggtcgccagggttttccgcgcgcatcccaaaaggtgcggctgcgtggggcatcaggttagtttgttagactctgcagagtctccaaaccatcccatcccccaacctgactctgtggtggccgtattttttacagaaatttgaccacgttccctttctcccttggtcccaagcgcgctcagccctccctccatcccccttgagccgcccttctcctccccctcgcctcctcgggtccctcctccagtccctccccaagaatctcccggccacgggcgcccattggttgtgcgcagggaggaggcgtgtgcccggcctggcgagtttcattgagcggaattagcccggatgacatcagcttcccagccccccggcgggcccagctcattggcgaggcagcccctccaggacacgcacattgttccccgcccccgcccccgccaccgctgccgccgtcgccgctgccaccgggctataaaaaccggccgagcccctaaaggtgcggatgcttattatagatcgacgcgacaccagcgcccggtgccaggttctcccctgaggcttttcggagcgagctcctcaaatcgcatccagagtaagtgtccccgccccacagcagccgcagcctagatcccagggacagactctcctcaactcggctgtgacccagaatgctccgatacagggggtctggatccctactctgcgggccatttctccagagcgactttgctcttctgtcctccccacactcaccgctgcatctccctcaccaaaagcgagaagtcggagcgacaacagctctttctgcccaagccccagtcagctggtgagctccccgtggtctccagatgcagcacatggactctgggccccgcgccggctctgggtgcatgtgcgtgtgcgtgtgtttgctgcgtggtgtcgatggagataaggtggatccgtttgaggaaccaaatcattagttctctatctagatctccattctccccaaagaaaggccctcacttcccactcgtttattccagcccgggggctcagttttcccacacctaactgaaagcccgaagcctctagaatgccacccgcaccccgagggtcaccaacgctccctgaaataacctgttgcatgagagcagaggggagatagagagagcttaattataggtacccgcgtgcagctaaaaggagggccagagatagtagcgagggggacgaggagccacgggccacctgtgccgggaccccgcgctgtggtactgcggtgcaggcgggagcagcttttctgtctctcactgactcactctctctctctctccctctctctctctctcattctctctcttttctcctcctctcctggaagttttcgggtccgagggaaggaggaccctgcgaaagctgcgacgactatcttcccctggggccatggactcggacgccagcctggtgtccagccgcccgtcgtcgccagagcccgatgacctttttctgccggcccggagtaagggcagcagcggcagcgccttcactgggggcaccgtgtcctcgtccaccccgagtgactgcccgccggagctgagcgccgagctgcgcggcgctatgggctctgcgggcgcgcatcctggggacaagctaggaggcagtggcttcaagtcatcctcgtccagcacctcgtcgtctacgtcgtcggcggctgcgtcgtccaccaagaaggacaagaagcaaatgacagagccggagctgcagcagctgcgtctcaagatcaacagccgcgagcgcaagcgcatgcacgacctcaacatcgccatggatggcctccgcgaggtcatgccgtacgcacacggcccttcggtgcgcaagctttccaagatcgccacgctgctgctggcgcgcaactacatcctcatgctcaccaactcgctggaggagatgaagcgactggtgagcgagatctacgggggccaccacgctggcttccacccgtcggcctgcggcggcctggcgcactccgcgcccctgcccgccgccaccgcgcacccggcagcagcagcgcacgccgcacatcaccccgcggtgcaccaccccatcctgccgcccgccgccgcagcggctgctgccgccgctgcagccgcggctgtgtccagcgcctctctgcccggatccgggctgccgtcggtcggctccatccgtccaccgcacggcctactcaagtctccgtctgctgccgcggccgccccgctggggggcgggggcggcggcagtggggcgagcgggggcttccagcactggggcggcatgccctgcccctgcagcatgtgccaggtgccgccgccgcaccaccacgtgtcggctatgggcgccggcagcctgccgcgcctcacctccgacgccaagtgagccgactggcgccggcgcgttctggcgacaggggagccaggggccgcggggaagcgaggactggcctgcgctgggctcgggagctctgtcgcgaggaggggcgcaggaccatggactgggggtggggcatggtggggattccagcatctgcgaacccaagcaatgggggcgcccacagagcagtggggagtgaggggatgttctctccgggacctgatcgagcgctgtctggctttaacctgagctggtccagtagacatcgttttatgaaaaggtaccgctgtgtgcattcctcactagaactcatccgacccccgacccccacctccgggaaaagattctaaaaacttctttccctgagagcgtggcctgacttgcagactcggcttgggcagcacttcggggggggagggggtgttatgggagggggacacattggggccttgctcctcttcctcctttcttggcgggtgggagactccgggtagccgcactgcagaagcaacagcccgaccgcgccctccagggtcgtccctggcccaaggccaggggccacaagttagttggaagccggcgttcggtatcagaagcgctgatggtcatatccaatctcaatatctgggtcaatccacaccctcttagaactgtggccgttcctccctgtctctcgttgatttgggagaatatggttttctaataaatctgtggatgttccttcttcaacagtatgagcaagtttatagacattcagagtagaaccacttgtggattggaataacccaaaactgccgatttcaggggcgggtgcattgtagttattattttaaaatagaaactaccccaccgactcatctttccttctctaagcacaaagtgatttggttattttggtacctgagaacgtaacagaattaaaaggcagttgctgtggaaacagtttgggttatttgggggttctgttggctttttaaaattttcttttttggatgtgtaaatttatcaatgatgaggtaagtgcgcaatgctaagctgtttgctcacgtgactgccagccccatcggagtctaagccggctttcctctattttggtttatttttgccacgtttaacacaaatggtaaactcctccacgtgcttcctgcgttccgtgcaagccgcctcggcgctgcctgcgttgcaaactgggctttgtagcgtctgccgtgtaacacccttcctctgatcgcaccgcccctcgcagagagtgtatcatctgttttatttttgtaaaaacaaagtgctaaataatatttattacttgtttggttgcaaaaacggaataaatgactgagtgttgagattttaaataaaatttaaagtaaagtcgggggatttccatccgtgtgccaccccgaaaaggggttcaggacgcgataccttgggaccggatttggggatcgttcccccagtttggcactagagacacacatgcattatctttcaaacatgttccgggcaaatcctccgggtctttttcacaacttgcttgtccttatttttattttctgacgcctaacccggaactgcctttctcttcagttgagtattgagctcctttataagcagacatttccttcccggagcatcggactttgggacttgcagggtgagggctgcgcctttggctgggggtctgggctctcaggagtcctctactgctcgatttttagatttttatttcctttctgctcagaggcggtctcccgtcaccaccttccccctgcgggtttccttggcttcagctgcggacctggattctgcggagccgtagcgttcccagcaaagcgcttggggagtgcttggtgcagaatctactaacccttccattccttttcagccatctccactaccctcccccagcggccacccccgccttgagctgcaaaggatcaggtgctccgcacctctggaggagcactggcagcgctttggcctctgtgctctttcctggggtcacctctgtctcctcttggccattgggttctcacaatccaaacccgcgatgcaaatttaggatgtggctgtgaagagagattctgggtggaaataaaaatactttggccttcctggtcaaggaccagggcagatcctgttgtagtctccgtgccccagggctggcctgagaatgagcccctgaaaagacagcgggtacgggcaccgtaagaacatcccctggtccagggtcctctctctgacaatatttttggtggccactggccaccctggaactgggggtgcagaagatttccccagtcagaaccccatttcttgagtcgcatagctgagcctggctcacacaggcaggcaccctttgcttagacttaaagactgctccgtcccctagcaagggacaggcacttcctgctcctccagcagggaatgtcggactgctggccagaacagcagtggcccagggattgggtgctggaggcctagtttttcaccgatgggcctggctttttgcaaaggctgggagggatttggagaggctgagcagctgggggctgaagacgggtggaaagcctcctgcccccaccaccccaacagcgccatgtgaatccaagaagaaggaagggcagggtgtagtcgtttttattctgaaatcccatttgaaatgaaacttgaaaagaattcaaaactgggtccagctgcagccacagacacactcagagggactccaggaggctggaacgtagaccagtgggcgctgagaacctggccggtgggggtaggggtcttgattgcagttttggctcttccacacccactgccaggcaggtgtactggtgcaggctctgagtgtgcttggtgtctgcatagaaggacggttgttgaaaggcaataaatcaagtctttccctccacccctgcacccaagctttcagtagcaaccagccaccagccaggccaggcaagaccagggcctctgaagaaggaggggctgtgtccagccaggctttgggccctcctccatgccagccgcctaaactgtgcacccagctggaggccttgaccacggtgggtgagactggagcagctctggacgtggaggaggaagacactggcacacagtgcacatcccctagaacaggtggctactcgccgagggtggccctggactggtgggggccaaggtagaggactcagccagtggctgggctttgatgtagggcaggagaagactgtgtgcaaccactttgactttggtgggctcttcattggcagtgggctcctcaccaagtagggaagggaaagaggtaactgtttccgggatctgctgcagtcttccctgccacactgcagtcccctctggggagcat

SEQ ID NO: 68 - DNA, 24 bp, Artificial Sequence
  NOR1-F primer
tgaagacgggagctaattggtctg

SEQ ID NO: 69 - DNA, 24 bp, Artificial Sequence
  NOR1-R primer
ttctgcctgggctttcctctgtta

SEQ ID NO: 70 - DNA, 528 bp, Artificial Sequence
  NOR1 - CpG island  Position chr136915797- 36916324; Band 1p34.3; Genomic Size 528
cgatgatgagagggccgggctgctggctgcgggtctggctgagcgggccgggggcctctcacctttgcgggccttgtctcccgggatgttctgggcccgcagccgttggtcgaggatgtaaagcatctccccgcccaagttcaagaagagcagcggtagcgtccgcaccgacatggtgctggaaacgagctggactggtgaagagccccggggttcggtagccagtggcctgaaggccaggccgcagcgtcccaatagtccggttgctggggcaacgccgtgacgggaagagcgagccaatcagaaggcggtttggtgggaggtgccctgaagacgggagctaattggtctgggtggtggaccgtcccggggggattggtccgagccagaggccggcgcggcgttgggcgcggctggggagctgtgcttctgagagtaggtttccctcgaaagggcgagggccgggccagggctgggggtggtctcgacacagccagcccggcgcttgggaccccggccgctggcgcg

SEQ ID NO: 71 - DNA, 24 bp, Artificial Sequence
  SOCS1-F primer
aacacggcatcccagttaatgctg

SEQ ID NO: 72 - DNA, 23 bp, Artificial Sequence
  SOCS1-R primer
tttcgcccttagcgtgaagatgg

SEQ ID NO: 73 - DNA, 2262 bp, Artificial Sequence
  SOCS1 - CpG island Position chr1611348542- 11350803; Band 16p13.13; Genomic Size 2262
cggcctcgtctccagccgagggcgggaggcgcctcgcccctacacccatccgctccctccaacccaggccggggagggtacccacatggttccaggcaagtaataacaaaataacacggcatcccagttaatgctgcgtgcacggcgggcgctgccggtcaaatctggaaggggaaggagctcaggtagtcgcggaggacggggttgagggggatgcgagccaggttctcgcggcccacggtggccacgatgcgctggcggcacagctcctgcagcggccgcacgcggcgctggcgcagcggggcccccagcatgcggcgcggcgccgccacgtagtgctccagcagctcgaagaggcagtcgaagctctcgcggctgccatccaggtgaaagcggccggcctgaaagtgcacgcggatgctcgtgggtcccgaggccatcttcacgctaagggcgaaaaagcagttccgctggcggctgtcgcgcaccaggaaggtgcccacgggctcggcgcgcagccgctcgtgcgccccgtgcacgctcaggggcccccagtagaatccgcaggcgtccaggagcgcgctggcgcgcgtgatgcgccggtaatcggcgtgcgaacggaatgtgcggaagtgcgtgtcgccgggggccggggccgggaccgcggggcacggccgcgggcgcgcgggggccgcgggcgaggaggaggaagaggaggaaggttctggccgccgtcggggctctgctgctgtggagactgcattgtcggctgccacctggttgtgtgctaccatcctacagaaggggccagccggaggggtgggccatagcgtccgggggtgcgctgcgggagagacaaagaggtgagctggggcgctgcggggccgggcaggtgtgcgccggccggacaactccggagggcggcgctcccggcggacccggccctagggggcgagcacggagcaccaagtccgcgcggatccgttcagcctcagtggacacagctagaaaatgggctctgtactccgcggagctcttcccggcgggtgggggctcggtggaggcggagtccggcctccgggcagcaccgagaggggggcgtggagagcagccggttctggctccagccgtccggccccggctcgccgccccgcgcccgccgcctgctggccaggctgggatccgcgcctggtctgggcgatttgggctagggccggagaaaggctgtgctgcgggagccccgcgcgcggggggcggcctgggtggggccggcgagggtcaggggcatcgcggccgcgaccccattctgcagcccccgaggctcgcccgactcctggctgccctggactcccctccctcctccctcccgcctcctcgcccagggcccggctcacctggcggcggggcgcgggacgccgcgggcgggacggcggggggctccggggcgctccggggcggctctcgcgcatgctccggggccaggagccgtgcagctgccacggccgcagctcgctctgttcggcgcccgcccctgcgccagtcttttaaaccggctcggaggcggggctggcgacggcgggaggccccgccccctgccggccccgcccccagctccacttttggtttctctttccgcggtggcgtccggcgaggaccgcttcggccctgtttccctctcttctggaccctcccgcggggccctctgcccgcctgttcgcacctgccccagcacccgcctctcgaggggctctggccccgaccctgcgccttccggccacttctcggacccctccttcggacttggcgaccccgattttgccccgctacctcgggttccactttctgccgccaggccctcttgggacgcgccctgacacaccctcctccgccccagctgtctccacacccgccgggggcagagccctgtcctctcctcccctgcagccagatccccctaggaggccacagaaggtgtccccaaccctgagcctgaccccacccgtagaccccctcctagcccctgctccacccgccgtcgacgccctcagtcgcccgccctgctgtcccgaagccccggccggccgcggtctctggtcttggctcgggcttcccgggaagcggcggcctgaccacaggcttcagaggaacccctggcggcgcgggcgcctccaccccggcccagttcctcggaaactgggcggggccgggcaaggtccctggtggcctcgactgccctccctgcgctcccactacccggctgcg

SEQ ID NO: 74 - DNA, 24 bp, Artificial Sequence
  RECK-F primer
tgagtaacctccagagcaacggtt

SEQ ID NO: 75 - DNA, 24 bp, Artificial Sequence
  RECK-R primer
tttctgacaagcagcagaggcaag

SEQ ID NO: 76 - DNA, 766 bp, Artificial Sequence
  RECK - CpG island Position chr936036799- 36037564; Band 9p13.3; Genomic Size 766
cggggcacgttcccgcccccgggaggttttggaaacactgtgaggcagggggcggggcttgagcgggccgcagccagtcaccaaagggccgggcgctgggggcggggcctcgcgcgagcggcggcggtagcggcggcagcggctgcggccaagctgggtccgagcatcccgcggctctggagccgcccggcccggacatggcgaccgtccgggcctctctgcgaggtgcgctgctccttctgctggccgtggcgggggtcgcggaggtggcagggggcctggctccgggcagtgcgggtgagtaacctccagagcaacggttcgaagctgtcgggagcggccgccacagcgctccaagatggcgcggggcagggggcgggggtgcgcgcgacccccagaccctgcccacgtccggcgaccccgggaccccaggtctcagcgctccagaggctggtgccgaggcggggcgagtgaggaactctctccgccccaagatcttctgggcggtgactcgggtttgaggccttggtctgtcacccaccgacacgggccccctcttcggcactgaccccttcgcttgcctctgctgcttgtcagaaaagggtgcgatgcccccgcccaggatcgtcgcgaggtttagatgggatttcggatacgcagccgccctaccgcggccctagttagttattgttacttgttacttgacccgcacttggttcataacgaccttggtggcggtgagcactgacggtccccacagcccgcg

SEQ ID NO: 77 - DNA, 24 bp, Artificial Sequence
  MAFB-F primer
tcgtgcgttcctgtttctggagat

SEQ ID NO: 78 - DNA, 24 bp, Artificial Sequence
  MAFB-R primer
cgcactttatgcctgtttgagcct

SEQ ID NO: 79 - DNA, 3387 bp, Artificial Sequence
  MAFB - CpG island Position chr2039316551- 39319987; Band 20q12; Genomic Size 3437
ttgaccttgtaggcgtctctctcgcgggccagccgggacacctcctgcttaagctgctccacctgctgaatgagctgcgtcttctcattctccaggtggtgcttctgctggacgcgtttatacctgcaagactgggcgtagccccggttcttcagggtccgccgcttctgcttcaggcggatcacctcgtccttggtgaagccccgcaggtggcggttcagctcgcgcacggacatggacacgagctggtcgtcggagaagcggtcctccacgctgccgttgccgcccgccgccgtcgccgaggccgtcgcgtgcggcccgggcccggggtggctagtgggcagctgttgcgccgggctagcggcgctggacggcggcggcgacgcttggtgatgatggtgatggtgcgggtgagcgtgcgggcccagctcgtcgtgggccacgccggcgcccgggtacgcgtggtgcgggtgagggtggtggtgatggtggtggtggtgagcgccgcgaaagctgtcgaagctttgcagcggctgtggcactgggtgcgagccgatgagcgcttccaccgcgtcctcgggcgtcaggttgagcgcctcggggttcatctgctggtagttgctcgccatccagtacagatcctcgaggtgtgtcttctgttcggtcgggctgaagctgggcgacgagggcacggagctacacggagtgctgagcggtgtggaggacaccgagccggctggctgcaggcgtgtgcagggcctgcccggacgctccgcgcgccccagtggctccttcttcacgtcgaacttgagcaggtcgaagtcgttgacatactccatggccagcgggctggtgggcagctctggccccatgctcagctccgcggccatcgctgaagcgaggcgcagccgccgctgccgcccgggaaactttgcggccggccggagcgcgccgagccaagcgcgggggggaagagcggagaagagctggggaggcggggagcgagggcgcagcgggccggggccgccggccaagcctttgtctggggacgcggcggcgcgccggagagtcccgaggctgcctgcaccgccccagagctctgggctgtgcccgcgcagggaccgggccgggtagagtcgggcggggtggagaggcaagcggagcgcgcggtggggctgaggggaggcgtggggcgagtgcccgttgctcgctctctagctctcttgctcttacgctctctcgctcgcagccgctcgcagctcggcggtgcagctgtgctggatccggcggcgccgcagccttttatcgcctcctgatgtcactggggtgcgggggcccgggcggcccggtgcgcgggccaatagctgcacggcctccgcggcccagcggcgcagggcggggcgcgcctgacagctcccccgccccccgcgtcagctgactggcggcccgagcggccccggagcggcggaggcctggcggagcgctggagcggagtgggacggccagcctgggcccacccccgtaccctgcaggtcccggcccacgcacgctcgcctggagtgcgcgccccacctctaggccaaatcaccgctttcccctcctcgcgcactctcctccctcagttccctttgcaccccacccccatcccgtgtcacccccaaggaggctcagaatgagcgccgggacaacgcctcctgggccctttgttcccaagcggcccccgcccagtgggcgacgctctgtgtgtcctcgcggcttctggccgtgtgtgtcgtgcgttcctgtttctggagatctgcgcgtatttgtatgttggggagggcgggctcgaggctccgagagttgtgttcagacccaactcttaacctcaggggacctttctcaggccaagcgagggcccctcctggcgggtgcagtcgcagagccctgaggttcgactccactggccccgccgctccccgcgttcaccccaccgcacaatgttcacagtgaaggcgacgggaaaagcagcagcccaaaggctctgaattcctcttccccgccacacgcacggaatcctgagcccccggagcctcggggccgaggccggcccgggacggtgctccgagtagctctccactgctggggagccggccctgtttttgtttgaacgttttgtaacgattaagcagatcccggcgtcagcccgccgcggagaggctcaaacaggcataaagtgcgaccccaagtggccactgtgcgcaaaggcgccgcgaccgcccggcccacggccggaaggcttggacggcgcctcgtacccagccaggtctcccctacctggcccaacccaagccagcccagaacgcatactatgtgtgcaccagagcccaggacaggttcccctcgagcgatgtacaggtcctcgggtcccgtcttcgtactcagccgcgagcctcgagccgcgagctccgctctggtcgccccgttgaaattccgtgccccagcgttcgggggtgcccgtcggctgctccctgggccggaaggtcctgggcggaggaaggccggtagccaaaagtggaagcgccacagtgaagcggcccagggccaccgggtgagaaacctccccggagggcagacggggagaccgaagcacaccgcactaggcatccagactgggcttgggagccgcgcaccctccctacccagatccaggatggctagaattaacgggttctttctgagacctcggctcaggcgccgaaaccggatagatcgcgaattcgctggacccggagacccgacccgcctcccgcgtcaccttcttctttctagctttgggcgcgcgcagcgaaaggcaggagaggcgcgcactgggtgagtgagtcccggccgctgtctgcgctggaccagcccgactgacctcgcgcgtaggggtcgcgtgagccacaccggtgcagacgcgcctagattatttttaaatgttagaaggtaaaatatttgcctccaattaatctgaaaactctctattctcttgcgccctcggagaggctggggtacggcgtggtattgggccgcctatttttaataaaatgagtgtattttaactaaaacttaactcaatcttgtggggtggcaaattaaatgctggaagagcgcgtctacaaccctcttcgagaagcgtgctctccgcagaaatgagtcggccgcctggagagagagcctgggcggtgccgctgcgcagcccctgccagtagctgggggttggggactcgcaccttgtaaatgtcctcgtcttgtttgaacgcagtgagagcacactcgtttccagatcactcgggaccgggtgtctcggatctgtgcagactatgtatggctccggcctcaggcggccagggcgggacaagcacg

SEQ ID NO: 80 - DNA, 20 bp, Artificial Sequence
  p15AF primer
acatcggcgatctaggttcc

SEQ ID NO: 81 - DNA, 20 bp, Artificial Sequence
  P15AR primer
ttttcccagaagcaatccag

SEQ ID NO: 82 - DNA, 762 bp, Artificial Sequence
  p15 - CpG island Position chr532585604- 32586365; Band 5p13.3; Genomic Size 762
cgccccatcacgtgaccgcagccccagcgcggcggggccggcgtctcctggctgccgtcacttccggttctctgtcagtcgcgagcgaacgaccaagagggtgttcgactgctagagccgagcgaagcgtgagtgcgcgggaccccctacccctactcctcggggcccccaccctcccagccgggccgtgagctgccttcggccctccactcctctcgccggcaatggccgcgggaaatggcggctctgccttacctcccccttcccctcggcgtccccggcccccttctccgtttctgactccacgcctgacgcgctgtgggcccttccgcggtagactcctgtccccggggagccgagtcgaggcggcgggcgctgcggcccggggcggtagattgagggcggccggggagtgaggagtcgcggggagagagtcgcggcgtccccgggacaatgcggcggcggcctgcctaggtggggcgcgtgcggttacctactcttcccccgcccctcgccctgagcggggcgctctggagactgggagagcggatgcgggcgggagggggccgggggaagaacggctgatgtgcagggggagggaacgcttcgagagaagaaaatggcgcttggtgcaaatcccgccccttcccacgccgtcttctccgcacttcgccgcctcccacgccccctccgaccaacctgtctcccctcgcccgagcggctgctagccacggggttctagcggcttgctggggccgcgcg

SEQ ID NO: 83 - DNA, 21 bp, Artificial Sequence
  HOXD11-G1F primer
gacatttctcttcatggcgtc

SEQ ID NO: 84 - DNA, 20 bp, Artificial Sequence
  HOXD11-G1R primer
cagacggggccacatagtag

SEQ ID NO: 85 - DNA, 599 bp, Artificial Sequence
  HOXD11 -  CpG island  Position chr2176971707 -176972305; Genomic Size 599
cgggcggtggcagatgcgcccagcggtgacagcggccagcggcgcgcaggtgaccggcctgaggcgcagcctggtcagggagcgcccggggagagctggcggcagagggcagccgatccgcccccagcgcgcgcgtctcggcgccaggagccgtcccggggcgtgttggcgagcgttgatatagatataaggacatttctcttcatggcgtcacgtgacataattaccaccagaatcaatcaagatgaattgcacgtcagcgcccggtggggatttttgcttagttgatcctggcccaagcctcttgtgcaatcgatggctcaggttggctgcgcggggagcggccagaggctcgctggcgcgcacgccgcggagtcatgaacgactttgacgagtgcggccagagcgcagccagcatgtacctgccgggctgcgcctactatgtggccccgtctgacttcgctagcaagccttcgttcctttcccaaccgtcgtcctgccagatgactttcccctactcttccaacctggctccgcacgtccagcccgtgcgcgaagtggccttccgcgactacggcctggagcgcgccaagtggccg

SEQ ID NO: 86 - DNA, 21 bp, Artificial Sequence
  HOXA11F primer
aaaactggtcgaaagcctgtg

SEQ ID NO: 87 - DNA, 22 bp, Artificial Sequence
  HOXA11R primer
ccttcagagagtacgccattga

SEQ ID NO: 88 - DNA, 441 bp, Artificial Sequence
  HOXA11 - CpG island Position chr727219310- 27219750, Genomic Size 441
cgcgcggcgacgctcgcgaggcctagcgaatgcgcgttgctttaaattaccataccaatcacttcttgagggtgagtcccctttttctgttatgaaggggagcgggacaagtgaaataatgtaccgtgctgctcttagtatcagaagcgaacaaaggccaagaatcatgctggggttcccggctccccggcggctttgacattgatcggaagtgcgccatctcgtggcggctgcgcgcctaggttgggccggagttccagccccgagccgagagacggaaaccagctccgggcagagagagaaggagagaggagaggatgtgcccagcccgctgctattgagatctcatttttacatctaagaaatcgctgcaaaaccccagccgggtttatagcggcgcattccaaatatgcaaattggccggccccggacgggtttacg

SEQ ID NO: 89 - DNA, 20 bp, Artificial Sequence
  HOXA6F primer
ggaccgagttggactgttgg

SEQ ID NO: 90 - DNA, 20 bp, Artificial Sequence
  HOXA6R primer
gatttgctgctgtcgctttt

SEQ ID NO: 91 - DNA, 2949 bp, Artificial Sequence
  HOXA6 - CpG island Position chr727182614- 27185562; Genomic Size 2949
cgagagccgcgtccccgcggtcgcgtggatttagaaaaaggctggctttaccatgacttatgtgcagcttgcgcatccaggggtagatctggggttgggcgggcggcgccgggctcggctcgctctgcgcactcgcctgctcgctgctggcaggggcgtcctcctcggctccggacgccgtgccaaccccctctctgctgctgatgtgggtgctgccggcgtcggccgaggcgccgctggagttgcttagggagtttttcccgccgtggtggctgtcgctgccgggcgagggggccacggcggagcagggcagcggatcgggctgaggagagtgcgtggacgtggccggctggctgtacctgggctcggcgggcgccgcgctggcgctggcagcgtagctgcgggcgcgctctccggagccaaagtggccggagcccgagcggccgacgctgagatccatgccattgtagccgtagccgtacctgccggagtgcatgctcgccgagtccctgaattgctcgctcacggaactatgatctccataattatgcaactggtagtccgggccatttggatagcgaccgcaaaatgagtttacaaaataagagctcatttgttttttgatatgtgtgcttgatttgtggctcgcggtcgtttgtgcgtctatagcacccttgcacaatttatgatgaattatggaaatgactgggacatgtacttggttccctcctacgtaggcacccaaatatggggtacgacttcgaatcacgtgcttttgttgtccagtcgtaaatcctgcctgatgacctctagaggtaaactcgtgcactaataggggagttgggtggaggcgaggggggtggcgcgcgcgccccgggcgcgtgcccgccgccagttgccgccgttcagccggactcgagcgccacccgctggaggcagggctcatcgcccagcttccgaccgggggctgcaagggccggggtcgaattgaggttacagcccattatggcaaaattattgcatttccctcgcagttccattaggatgtaccaattgttaggccgtcagctgccgatcgcgcgcccggcgaggatgcagaggattggggggaggtggtgacttgcattttatttacaacaactttatttcccccgttttgcagcccctcttatttttgtgtcgaggttggggtcggtactgaccgtcctgccagcagctctgaattttgaaaatacagatatcaccttcggggaagggggaaagccatttagccaattggagaaataaatcctgcccgcagcagcagcagctacaattacggctctgtttttgcgagcgcatgagggacagtgtccctgccgctcttaaatgacaggcgtctattaaagatagcttttgtgtagtgtttctccaaggcgaggtcaaattccatacacttttataaccgtagtcgatttttctttcgtgtgaatatggttttcgtgtcattagtttgcgatttgatttgcttacgtatccagcctggaaaatcttcatcacagggtccggttcctcgagccagccgggccccaagtcggagggttctccttgaacccagcgagtgggcccaggctccctgcagccacagaggctgcctggggtctggggatccgtggggcgggttactggggtcttgcttagacctccaggagtaaaatgagggcgataatggaagcattccttggcagtgcctagtatctctgtagttattttccacggctccgaaagactcaagtaaatcacaaatatagctgagaggcaagtggagtctccccgctggaggcccggcgttgcaggcgcccctggcacgtctggaagccaggactctggcggctcccatggccctgggcccctcgttgggtcctgaacgctgctgtggcggcgacgcgggcgctatcggaggctgggagcgggaatccggagccgggagcctaccccgggctgtaatgttccacccgcgcccaggttaactcgcctcggctgaggctgcttctcttccactgacggttgcacacgcgggaccgagagactgggctctgttggggccccctttgttcctcgagcttccttcctgttctgggaggcggcttgggaggccgcgacaaggccgggctccagctcttagaccccctctttccactggccagagatgatttgatgatgcccttcgggacttactggcgagggacttaggcagagacgcccagacacgaaacggggctcggcccagggctctttcctccccagcagccccgcgtcccgaggtcggggagctcagagacactagcacaggagccccagacgcattcagggcgcaccccagaactccggagccggtttgggcatccttgtggagcgggactgggtgtgtgcagtgcgccccgctccaccgctggtattggctgtgtgtgaggttttgttttgttttgttttgttttgttttgttttgttttgttttgttttgtaagaaataaatgcacagacgcttgcaaagctccgggctcccctgaagctgcggaagcccccagatgggagcaggcggggagaaaagttggggaacaggcgagggcaagggggcaaagccgaaggaggttgcagcgctggcctggtccctgcccaggcatctactcgcccgcctttgcctctgagtcctccccgctgggctgcgtggaattgatgagcttgttttcctttttccacttcatgcggcggttctggaaccagatcttgatctggcgctcggtgaggcagagcgcgttggcgatctcgatgcggcggcgccgtgtcaggtagcggttgaagtggaactccttctccagctccagtgtctggtagcgcgtgtaggtctggcggcctcggcgcccatggctcccatacacagcacctacgagcagaaacggccgggcgccg

SEQ ID NO: 92 - DNA, 20 bp, Artificial Sequence
  HOXA7F primer
acgcaaaggggctctgataa

SEQ ID NO: 93 - DNA, 20 bp, Artificial Sequence
  HOXA7R primer
aaagctgccggacaacaaat

SEQ ID NO: 94 - DNA, 966 bp, Artificial Sequence
  HOXA7 - CpG island Position chr727195602- 27196567; Genomic Size 966
cgcaatggcgcctccgctccaattaaaaccagaaaggctgcgccgggagtcacggggctaccggctcgcaacagcctggctccgctcttccggccccgcgccccgcgctccgcgctccccagcgctgcgctccccgctcccggtcccgctccgccagcctggcccgcctagcgactgcgcctacctgaagaccgcatccaggggtagatgcggaaattggcctcagccgcgccatgcagcgcgccctcgtccgtcttgtcgcaggcgcctttggcgaggtcactgcagagcccggggatgttttggtcgtaggaggcgcagggcaggttgccgtaggcgtcggcgcccaggccgtagccggacgcaaaggggctctgataaagggggctgttgacattgtataagcccggaacggtcgaggcgaaggcgccggcgcccgccccgtagccgcttctctgtgagttgggagcaaaggagcaagaagtcggctcggcattttggaacagagaagcccccgccgtatatttgctaaaaagcgcgttcacataatacgaagaactcataattttgacctgtgatttgttgtccggcagctttcagtgtcggttttacgaggtagagtgatatatgataacattacacccccagatttacaccaaaccccattttcttttggacggagctcgccgcagcacgtgaccgcccacatgaccgcctccgccaatctcagcagtcctcacaggtggtctcgctccgcagggcccgcagccgcctagaatggaagggcaagaggctcaaatatgcggccaaagaatccgcccgcgcccggcgggcctggcgcgtcccgcggaaaaagacctggaggctccgcgggagcgcccagctggcggccaacctccgcactggggtctgcggacgccaggcggcccggccccacgcagcaccccccaccccgcccccccgccg

SEQ ID NO: 95 - DNA, 20 bp, Artificial Sequence
  HOXD9-G1F primer
ctaattgcggcgcttatgtt

SEQ ID NO: 96 - DNA, 20 bp, Artificial Sequence
  HOXD-G1R primer
tggcctataagcgagtccac

SEQ ID NO: 97 - DNA, 1867 bp, Artificial Sequence
  HOXD9 - CpG island  Position chr2176986425- 176988291; Genomic Size 1867
cggccgaattttttagacattttgggagtctcctccgaggcctttaagtgcgaaccgcgcgaagcggccctgcccggggagactcgctgaggcagggctgaggcggcgggcgggagcaagctgctctagcatttgggttctgccctgtggcgtgttctcttccagggcctttccagcatcatcggagaagacgaagcaccctggccgccactgtccgtgctgcgccaactcgcccggccgcccgcccttccgagggcaggcagaagcccctctgtgtcctccaccgccgcgccccggctcgcccctcgggccgcggcgtgtgcccagcctcacgtcggggtgtgtgtggccgcgcgggcgtgtgtgagtgtggcagggggagggggccctccgatctgctccatccgtccgttttattagggacacattaatctataatcaaatacacctcataaaatttttattgaaaggcataatatcattacagaggtcttccacctgttttaaacaacacgacaagctgtgagcaagcgtgtgtgtggggatgtgtggggaggggtgggtgtgagtagggagagaggcgaggggagaacagctcccctcgggcgctaggggccgccccgagggcccgcctgcctcgggcgacaccggcctggcgcccccgcggccgctccgtgtgccctggactcgccgcccgcggctcggaagctggagagtcagcgacggggcccgactgcgggaccgagggctgcaagaagaagcgaacaaatagtccccagcgcctcctctggatgcggtcgcgtctgtggtcctggcagccgctgggcgggccaggccaggtcgggccgggccgagccgggcacatggacctgggcctgcgggctctaattgcggcgcttatgttgatgattttttttttaatcacagcagcccccagtttagcggactgatttactcccggtattggtaaatatgatcacgtgggccgcgcgaccaatggtggaggctgcagcctgcgaactagtcggtggctcgggcgccggcggggagctgctcggcggcggacagtgtaatgttgggtgggagtgcgggacgcctcaaaatgtcttccagtggcaccctcagcaactactacgtggactcgcttataggccatgagggcgacgaggtgttcgcggcgcgcttcgggccgccggggccaggcgcgcagggccggcctgcaggtgtggctgatggcccggccgccaccgccgccgagttcgcctcgtgtagttttgcccccagatcggccgtgttctctgcctcgtggtccgcggtgccctcccagcccccggcagcggcggcgatgagcggcctctaccacccgtacgttcccccgccgcccctggccgcctctgcctccgagcccggccgctacgtgcgctcctggatggagccgctgcccggcttcccgggcggtgcgggcggtggcggtggtggtggaggcggcggtccgggccgcggtcccagccctggccccagcggcccagccaacgggcgccactacgggattaagcctgaaacccgagcggccccggcccccgccacggccgcctccaccacctcctcctcctccacttccttatcctcctcctccaaacggactgagtgctccgtggcccgggagtcccaggggagcagcggccccgagttctcgtgcaactcgttcctgcaggagaaggcggcagcggcgacggggggaaccgggcctggggcagggatcggggccgcgactgggacgggcggctcgtcggagccctcagcttgcagcgaccacccgatcccaggctgttcgctgaaggaggaggagaagcagcattcgcagccg

SEQ ID NO: 98 - DNA, 20 bp, Artificial Sequence
  HOXA9-G1F primer
agcaggaacgagtccacgta

SEQ ID NO: 99 - DNA, 20 bp, Artificial Sequence
  HOXA9-G1R primer
tgcaaaacatcggaccatta

SEQ ID NO: 100 - DNA, 2547 bp, Artificial Sequence
  HOXA9 - CpG island Position chr727203916- 27206462; Band 7p15.2; Genomic Size 2547
cggagctgggcaagccgtcagggcgccctaaggccgctgatcacgtctgtggcttatttgaataatctgtcatggggacccttgtggcccgggtcgcccgcagcctcatcttggcaggatttacgccgccactggccgaaggcaagaagtggaaggaatcggccgtctcccccagcgtcccagctccggctgccctggctgccgccgctcacggacaatctagttgtacaaaaggctctctgggctgcactgctttcgaagaacggcccaaagtatctcggtcctgggcctgggcagccaaggagaggggcggccagtcttggctcgtcccgaagtgcccgccccgccccctctcgctgcagcagccgcctcctctcccgtagccctgcgggccgctcttcactgctctccagacttggggccctatctgaggcgtcccaaacaccaacttctggctcctggccccaactcgagaggcttccagcgaggacgaaggcaggctcgagagaaacctggcgggccagcagatccgggaggccggcgtggaggcggcggcggatttgaagggaggagacacttactgggatcgatggggggcttgtctccgccgctctcattctcagcattgttttcagagaaggcgccttcgctgggttgtttttctctatcaactggaggagaaccacaagcatagtcagtcagggacaaagtgtgagtgtcaagcgtgggacagtcaccccttctggccgacagcggttcaggtttaatgccataaggccggctggagggcaagcccgcgaaggagagcgcaccgggcgtgggctccagccaggagcgcatgtacctgccgtccggcgccgccgccgccacgggcgcctgggggtgcacgtaggggtggtggtgatggtggtggtacaccgcagcgggtacagcgttggcgcccgccgcgtgcactgggttccacgaggcgccaaacaccgtcgccttggactggaagctgcacgggctgaagtcggggtgctcggccagcgtcgccgcctgccggggaggctggcccagggtccccggcgcatagcggccaacgctcagctcatccgcggcgtcggcgcccagcaggaacgagtccacgtagtagttgcccagggccccagtggtggccatcaccgtgcccagcgcctggcccgcccggcccgacccacggaaattatgaaactgcagatttcatgtaacaacttggtggcaccgggggggaagtacagtcacctaataagttgccggcgcccgcgcccccattggccgtgcgcgtcacgtgcccgtccagcagaacaataacgcgtaaatcactccgcacgctattaatggtccgatgttttgcagtcataatttttatagcaaaagccatatgtttttatgtaaagggatcgtgccgctctacgatggggtttgttttaattgtggccaacgacgattaaaagatcaaatctagccttgtctctgtactctcccgtctccccccccatacacacacttcttaagcggactattttatatcacaattaatcacgccatcaagaaggcgcgggtcccgcgtgcgagtgcggccagcggagcccctcacataaaattagacaataattgaagccataaaaaagcagccaaatcgcattgtcgctctactgtatttaaatctatatttatgatatttcataaggagttattgtttcagaagccacacaggctggcgggaagtcggaaacgaccaacagattcgtttgcctcgccgtggctcccagctgtaaaaatttacgaggacttggaaaggttagactgttgtgtttggttggcgagctccctgtaaataatccctgcggtccccgggagaggcgagtttacccgcggccgccctcgaaaagtcaaattcaacgcaggatccgtcccaaacggagccgccgccggccctaccagggcactccaggcagggaccggccgctcagggagtaccgcgggtgtaggtccccacagctacccgcctggagcgaggggcgcccgggcaacccttaaattcgcctttgctacgaggaccccacggaggagctggccaggagggagcggccagccgccaccagggcgaaggttttgagggcctggttggttgtgcggcgcgctcggtccccggccctcgaccccacgcacacgcgcgcccagcccgcctttctcatcagctggcaatcaggattcccaggcgcaggcggctggcgacccagccctgtgctccagcctcagaggctctaaccatgagcgctgcaagcctggttgcgctccgtgaatcccagctggggaaaaaactacaagtggcatgaatggaaggcaagttcggtttgggaaaaggcagcctcgcctaagagaccccgcagctccggaacctgggaggcccgcaccgatgtggcctgtcccggggccgcgtgagcctttcagggctccttcctccctttccagctgctactccgggcctcgccttggttacctacggggcccggagactcggcg

SEQ ID NO: 101 - DNA, 21 bp, Artificial Sequence
  HOXC4F primer
accaggagctgtacccaccac

SEQ ID NO: 102 - DNA, 21 bp, Artificial Sequence
  HOXC4R primer
cgcagagcgactgtgatttct

SEQ ID NO: 103 - DNA, 422 bp, Artificial Sequence
  HOXC4 - CpG island Position chr1254411710- 54412131; Band 12q13.13; Genomic Size 422
cgcgactgctagagctcacacatgcgcagtgtgggcccagggccgggccgccgagcaggaagccggcgcagctaggcggccggcggggcctgttaattggcaattaggggggaggctggtggctggtgcgcgtcagccgagaggagagcgtctgcccaccccctgctcccgcccccactcgggcggatggaagggtgggaggtgccctgcgttgggtggagggtggaggttgtagggtgggggtgggggatgctgtactcaaaagccatcttgtgctcagagaaaagaggcctaccggctttcccttccggggtccggcgcccctcacccccagccgcggccatcccagccgggatgcccactggaccgggatgcccgctcgccacgcatggctgctctgggctaggacctgcctcgcctcg

SEQ ID NO: 104 - DNA, 20 bp, Artificial Sequence
  PCDHA13-G1F primer
catggtgtcgctcttcactg

SEQ ID NO: 105 - DNA, 20 bp, Artificial Sequence
  PCDHA13-G1R primer
aagccagagcagtagttgcc

SEQ ID NO: 106 - DNA, 1069 bp, Artificial Sequence
  PCDHA13 - CpG island  Position chr5140263086 -140264154; Band 5q31.3; Genomic Size 1069
cgccctggaccgcgagagcgtatcagcctatgaactggtggtgaccgcgcgggacgggggctcgccttcgctgtgggccacggccagcgtgtcggtgggggtggccgacgtgaacgacaacgcgccggcgttcgcgcagcccgagtacacggtgttcgtgaaggaaaacaatccgccgggctgccacatcttcacggtgtctgctcaggacgcggacgcacaggagaacgcgctggtctcctactcgctggtggagcggcgggtgggcgagcgtgcgctgtcgagctacgtgtcggtgcacgcggagagcggcaaggtgtacgcgctgcagccgttggaccacgaggagctggagctgttgcagttccaggtgagcgcgcgcgactctggcgtgccgcctctgggcagcaacgtgacgctgcaggtgttcgtgctggacgagaacgacaacgctccggcgctgctgacgcccggggctggcagcgcgggaggcacagtgagcgagctgatgccgcggtcggtgggtgcaggccacgtggtggcgaaggtgcgcgcggtggacgccgattcgggctacaatgcgtggctttcgtatgaattgcagctggcggcggtcggcgcgcgcatcccgttccgcgtggggctgtacactggcgagatcagcacgacgcgccctctggacgaggtggacgcgccgcaccaccgccttctggtgctggtgaaggaccacggtgagcccgcgctgacggccacggcaacggtgctgttgtcgctggtggagagcggccaagcgccacaggcttcgtcgagggcgtcggcaggcgctgtgggtccagaagcggcgctggtggatgtcaatgtttacttgatcattgccatctgcgcggtgtccagcctgttggtgctcacgttgctgctgtatactgcgctgcggtgctcggcaccgcccaccgagggcgcgtgcgcgccgggcaagcccactctagtgtgctccagcgcggcagggagttggtcgtactcgcagcagaggcggccgagggtgtgctctggggagggcccgcataagacg

SEQ ID NO: 107 - DNA, 20 bp, Artificial Sequence
  HIC1-GF primer
ctcccctcctccgtatcact

SEQ ID NO: 108 - DNA, 20 bp, Artificial Sequence
  HIC1-GR primer
gggcttccgagaagaaaact

SEQ ID NO: 109 - DNA, 4340 bp, Artificial Sequence
  HIC1 - CpG island Position chr171952920- 1962328; Band 17p13.3; Genomic Size 9409
cctccggccggctcagtcccctccccactccccaactctgcccgacgctccgaccccagcggggagattcacagtgagaatgggtgtggtcgcaagggccggaggtagggctaggagtgccccgacagtgacacccctccccctctaagagcagcgcggagccgggggagggggccgacgaaccacaggaagaggcgggaggggcctggggtctcctttggtcaaagctgatatcaaaaatataaatttcccttaccccatcccacccccgtcccggggttctcccccgacccccgagctaaggcacgaagcagtgaggccaggtgaggccgccgagaggtggagccgccactgtggcgacgctgcggttgtcccgggcacagtgggccctgcgcgccgcccccgccgctccctggggtgcgggccagggccgcgcagcagcgacagagcgggctggcgaggggcgctctaggtgggagagaaacggtcgatggtccggccgtcgggcccggccgccaggtgagcgccctggctcagcacctcggccgccttgtcggggctgaggcccagctcggccgtgaacttggccagcgggtagaggctctccagcgccaccttggggtcgtgcaggaagtgcgtggtctgcgccagcagctcggccgcggccgccttgtcctgctgcttcaggctcagctgctcggccgtgaggcgagccacagcaaagacgccctcggggaagtcgagcttgcccttgccgtcggggccggggacgccggggagcccccccaagcccgccagcgccccggccgcgccggccgcgccccccacggcgtgcatcttcatgtggctgatgaggttgcgttgctgtgcgaacttgccgccgcacacctggcactcgtagggcttctcgcccgagtggatgcgcatgtgctccgtgaggcggtactggcgcgtgaaccgcatgccgcacgcgtcgcacgcgaagggcttgaggcccaggtggctgcgcatgtggcgcgtcatggtcccacgctgcgtgaacttcttcccgcagatggtgcatgggtagggccgggtcagccagtgcgtcttctcgtgctgccgcagcgtggccgggtccttgtagctcttgtcgcacgacgcgcagcggtagggccgcagcagctctcccaggccacccggagccccggcgaccttgtccccgccgcctccaaaagggggccctaggccggcggccccagcggccacttcggccgcctcggccctgccgtacagcgcttcctcctcctccacgtgagcctccacgtgcgcgttcagctgctcagagctggggaagcccttgccgcacggaatgcacacgtacaggttgtcaccgaagctctcgggctcgccataggccaggtgcgggcatgggtagccctcgaggtggccgccaggcgggctggggtcctcgctgctaccggtctcctcgctgctgctcttgtagtcgtcgccgtcgccgcccgcgccgggcccgtccaggctgccagggtagcgcggcggcggcgccaggccgagcgggggccccccgggcgagacggccgcgtccccaccacgctcttcgcagcgctcgctgggggagccgcgctcccggcccagctcgtcgccatagctacccaggcccggctcgtgcttcatccagcgatagaggagactaggcccgtcggggcggccggggggctcgggtcccgggctgccgctgccgccgcgaaatgggtcggaaggcggtgcggcctcctccagcttctggaagggcagcggcggcagcgacggcagggcgagaggcggctccttgtaggcggcggggccggcgctgggagggctgtccgggcgcgggggcagctcgcgctcagccagcggccgctctggcgccgcggagcccggcgggctcttcttggacaggtccaggccacaaagaggggagcagcggcgctccgaggcacagagtgcggcggccgggccgggtcccgacgcgtacagctcggcgcagtgcgtgttgaccgcggcctctgggcccgagggcggctccgcggcaggcggcggcggaggcccgactggggacgggtagcaggcctggatgaccggcgtggcggcccgcaggccccggcccggccgaccatagggcgcgtagccgccgccgccgccgccgccgccccgcaggtggcagtacttgccgtggcgcttgaggcgtttcttgcacagcgccacgaggtcggggatctgcaggtagctggcggcggccagcacggcgcccaggctcggctcagcccccggggccacggccgcggccgcagccgcctctgcgccgtcagccaggcggccggtgtagatgaagtccagcaccaggcggaacacggccgggctcaccatgtcatggtccaggttgagcaggttgtcatgcaccaccagggacttgaggtaggcgctgctggccgccagcacgttcttgtgcgcgcggaagagggcgttctgcaccacgatgatcacgtcgcacaagaagcccttggtgcgctggttgttgagctgcagcagcagctgcctggagtggccgggcgcctccatcgtgtccagcatcgtctgcccagcacactctcctgcggggacacacaccggccgggtgagagccgtgcggcgccctggccgcctggccccagcccggcacttctcccctccacttccccttccctcagctgagcgggggcatcagccctgcggcctgggcaccggcgaaggaccggctgccctctggagtgggagcccaggccggcccgcccggaccaggagaaggagcaggaggtgagcggccgccggtggaggggaggccagggcggcctgcacgccccagggcacctggctgggtgctggggcttccgagaagaaaactgttcaggcgcagtgacccttttggagacagttacccgatttaagtaaaatgtccgcttcaggaaaagtcattcagggcggagaactttacccaagtagggagaaagggagccgaggaaccagcgcctcccgcctcgggagaagttgccccagttgggggaagtgatacggaggaggggagcgcggtgcccgccctggcgccgccctggccgggggctgtcaaccctcggtcggggcccgggcggcggccgcgcggggagcggaggcagcggctgccgtggcgggcagagcgcgaaggccgggcccggcgcggggagggcgttatatcggggcaggaggctgaggcaggaagcaggtgggggggaggggggagccacgcagctcccaggggagggagggggcagcgccccgggcgggcacggcgcacagccggctgcggccctgacccgggcctgcgccccacccgcgtcccggcctcggcctgggccctacacgcgcgggcccggcgcctccctccgcggctcccccggccccttctcccccggaactccgccgccccaaacttggggaaaagttttccaactgcagacagggcgggaggagtgcgccggccccaggccctcggctcgcagctcttcctcgcggcccccaaatccggcggcagagcccggagccgagccctgagctcccctgcccgctgctcgcccgcccgaccccgttcccctcctggcccgcggggccccgcggcccgttacctgcggtcccggcgggccgggctcccctccccgcggcggtggcagctcttagccgatgccccacccgccgctgccaggccccgagctgtgccagggcagcgcccctgccagccccgcccgccagctccccttcccttcccttcccctcgcctctccagcccatgtgcgggcagagccggccccgggccgctgaccccgccgtgaacccggcgcggagccgcggcccggtggtcctgagtccgaaagggacgacacccggagccctgaacgccagccgccagccgcgatggggcacccgcgccagaagatgcacccgaggcggccgacgcacgaggaccgggctgtcccgggtcccccgtccctcccggtccccggctcgaggacccacctggggggcatgtcgaaagccccgggcccggctgacggcggatccaggggggacgtggctgcgctgccctccgcccgccgggcccccggtcggtctgtcctgctggtccgtcctccccgcgtcctggtcgcgtctcagccccgccgcgctttccgcacactcttatctggagcggcccgggccggcgggcgctgctgcggctatggcgccacctcgcgggcgcgcagggctctgcgcggcaggccgctgccttcctcccgcgcacctgagctgga

SEQ ID NO: 110 - DNA, 20 bp, Artificial Sequence
  CDH13-GF primer
gggagcgttaggaaggaatc

SEQ ID NO: 111 - DNA, 20 bp, Artificial Sequence
  CDH13-GR primer
aggagaacgcacagaacgag

SEQ ID NO: 112 - DNA, 1162 bp, Artificial Sequence
  CDH13 - CpG island Position chr1682660652- 82661813; Band 16q23.3; Genomic Size 1162
cgcgtgcatgaatgaaaacgccgccgggcgcttctagtcggacaaaatgcagccgagaactccgctcgttctgtgcgttctcctgtcccaggtagggaagaggggctgccgggcgcgctctgcgccccgtttctgcattcggatcgcccggcacgggcagggtgagggggctttcggggggtcggggcctccggtcgcggcggcgaagacagatcggggctcggtagggaggtcattccgagcccagagatcctaggcaccccccacacacaggctcccactctggcgtgcgtgtgtgtgtgtgtgtgtgtgtgtgtgtgtgtgtgtgtgtgtgtacgttcgttaacgggaggaggagagagctcccagtccttttttgctagcaggggcgacattctcgcccacatcaagtggggtaactttggttccctcctccggaggctcggtgcattggagaaagactcagttagaggcgactccaacgagccgcggttttccccagcccaacgcccagcggccgaagcgctgctcgggtccggattgcgggatgcggggctggagaggccgagcaggcaccaccgacttcccagggcgcccgggccccctggtacagcccggctgcccgctggaaggcgcctcggggcagcagagagcctcagcccggctgctgctgtcgctcaaaggcgccggcgccggccgcacccgcatcggggtccttttgctcccagaccccgggcccgaaagggccggagcgtgtcccccgccagggcgcaggccccagccccccgcacccctattgtccagccagctggagctccggccagatcccgggctgccgcctctgctgccttccctgagcgggagcggagcgcagagaaaagttcaagccttgcccacccgggctgcagctgcttgttaaccctcagagcgccacggcgcgagggaagggcacgccaaccaggagagggggcgagggagatgcggtccgcctgcagtcacctctgcacctcagagatttcgggaagtttgagtgcaggaaagcagcgctccgaggccaggcctggggtgctggccgctgcggggggcacgccctgcgctgctcaggggcctgtggtttcggagagcaccccgatccagtcccccatcgcctctctggcaggcg

SEQ ID NO: 113 - DNA, 22 bp, Artificial Sequence
  HOXA4F primer
tagtaggaggcagtgggctctc

SEQ ID NO: 114 - DNA, 22 bp, Artificial Sequence
  HOXA4R primer
aaaacgacaacgcgagaaaaat

SEQ ID NO: 115 - DNA, 1066 bp, Artificial Sequence
  HOXA4 - CpG island Position chr727169573- 27170638; Band 7p15.2; Genomic Size 1066
cggctggctggcgcgcacatacccacatctcaccgcagcccgggtcagatgggggctcccctcccgaggcccccttcccctgagcctctccctcctgaccccgaccctcgaacccaggcccagccccggcccacctcccgcgcctcccaagcggcgccacgtaccggcgctgacatggatcttcttcatccaggggtacaccacgggctccttgcccttcaggcccagcgggctcttgtcggccaagagcagcgggcacgcgggggcgctgccccctgccgggacgcctggggtggcgggggccgcctcgcagcgccgcggggccgctgggggcacggcgcgaggctgcaggggcggcggcagctggggctgcaggacgtggctcgcatgcaggccgtgcgctgggcccttggcttgcgccgggggctgctcgggctggggcggccgcccggggctggcgccgccgcggtagccataggggtaggcggtgtccgcggccccatgcgcggggtacagcgcggcagcagggtaggcgggctcgcgggcggtccgcggcgcgtagtaggaggcagtgggctctcggccgccgcccgcgtgagggagctggggctgctgcagcggcaggtgctgggtcgggggcgctgggggctgctggtagccggggcccccgcccgggccgccgtctgcgccgcccgagccgctgtgctgcgcgtactcctcgaagggagggaacttgggctcgatgtagttggagtttatcaaaaacgagctcatggtcattaatttgtgaagtgcaaaaatactaatttttctcgcgttgtcgttttttctgggcttgccgaggcccctccccctcctgcctcgcttcccatccccctttcctctgcgcccttcccctccccccgctgtcaagtgcccactcctccccctcccgcagacgccgccaccaaagttcgagccgctcctccccagcccagcgcgcgccccgccccgtgccccacgtgcagcgcccccaccaatgggcgcaccgcgcgcgcggacccggatcaggaaacgcgcgggtgcg

SEQ ID NO: 116 - DNA, 20 bp, Artificial Sequence
  PCDHA6-G1F primer
ctgactgttgaatgatggcg

SEQ ID NO: 117 - DNA, 20 bp, Artificial Sequence
  PCDHA6-G1R primer
tcgggtacggagtagtggag

SEQ ID NO: 118 - DNA, 353 bp, Artificial Sequence
  PCDHA6 - CpG island Position chr5140207726- 140208078; Band 5q31.3; Genomic Size 353
cgcttctgctcctcgcagcctggaaggtggggagcggccagctccactactccgtacccgaggaggccaaacacggcaccttcgtgggccggatcgcgcaggacctggggctggagctggcggagctggtgccgcgcctgttcaggatggcctccaaagaccgcgaggaccttctggaggtaaatctgcagaatggcattttgtttgtgaattctcggatcgaccgcgaggagctgtgcgggcggagcgcggagtgcagcatccacctggaggtgatcgtggacaggccgctgcaggttttccatgtggacgtggaggtgagggacattaacgacaacccgcccttgttcccg

SEQ ID NO: 119 - DNA, 20 bp, Artificial Sequence
  PCDHB15-G1F primer
aagcctgttagcagagcacg

SEQ ID NO: 120 - DNA, 20 bp, Artificial Sequence
  PCDHB15-G1R primer
tccatcacagaatagcgacg

SEQ ID NO: 121 - DNA, 929 bp, Artificial Sequence
  PCDHB15 - CpG island Position chr5140626445- 140627373; Band 5q31.3; Genomic Size 929
cgagcagagcataaccgtgctggtgtcggacgtcaatgacaacgcccccgccttcacccaaacctcctacaccctgttcgtccgcgagaacaacagccccgccctgcacatcggcagtgtcagcgccacagacagagactcgggcaccaacgcccaggtcacctactcgctgctgccgccccgggacccgcacctgcccctcacctccctggtctccattaacacggacaacggccacctgttcgctctccagtcgctggactacgaggccctgcaggctttcgagttccgcgtgggcgccacagaccgcggcttcccggcgctgagcagcgaggcgctggtgcgagtgctggtgctggacgccaacgacaactcgcccttcgtgctgtacccgctgcagaacggctccgcgccctgcaccgagctggtgccccgggcggccgagccgggctacctggtgaccaaggtggtggcggtggacggcgactcgggccagaacgcctggctgtcgtaccagctgctcaaggccacggagcccgggctgttcggcgtgtgggcgcacaatggcgaggtgcgcaccgccaggctgctgagcgagcgcgacgtggccaagcacaggctagtggtgctggtcaaggacaatggcgagcctccgcgctcggccaccgccacgctgcaagtgctcctggtggacggcttctctcagccctacctgccgctcccagaggcggccccggcccaagcccaggccgactcgcttaccgtctacctggtggtggcattggcctcggtgtcttcgctcttcctcttctcggtgttcctgttcgtggcagtgcggctgtgcaggaggagcagggcggcctcagtgggtcgctgctcggtgcccgagggcccctttccagggcatctggtggacgtgagcggcaccg

SEQ ID NO: 122 - DNA, 22 bp, Artificial Sequence
  PTPN6-GF primer
ttcgcatgcgtgaagtattatc

SEQ ID NO: 123 - DNA, 20 bp, Artificial Sequence
  PTPN6-GR primer
agctcagggactaagcctca

SEQ ID NO: 124 - DNA, 629 bp, Artificial Sequence
  PTPN6 - CpG island Position chr127079501- 7080129; Band 12p13.31; Genomic Size 629
cgtggaggggcgcggggacagggcaaggggtttgggggagggactggaagcgtccggcgagcaggcggaggttgctcaccggtgaacacagattcgcgcacaccgtaggccacggcgccggcccccagcaacagcttcagggccgtgcccatgccccggggcccggcgggcagccgtcccgccaagtccttcaagttctgggccatgtctgatcttgaggccggcggcactggaggtcagaagggggtgccggcccgcctctaccccgctccggcttaggtactgcacccttcacacgagggttcgggcccgtaaggctggcgaaagaaagggcagcggaagtgcgctccctttgaaaccctcccccttagcccactacggacccgaacttcgcgcacaggaatcgcgcatacggaagtcccgcccctttctggaaggctgccctcccagggagggcagcgcaagacagcaagtcatctccatttcctggcccactttcaaaatggcagccggaaggaaatttgtgattagaagccgcgctgttcttatttaagagcgttagcgcaacttccggtattgttgcaagatggccgcgcccagtgatggattcaagcctcgtgaacgaagcg

SEQ ID NO: 125 - DNA, 20 bp, Artificial Sequence
  APC-GF primer
gaagcagctgtgtaatccgc

SEQ ID NO: 126 - DNA, 20 bp, Artificial Sequence
  APC-GR primer
aagacagtgcgagggaaaac

SEQ ID NO: 127 - DNA, 838 bp, Artificial Sequence
  APC - CpG island Position chr5112043080- 112043917; Band 5q22.2; Genomic Size 838
cgggacagaacagcgaagcagtgcccggcaagcggagcgcagcacccattgcgcctgcgcataacaggctctagtctccgggctgtgggaagccagcaacacctctcacgcatgcgcattgtagtcttcccacctcccacaagatggcggagggcaagtagcaagggggcggggtgtggccgccggaagcctagccgctgctcgggggggacctgcgggctcaggcccgggagctgcggaccgaggttggctcgatgctgttcccaggtactgttgttggctgttggtgaggaaggtgaagcactcagttgccttctcgggcctcggcgccccctatgtacgcctccctgggctcgggtccggtcgcccctttgcccgcttctgtaccaccctcagttctcgggtcctggagcaccggcggcagcaggagctgcgtccggcaggagacgaagagcccgggcggcgctcgtacttctggccactgggcgagcgtctggcaggtgagtgaggctgcaggcattgacgtctcctcccggcaaagcttcctcggctttgccccgccgctgctcgggaccctacggtgctcggcccgactctgtggctctcttctctccatgtctcaccctctcccctccccgcactccccattcaggcctccagttggcccctggctttgcaggtcctccattctcacgcagtggatgggggtcgcgacgcccgccgtcctccacctttcctggctgctgctggagcttcgcccctgcaagtggtgccccattcgcgttaggtgggtgggtcgtccgcccttcccattttagtcgcttccccatcttcctcg

SEQ ID NO: 128 - DNA, 20 bp, Artificial Sequence
  GSTP1-GF primer
tttcctttcctctaagcggc

SEQ ID NO: 129 - DNA, 20 bp, Artificial Sequence
  GSTP1-GR primer
ctttccctctttcccaggtc

SEQ ID NO: 130 - DNA, 1025 bp, Artificial Sequence
  GSTP1 - CpG island Position chr1167350929- 67351953; Band 11q13.2; Genomic Size 1025
cgggtgtgcaagctccgggatcgcagcggtcttagggaatttccccccgcgatgtcccggcgcgccagttcgctgcgcacacttcgctgcggtcctcttcctgctgtctgtttactccctaggccccgctggggacctgggaaagagggaaaggcttccccggccagctgcgcggcgactccggggactccagggcgcccctctgcggccgacgcccggggtgcagcggccgccggggctggggccggcgggagtccgcgggaccctccagaagagcggccggcgccgtgactcagcactggggcggagcggggcgggaccacccttataaggctcggaggccgcgaggccttcgctggagtttcgccgccgcagtcttcgccaccagtgagtacgcgcggcccgcgtccccggggatggggctcagagctcccagcatggggccaacccgcagcatcaggcccgggctcccggcagggctcctcgcccacctcgagacccgggacgggggcctaggggacccaggacgtccccagtgccgttagcggctttcagggggcccggagcgcctcggggagggatgggaccccgggggcggggagggggggcagactgcgctcaccgcgccttggcatcctcccccgggctccagcaaacttttctttgttcgctgcagtgccgccctacaccgtggtctatttcccagttcgaggtaggagcatgtgtctggcagggaagggaggcaggggctggggctgcagcccacagcccctcgcccacccggagagatccgaacccccttatccctccgtcgtgtggcttttaccccgggcctccttcctgttccccgcctctcccgccatgcctgctccccgccccagtgttgtgtgaaatcttcggaggaacctgtttccctgttccctccctgcactcctgacccctccccgggttgctgcgaggcggagtcggcccggtccccacatctcgtacttctccctccccgcaggccgctgcgcggccctgcg

SEQ ID NO: 131 - DNA, 24 bp, Artificial Sequence
  ADAM12-AF primer
cgctgagctcttctagcctttcat

SEQ ID NO: 132 - DNA, 24 bp, Artificial Sequence
  ADAM12-AR primer
tccgcggatataagaacggtgact

SEQ ID NO: 133 - DNA, 1327 bp, Artificial Sequence
  ADAM12 - CpG island Position chr10128076156- 128077482; Band 10q26.2; Genomic Size 1327
cggggccgctgcgcgcccccctaagtgtgttagcgggggaggcggggctggaaaggaaacctggtgaagggctggcccggagcctggggtggggatattcactgcgggatagggccagcaagaggacccgacacgcatcgtcccgagtgacacgtgtaaatgtcaagatacagagacatctgcaaatgtcacccaagagggtgaggacgggggagcggtcccgaggctgtgccctccggggcaggtactggctcctgtggggctgcgggccaagtgtcgcccttccccaaggaattggcacctggggggggggggtcggtctcgccgcgctggaagcgcaagccccggggctccggagatgcgccggggcgcgtcgcccctcggggcagccctggacctcggcgcgcccaggcgcagcgtgcggtgccctcggcggggcgggcagcgagccgccctagttcggcgacttacctcgggcctcgcagggcgcgagcagagcaccggccagggcgagcaggagggcgcgggcgggggacacgggcagcgggcgcgctgccatcgtcgccggccttcagtgcagcagctctcgggcccggcggcgagcgctgcaccatcccacgcgggcgccgagccggggccgggcgtcgcgaccggagggatttcctgcctcggcgagtcagctccggagccctcgcgcagcgcccgcgccgccgctgagctcttctagcctttcatttttaaaaaagtttccccccgtgtgtgtgcgtgcgtgcgcgcgcgcgcgccgttctggcacaagccagccttgaccgttgcaataaatgagcaaactgtccgagttggcccggggactaggaagagcgttagtgagagaaggcaggcctgtgaaatggatccacggccagcagtcaccgttcttattaccgcggaacaaattattgtctcccccgcacccccgccagttggcggcgtcccgcgggtcctagagaccgctcgggtccccccgccagggtcccgccccgagccgcggctcgctcacccccgagggtgggcggctcagacgtggctcagtggcgtccgggcgcccggagcgcacacgtccccgccccaggatgatgtggccgcagggcccggggcgcccggctgccaagcgcacatgcggcggcacggtccagcttttcaggctgaagctggaaacgatgactctgctactcgctccccggctctctgggaaccctcggagtgcgggtcaggtctccaccgcggcccacagcccggcgcgcgaccccgcccggccctaagcgcccaaaggggcatctctcgcccg

SEQ ID NO: 134 - DNA, 20 bp, Artificial Sequence
  p16-GF primer
ctcctctttcttcctccggt

SEQ ID NO: 135 - DNA, 17 bp, Artificial Sequence
  p16-GR primer
ccttccttgccaacgct

SEQ ID NO: 136 - DNA, 370 bp, Artificial Sequence
  p16 - CpG island  Position chr921968359- 21968728; Band 9p21.3; Genomic Size 370
cgcaatggcttcacgtgcatgtacccgccgccaccgctctcccacacctccctggtccagcagctagtccactgcccgcctggctgctccaggcgcgccgaccgctcaagcgctccaggtccacccggcggagggcagagaaagcgcgaccgcgcggcccgcagggttgcaagaagaaaacgagtgttatataatgagtctcagtggttgctcacaatgccaggcgcgaaggcgtgaagatgtggcctttcccttcccgcatccccaggcatcttttgcacctggtgcggagtgagccagccagcttgcgataaccaaagggcgcctcaggctctggcgctcctcggcggaatcccgtagcttccctacg

SEQ ID NO: 137 - DNA, 20 bp, Artificial Sequence
  GABRBA-GF primer
ggacctccctgactgtcaac

SEQ ID NO: 138 - DNA, 20 bp, Artificial Sequence
  GABRBA-GR primer
cctccgggtagtcagagaca

SEQ ID NO: 139 - DNA, 728 bp, Artificial Sequence
  GABRBA - CpG island Position chr921974579- 21975306; Band 9p21.3; Genomic Size 728
cggagaatcgaagcgctacctgattccaattcccctgcaaacttcgtcctccagagtcgcccgccatcccctgctcccgctgcagaccctctacccacctggatcggcctccgaccgtaactattcggtgcgttgggcagcgcccccgcctccagcagcgcccgcacctcctctacccgaccccgggccgcggccgtggccagccagtcagccgaaggctccatgctgctccccgccgccggctccatgctgctccccgccgcccgctgcctgctctccccctctccgcagccgccgagcgcacgcggtccgccccaccctctggtgaccagccagcccctcctctttcttcctccggtgctggcggaagagccccctccgaccctgtccctcaaatcctctggagggaccgcggtatctttccaggcaaggggacgccgtgagcgagtgctcggaggaggtgctattaactccgagcacttagcgaatgtggcacccctgaagtcgccccaggttgggtctcccccgggggcaccagccggaagcagccctcgccagagccagcgttggcaaggaaggaggactgggctcctccccacctgccccccacaccgccctccggcctccctgctcccagccgcgctcccccgcctgccagcaaaggcgtgtttgagtgcgttcactctgttaaaaagaaatccgcccccgccccgtttccttcctccgcg